(19) United States
(12) Patent Application Publication
(10) Pub. No.: US 2023/0364118 A1
(43) Pub. Date: Nov. 16, 2023

(54) RIBITOL TREATMENT

(71) Applicant: THE CHARLOTTE MECKLENBURG HOSPITAL AUTHORITY D/B/A ATRIUM HEALTH, Charlotte, NC (US)

(72) Inventors: Qi Long LU, Charlotte, NC (US); Bo WU, Matthews, NC (US)

(21) Appl. No.: 18/044,747

(22) PCT Filed: Sep. 9, 2021
(86) PCT No.: PCT/US2021/049677

§ 371 Date: Mar. 9, 2023

Related U.S. Application Data
(60) Provisional application No. 63/076,761, filed on Sep. 10, 2020.

(51) Int. Cl. A61K 31/7004; A61P 21/00

(52) U.S. Cl. CPC A61K 31/7004; A61P 21/00

(57) ABSTRACT

Provided are methods of treating a disease or disorder in a subject in need thereof and associated compositions. An effective amount of ribitol is administered, thereby restoring and/or enhancing functional glycosylation of α-DG and/or treating the disease or disorder.

### CROSS-REFERENCE TO RELATED APPLICATIONS

This application claims the benefit of U.S. Provisional Application No. 63/076,761, filed Sep. 10, 2020, the content of which is hereby incorporated by reference in its entirety.

### BACKGROUND

Limb-Girdle Muscular Dystrophy Type 2i (LGMD2i), also known as LGMD R9 (Straub et al. Neuromuscular Disorders 28 (2018) 702-710), is a dystroglycanopathy caused by partial loss of function mutations in the FKRP gene. It is the most common form of dystroglycanopathy and presents predominantly as myopathic phenotypes with or without mild involvement of the central nervous system (CNS). The age of disease onset, mainly as muscle weakness, is most commonly between 10 and 20 years of age. However, first disease symptoms can also occur in subjects younger than 10 and older than 40 years old. Initial symptoms are mostly limb muscle weakness with mild calf and thigh hypertrophy. As the disease progresses, patients present with weakness in hip flexion and adduction, knee flexion and ankle dorsiflexion. Symptoms worsen with progressive muscle degeneration, fiber wasting, infiltration and accumulation of fibrosis and fat in muscle tissues. Patients eventually lose ambulation. Muscle weakness also involves the diaphragm with varying severity, leading to respiratory failure in a proportion of patients. Cardiac muscle is affected with the most severe and frequent presentation being dilated cardiomyopathy. There is no current disease modifying or curative therapy. In addition to LGMD2i, other muscular dystrophies associated with aberrant glycosylation of alpha dystroglycan (αDG) include, for example, Limb Girdle Muscular Dystrophy Type 2M, Limb Girdle Muscular Dystrophy Type 2U, Fukuyama Congenital Muscular Dystrophy (FCMD), Muscle-Eye-Brain (MEB) disease, and Walker-Warburg syndrome (WWS) (Montenaro and Carbonetto, Neuron, 2003, Vol. 37, 193-196).

In healthy muscle cells, the sugar chain on the protein of alpha dystroglycan (αDG) contains tandem structures of ribitol-phosphate, a pentose alcohol that was previously unknown in humans. The genes fukutin (FKTN), fukutin-related protein (FKRP), and isoprenoid synthase domain-containing protein (ISPD) encode essential enzymes for the synthesis of this structure. ISPD metabolically converts ribitol-5-phosphate into CDP-ribitol, a substrate for fukutin and FKRP. Subsequently, fukutin transfers the first ribitol-phosphate onto sugar chains of αDG followed by FKRP which transfers the subsequent ribitol-phosphate.

US 2018/0169036 A1 discloses methods of treating a disorder associated with a mutation in a fukutin related protein (FKRP) gene by administering ribitol in drinking water. A need for continuous, or at least daily, administration of ribitol to achieve therapeutic effect is consistent with the expected short half-life of a pentose alcohol like ribitol. For example, the closely related pentose sugar D-ribose has a short half-life, 14-24 minutes in rabbits (Alzoubi et al, 2018).

Thus, there remains an unmet need for methods of treatment using ribitol in humans.

### SUMMARY

The present disclosure provides compositions and methods for treating diseases or disorders. For example, the present disclosure provides compositions comprising ribitol as well as methods of using ribitol to treat various diseases and disorders in a subject (e.g., a mammal, such as a human). Diseases and disorders for treatment according to the methods provided herein include diseases and disorders associated with a defect in Fukutin-related protein (FKRP) including muscular dystrophies such as FKRP-related alpha-dystroglycanopathy or Limb-Girdle Muscular Dystrophy type 2i (LGMD2i).

In an aspect, the present disclosure relates to a method of treating a disease or disorder in a subject in need thereof, comprising administering an effective amount of ribitol, thereby restoring and/or enhancing functional glycosylation of αDG and/or treating the disease or disorder.

In some embodiments, the dose is administered at most four times daily. In some embodiments, the dose is administered at most twice daily. In some embodiments, the dose is administered three times daily. In some embodiments, the dose is administered twice daily. In some embodiments, the dose is administered once daily.

In some embodiments, the method comprises administering at least about 0.5 grams (g)/day, at least about 1 g/day, at least about 2 g/day, at least about 3 g/day, at least about 4 g/day, at least about 5 g/day, at least about 7.5 g/day, at least about 10 g/day, at least about 12.5 g/day, at least about 15 g/day, at least about 20 g/day, at least about 25 g/day, at least about 30 g/day, at least about 35 g/day, at least about 40 g/day, at least about 45 g/day, at least about 50 g/day, at least about 55 g/day, at least about 60 g/day, at least about 70 g/day, at least about 80 g/day, at least about 90 g/day, at least about 100 g/day, at least about 110 g/day, at least about 120 g/day, at least about 130 g/day, at least about 140 g/day, at least about 150 g/day, at least about 160 g/day, at least about 170 g/day, at least about 180 g/day, at least about 190 g/day, at least about 200 g/day, or at least about 210 g/day.

In some embodiments, the method comprises administering at most about 0.5 g/day, at most about 1 g/day, at most about 2 g/day, at most about 3 g/day, at most about 4 g/day, at most about 5 g/day, at most about 7.5 g/day, at most about 10 g/day, at most about 12.5 g/day, at most about 15 g/day, at most about 20 g/day, at most about 25 g/day, at most about 30 g/day, at most about 35 g/day, at most about 40 g/day, at most about 45 g/day, at most about 50 g/day, at most about 55 g/day, at most about 60 g/day, at most about 70 g/day, at most about 80 g/day, at most about 90 g/day, at most about 100 g/day, at most about 110 g/day, at most about 120 g/day, at most about 130 g/day, at most about 140 g/day, at most about 150 g/day, at most about 160 g/day, at most about 170 g/day, at most about 180 g/day, at most about 190 g/day, at most about 200 g/day, or at most about 210 g/day.

In some embodiments, the method comprises administering about 0.5 g/day, about 1 g/day, about 1.5 g/day, about 2 g/day, about 3 g/day, about 4 g/day, about 5 g/day, about 6 g/day, about 7.5 g/day, about 10 g/day, about 12 g/day, about 12.5 g/day, about 15 g/day, about 20 g/day, about 25 g/day, about 30 g/day, about 32 g/day, about 35 g/day, about 40 g/day, about 45 g/day, about 50 g/day, about 55 g/day, about 60 g/day, about 70 g/day, about 80 g/day, about 90 g/day, about 100 g/day, about 110 g/day, about 120 g/day, about 130 g/day, about 140 g/day, about 150 g/day, about 160 g/day, about 170 g/day, about 180 g/day, about 190 g/day, about 200 g/day, or about 210 g/day.

In some embodiments, the method comprises administering about 0.5 g/day. In some embodiments, the method comprises administering about 1.5 g/day. In some embodiments, the method comprises administering about 3 g/day. In some embodiments, the method comprises administering about 6 g/day. In some embodiments, the method comprises administering about 10 g/day. In some embodiments, the method comprises administering about 12 g/day. In some embodiments, the method comprises administering about 15 g/day. In some embodiments, the method comprises administering 24 g/day. In some embodiments, the method comprises administering 25 g/day. In some embodiments, the method comprises administering 30 g/day. In some embodiments, the method comprises administering 32 g/day. In some embodiments, the method comprises administering 35 g/day. In some embodiments, the method comprises administering 40 g/day. In some embodiments, the method comprises administering 45 g/day. In some embodiments, the method comprises administering 50 g/day. In some embodiments, the method comprises administering 55 g/day. In some embodiments, the method comprises administering 60 g/day.

In some embodiments, the method comprises administering the dose of ribitol for at least one week, two weeks, four weeks, or longer. In some embodiments, the method comprises administering the dose of ribitol for at least one month, two months, four months, six months, eight months, ten months, 12 months, 14 months, 16 months, 18 months, or longer. In some embodiments, the method comprises administering the dose of ribitol chronically, such as for at least six months or longer.

In some embodiments, the disease or disorder is associated with a defect in Fukutin-related protein (FKRP). In some embodiments, a mammal has a mutation in the gene encoding Fukutin-related protein (FKRP) that causes a partial or complete loss-of-function in FKRP. In some embodiments, the disease or disorder is a muscular dystrophy. In some embodiments, the muscular dystrophy is FKRP-related alpha-dystroglycanopathy.

In some embodiments, the disease or disorder is Limb-Girdle Muscular Dystrophy type 2i (LGMD2i). In some embodiments, the muscular dystrophy is a fukutin (FKTN)-related alpha-dystroglycanopathy. In some embodiments, the muscular dystrophy is Limb-Girdle Muscular Dystrophy Type 2M (LGMD2M). In some embodiments, the muscular dystrophy is Limb-Girdle Muscular Dystrophy Type 2U (LGMD2U).

In some embodiments, the FKTN-related alpha-dystroglycanopathy is Fukuyama Syndrome. In some embodiments, the disease or disorder is an Isoprenoid Synthase Domain-Containing Protein (ISPD)-related alpha-dystroglycanopathy. In some embodiments, the disease or disorder is Muscle Eye Brain Disease (MEB). In some embodiments, the disease or disorder is Congenital Muscular Dystrophy (CMD).

In some embodiments, the maximum observed concentration (Cₘₐₓ) of ribitol is between about 50 micrograms per milliliter (μg/mL) and about 2500 μg/mL.

In some embodiments, the area under the plasma concentration-time curve (AUC₀₋₂₄) for ribitol is between about 100 microgram·hour per milliliter [(μg·h)/mL] and about 8000 (μg·h)/mL or between about 350 microgram·hour per milliliter [(μg·h)/mL] and about 8000 (μg·h)/mL.

In some embodiments, the area under the plasma concentration-time curve (AUC₀₋₂₄) for ribitol is at least about 100 microgram·hour per milliliter [(μg·h)/mL] or about 100 microgram·hour per milliliter [(μg·h)/mL] to about 700 (μg·h)/mL. In some embodiments, the area under the plasma concentration-time curve (AUC₀₋₂₄) for ribitol is at least about 182 microgram·hour per milliliter [(μg·h)/mL] or about 182 microgram·hour per milliliter [(μg·h)/mL] to about 700 (μg·h)/mL. In some embodiments, the area under the plasma concentration-time curve (AUC₀₋₂₄) for ribitol is at least about 200 microgram·hour per milliliter [(μg·h)/mL] or about 200 microgram·hour per milliliter [(μg·h)/mL] to about 700 (μg·h)/mL. In some embodiments, the area under the plasma concentration-time curve (AUC₀₋₂₄) for ribitol is at least about 700 microgram·hour per milliliter [(μg·h)/mL] or about 500 microgram·hour per milliliter [(μg·h)/mL] to about 700 (μg·h)/mL.

In some embodiments, the subject is a mammal. In some embodiments, the subject is a human. In some embodiments, the subject is a human child.

In some embodiments the method of any of the preceding embodiments treats the disease or disorder.

In another aspect, provided herein is a method of treating a disease or disorder in a subject in need thereof, comprising administering ribitol at dose effective to achieve steady-state AUC(0-24) level.

In some embodiments, the dose is administered at most four times daily. In some embodiments, the dose is administered at most twice daily. In some embodiments, the dose is administered three times daily. In some embodiments, the dose is administered twice daily. In some embodiments, the dose is administered once daily.

In some embodiments, the method comprises administering at least 0.5 grams per day (g/day), at least 1 g/day, at least 2 g/day, at least 3 g/day, at least 4 g/day, at least 5 g/day, at least 7.5 g/day, at least 10 g/day, at least 12.5 g/day, at least 15 g/day, at least 20 g/day, at least 25 g/day, at least 30 g/day, at least 35 g/day, at least 40 g/day, at least 45 g/day, at least 50 g/day, at least 55 g/day, or at least 60 g/day.

In some embodiments, the method comprises administering at most 0.5 g/day, at most 1 g/day, at most 2 g/day, at most 3 g/day, at most 4 g/day, at most 5 g/day, at most 7.5 g/day, at most 10 g/day, at most 12.5 g/day, or at most 15 g/day, at most 20 g/day, at most 25 g/day, at most 30 g/day, at most 35 g/day, at most 40 g/day, at most 45 g/day, at most 50 g/day, at most 55 g/day, or at most 60 g/day.

In some embodiments, the method comprises administering 0.5 g/day, 1 g/day, 1.5 g/day, 2 g/day, 3 g/day, 4 g/day, 5 g/day, 6 g/day, 7.5 g/day, 10 g/day, 12 g/day, 12.5 g/day, 15 g/day, 20 g/day, 25 g/day, 30 g/day, 35 g/day, 40 g/day, 45 g/day, 50 g/day, 55 g/day, or 60 g/day. In some embodiments, the method comprises administering 0.5 g/day. In some embodiments, the method comprises administering 1.5 g/day. In some embodiments, the e method comprises administering 3 g/day. In some embodiments, the method comprises administering 6 g/day. In some embodiments, the method comprises administering 10 g/day. In some embodiments, the method comprises administering 12 g/day. In some embodiments, the method comprises administering 15 g/day. In some embodiments, the method comprises administering 20 g/day. In some embodiments, the method comprises administering 25 g/day. In some embodiments, the method comprises administering 30 g/day. In some embodiments, the method comprises administering 35 g/day. In some embodiments, the method comprises administering 40 g/day. In some embodiments, the method comprises administering 45 g/day. In some embodiments, the method comprises administering 50 g/day. In some embodiments, the method comprises administering 55 g/day. In some embodiments, the method comprises administering 60 g/day.

In some embodiments, the method comprises administering the dose of ribitol for at least one week, two weeks, or four weeks. In some embodiments, the method comprises administering the dose of ribitol for at least one month, two months, or four months. In some embodiments, the method comprises administering the dose of ribitol chronically.

In some embodiments, the disease or disorder is associated with a defect in Fukutin-related protein (FKRP). In some embodiments, a mammal has a mutation in the gene encoding Fukutin-related protein (FKRP) that causes a partial or complete loss-of-function in FKRP. In some embodiments, the disease or disorder is a muscular dystrophy. In some embodiments, the muscular dystrophy is FKRP-related alphadystroglycanopathy. In some embodiments, the disease or disorder is Limb-Girdle Muscular Dystrophy type 2i (LGMD2i). In some embodiments, the muscular dystrophy is a fukutin (FKTN)-related alpha-dystroglycanopathy. In some embodiments, the FKTN-related alpha-dystroglycanopathy is Fukuyama Syndrome.

In some embodiments, the maximum observed concentration (Cₘₐₓ) of ribitol is between 50 and 2500 μg/mL.

In some embodiments, the area under the serum concentration-time curve (AUC0-24) for ribitol is between 350 (μg·h)/mL and 8000 (μg·h)/mL. In some embodiments, the AUC0-24 for ribitol is at least about 100 (μg·h)/mL. In some embodiments, the AUC0-24 for ribitol is at least about 182 (μg·h)/mL. In some embodiments, the AUC0-24 for ribitol is at least about 200 (μg·h)/mL. In some embodiments, the AUC0-24 for ribitol is at least about 700 (μg·h)/mL.

In some embodiments, the subject is a mammal. In some embodiments, the subject is a human. In some embodiments, the subject is a human child.

In some embodiments, the method restores and/or enhances functional glycosylation of α-DG. In some embodiments, the method treats the disease or disorder.

Another aspect of the present disclosure relates to a pharmaceutical composition, comprising ribitol and a pharmaceutically acceptable carrier or excipient. In some embodiments, the pharmaceutical composition is a solid, optionally a tablet, capsule, or powder. In some embodiments, the pharmaceutical composition is a solution. In some embodiments, the carrier is water, substantially pure water, or saline. In some embodiments, the pharmaceutical composition comprises ribitol at between about 0.05 grams per milliliter (g/mL) and about 10 g/mL.

In some embodiments, the present disclosure relates to a kit comprising the aforementioned pharmaceutical composition and instructions for use in treating a disease or disorder.

In another aspect, the present disclosure relates to a unit dose, comprising between about 0.5 g and about 210 g of ribitol. In some embodiments, the unit dose comprises about 12 g of ribitol. In some embodiments, the unit dose comprises about 24 g of ribitol. In some embodiments, the unit dose comprises between 0.5 g and 60 g of ribitol. In some embodiments, the unit dose comprising 0.5 g of ribitol. In some embodiments, the unit dose comprises 1.5 g of ribitol. In some embodiments, the unit dose comprises 3 g of ribitol. In some embodiments, the unit dose comprises 6 g of ribitol. In some embodiments, the unit dose comprises 9 g of ribitol. In some embodiments, the unit dose comprises 12 g of ribitol. In some embodiments, the unit dose comprises 15 g of ribitol.

In some embodiments, the ribitol in the unit dose is dissolved in water.

In another aspect, provided herein is a unit dose, comprising an amount of ribitol that is effective to achieve a steady-state AUC(0-24) level for ribitol of between 100 (μg·h)/mL and 8000 (μg·h)/mL. In some embodiments, the AUC0-24 for ribitol is at least about 100 (μg·h)/mL or about 100 (μg·h)/mL to about 700 (μg·h)/mL. In some embodiments, the AUC0-24 for ribitol is at least about 182 (μg·h)/mL or about 182 (μg·h)/mL to about 700 (μg·h)/mL. In some embodiments, the AUC0-24 for ribitol is at least about 200 (μg·h)/mL or about 200 (μg·h)/mL to about 700 (μg·h)/mL. In some embodiments, the AUC0-24 for ribitol is at least about 700 (μg·h)/mL or about 500 (μg·h)/mL to about 700 (μg·h)/mL.

In some embodiments, the unit dose is formulated as a solid, optionally a tablet or capsule. In some embodiments, the unit dose is formulated as a liquid, optionally wherein the ribitol is dissolved in water. In some embodiments, the unit dose is formulated for oral administration.

In another aspect, this disclosure relates to the pharmaceutical composition or the unit dose of any one of the preceding embodiments for use in a method of treatment according to any one of the preceding embodiments or provided herein.

### BRIEF DESCRIPTION OF FIGURES

FIG. 1 is a diagram of the model for ribitol-induced functional glycosylation of αDG in FKRP mutant cells. “*”=the first ribitol-5-phospate on the Core M3 of μ-DG is transferred by fukutin using CDP-ribitol as the donor substrate; CTP=Cytidine Triphosphate; D-Glucuronic acid (GlcA); Xylitol (Xyl); N-Acetyl-D-galactosamine (GalNAc); N-Acetyl-D-glucosamine (GlcNAc); D-Mannose (Man) (Source: Cataldi et al. 2018)

FIG. 2 is a series of immunofluorescent staining images depicting the detection of matriglycan 1 month after ribitol treatment in P448L FKRP mutant mice.

FIG. 3A is a series of immunofluorescent staining images depicting expression in matriglycan following ribitol treatment for 6 months. FIG. 3B shows the percentage of fibers that are positive for IIH6 antibody staining (aDG glycosylation present) for tibialis anterior (first bar in set of three), diaphragm (second bar in set of three), and heart (third bar in set of three) muscles across differing dose ranges. FIG. 3C is a western blot probed with IIH6 antibody showing the glycosylation of aDG over differing doses. The lower panel is a loading control probed with an actin antibody. FIG. 3D is the quantitation of FIG. 3C, the C57 wild type control mouse is used to represent 100% aDG glycosylation and the treatment group values are the percentage of wild type staining.

FIG. 4A shows a graph depicting treadmill exhaustion running distance test results for P448L FKRP mutant mice following 6 months of ribitol treatment. Statistical significance was assessed using an unpaired t test. * P≤0.05

FIG. 4B shows a graph depicting treadmill exhaustion running time test results for P448L FKRP mutant mice following 6 months of ribitol treatment. Statistical significance was assessed using an unpaired t test. * P≤0.05

FIG. 5A shows a graph depicting the whole-body plethysmography parameter Peak Inspiratory Flow in P448K FKRP mutant mice following 6 months of ribitol treatment. Statistical significance was assessed using an unpaired t test. * P≤0.05

FIG. 5B shows a graph depicting the whole-body plethysmography parameter Peak Expiratory Flow in P448K FKRP mutant mice following 6 months of ribitol treatment. Statistical significance was assessed using an unpaired t test. * P≤0.05

FIG. 5C shows a graph depicting the whole-body plethysmography parameter End-inspiratory Pause in P448K FKRP mutant mice following 6 months of ribitol treatment. Statistical significance was assessed using an unpaired t test. * P≤0.05

FIG. 5D shows a graph depicting the whole-body plethysmography parameter Tidal volume in P448K FKRP mutant mice following 6 months of ribitol treatment. Statistical significance was assessed using an unpaired t test. * P≤0.05

FIG. 5E shows a graph depicting the whole-body plethysmography parameter Expired volume in P448K FKRP mutant mice following 6 months of ribitol treatment. Statistical significance was assessed using an unpaired t test. * P≤0.05

FIG. 5F shows a graph depicting the whole-body plethysmography parameter End-Expiratory pause in P448K FKRP mutant mice following 6 months of ribitol treatment. Statistical significance was assessed using an unpaired t test. * P≤0.05

FIG. 6 is a graph depicting the serum creatine kinase levels following 6 months of ribitol treatment.

FIG. 7A is a graph depicting total body weight (g) for L276I FKRP mutant mice treated with either ribitol or saline for 1 year.

FIG. 7B is a graph depicting total distance (m) treadmill exhaustion test results for L276I FKRP mutant mice treated with either ribitol or saline for 1 year.

FIG. 7C is a graph depicting total time (s) treadmill exhaustion test results for L276I FKRP mutant mice treated with either ribitol or saline for 1 year.

FIG. 8 is a series of immunofluorescent staining images depicting matriglycan expression in L276I FKRP mutant mice following 1 year of ribitol or saline treatment.

FIG. 9 is a Western Blot depicting the protein expression of alpha-dystroglycan (αDG), beta-dystroglycan (β-DG) and GAPDH in lysates from Heart, Diaphragm (Diaph.) and Tibialis Anterior (TA) tissues from ribitol-treated (+) and untreated (−) C57/BL/6J mice following 1 month of treatment.

FIG. 10 is a graph depicting the profiles of mean plasma concentration versus time following oral administration of 0.3 or 1.0 g/kg ribitol to male and female CD-1 mice. mpk=mg/kg; h=hour

FIG. 11A is a graph depicting the mean plasma concentration versus time following 300 mg/kg oral administration of ribitol to male and female Bama Minipigs on Study Day 1.

FIG. 11B is a graph depicting the mean plasma concentration versus time following 1000 mg/kg oral administration of ribitol to male and female Bama Minipigs on Study Day 3.

FIG. 11C is a graph depicting the mean plasma concentration versus time following 300 mg/kg oral administration of ribitol to male and female Bama Minipigs on Study Day 16.

FIG. 12 is a graph depicting the profiles of mean plasma concentration versus time following IV administration of 10, 30 or 100 mg/kg ribitol to male and female CD-1 mice.

FIG. 13 is a graph depicting the profiles of mean plasma concentration versus time following IV injection of ribitol to male and female Bama Minipigs.

FIG. 14 shows creatine kinase activity measure after 1 year of oral dosing of ribitol in L276I FKRP mutant mice. C57 is the wild type mouse control. Saline represents the untreated L276I FKRP mutant mice.

FIG. 15 shows immunohistochemistry with IIH6 antibody detecting specifically the matriglycan (red membrane staining).

FIG. 16 shows levels of glycosylated αDG for all cohorts after 3-months of therapy.

FIG. 17 shows average levels in creatine kinase for cohort 1 (6 g QD) and cohort 2 (6 g BID) after 90 days of treatment.

### DETAILED DESCRIPTION

Ribitol, also known as adonitol or (2R,3s,4S)-Pentane-1,2,3,4,5-pentol, has the chemical structure below and molecular weight of 152.15 g/mol.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description is for the purpose of describing particular embodiments only and is not intended to be limiting. All publications, patent applications, patents and other references mentioned herein are incorporated by reference in their entirety. In case of a conflict in terminology, the present specification is controlling.

As used in the description of the invention and the appended claims, the singular forms “a”, “an”, and “the” are intended to include the plural forms as well, unless the context clearly indicates otherwise.

The term “about,” as used herein when referring to a measurable value such as an amount of dose (e.g., an amount of a fatty acid) and the like, is meant to encompass variations of ±20%, ±10%, ±5%, ±1%, ±0.5%, or even ±0.1% of the specified amount.

“Subject” as used herein includes is a mammal, such as primate, mouse, rat, dog, cat, cow, horse, goat, camel, sheep or a pig, preferably a human. The terms “subject” and “patient” are used interchangeably herein. In some embodiments, a patient treated in accordance with a method described herein is a human adult. In some embodiments, the patient is a human child. The age at which a patient is treated may depend on the age of diagnosis. For example, LGMD2i often presents at age 2 or 5, but may not be diagnosed until age 9. Thus, in some embodiments, a patient treated in accordance with the methods described herein is a human child of 2-5 years of age. In some embodiments, a patient treated in accordance with the methods described herein is a human child of 5-12 years of age. In some embodiments, a patient treated in accordance with the methods described herein is a human child of 12-18 years of age.

“Treat,” “treating” or “treatment” as used herein also refers to any type of action or administration that imparts a benefit to a subject that has a disease or disorder, including improvement in the condition of the patient (e.g., reduction or amelioration of one or more symptoms), healing, etc.

The term “effective amount” refers to an amount of an agent (e.g., ribitol) sufficient to have desired biochemical or physiological effect. The term “therapeutically effective amount” refers to an amount of an agent (e.g., ribitol) that is sufficient to improve the condition, disease, or disorder being treated and/or achieved the desired benefit or goal (e.g., decrease creatine kinase levels, increase in alpha-dystroglycan (αDG) levels, increase in motor control, and/or decrease in fatigue). Those skilled in the art will appreciate that the therapeutic effects need not be complete or curative, as long as some benefit is provided to the subject. The term “amount” when in reference to αDG or glycosylated αDG refers to the quantification of protein as quantified by detection of a signal obtained from the wavelength corresponding to a secondary antibody used to detect a primary antibody.

The term “enhancement,” “enhance,” “enhances,” or “enhancing” refers to an increase in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or an increase in the specified activity of at least about 5%, 10%, 25%, 35%, 40%, 50%, 60%, 75%, 80%, 90%, 95%, 97%, 98%, 99% or 100%.

The term “inhibit,” “diminish,” “reduce” or “suppress” refers to a decrease in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or a decrease or reduction in the specified activity of at least about 5%, 10%, 25%, 35%, 40%, 50%, 60%, 75%, 80%, 90%, 95%, 97%, 98%, 99% or 100%. These terms are intended to be relative to a reference or control.

The above terms are relative to a reference or control. For example, in a method of enhancing glycosylation of αDG in a subject by administering the ribitol, CDP-ribitol, ribose and/or ribulose to the subject, the enhancement is relative to the amount of glycosylation in a subject (e.g., a control subject) in the absence of administration of the ribitol, CDP-ribitol, ribose and/or ribulose.

The term “prevent,” “preventing” or “prevention of” (and grammatical variations thereof) refers to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods disclosed herein. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of administration of ribitol.

A “prevention effective” amount as used herein is an amount that is sufficient to prevent (as defined herein) the disease, disorder and/or clinical symptom in the subject. Those skilled in the art will appreciate that the level of prevention need not be complete, as long as some benefit is provided to the subject.

The active compounds described herein may be formulated for administration in a pharmaceutical carrier in accordance with known techniques. See, e.g., Remington, The Science and Practice of Pharmacy (21st Ed. 2005). In the manufacture of a pharmaceutical formulation, the active compound is typically admixed with, inter alia, an acceptable carrier. The carrier must, of course, be acceptable in the sense of being compatible with any other ingredients in the formulation and must not be deleterious to the subject. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations disclosed herein, which may be prepared by any of the well-known techniques of pharmacy comprising admixing the components, optionally including one or more accessory ingredients.

Furthermore, a “pharmaceutically acceptable” component such as a sugar, carrier, excipient or diluent of a composition according to the present disclosure is a component that (i) is compatible with the other ingredients of the composition in that it can be combined with the compositions of the present disclosure without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include any of the standard pharmaceutical carriers such as saline solutions, water, emulsions such as oil/water emulsion, microemulsions and various types of wetting agents.

As used herein, an “immediate-release dose” refers to a composition formulated for immediate bioavailability, such as a solution or suspension comprising the active ingredient (e.g., ribitol) or powder for oral administration or a tablet, capsule, or other solid formulation that does not incorporate controlled release excipients (e.g., polymer or micro-capsulation).

As used herein, a “controlled-release dose” or “extended-release dose” refers to a composition formulated for release of an active ingredient (e.g., ribitol) as a desired rate. Illustrative controlled-release doses may comprise the active ingredient formulated as a polymer based controlled release system, a micro-capsulation based controlled release system, an osmotic controlled release oral delivery system (OROS), or any combination thereof, such as a cross-linked polymer matrix loaded with an effective amount of ribitol and/or ribose. The controlled-release dose may release ribitol from and/or within polymers at a desirable rate.

Also as used herein, “and/or” refers to and encompasses any and all possible combinations of one or more of the associated listed items, as well as the lack of combinations when interpreted in the alternative (or).

Unless the context indicates otherwise, it is specifically intended that the various features described herein can be used in any combination. Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed.

It will also be understood that, as used herein, the terms example, exemplary, illustrative, and grammatical variations thereof are intended to refer to non-limiting examples and/or variant embodiments discussed herein, and are not intended to indicate preference for one or more embodiments discussed herein compared to one or more other embodiments.

All publications, patent applications, patents and other references cited herein are incorporated by reference in their entireties for the teachings relevant to the sentence and/or paragraph in which the reference is presented.

Unless the context indicates otherwise, it is specifically intended that the various features described herein can be used in any combination.

Moreover, the present disclosure also contemplates that in some embodiments, any feature or combination of features set forth herein can be excluded or omitted.

The disclosed embodiments are based on the unexpected discovery that ribitol can restore and/or enhance functional glycosylation of alpha-dystroglycan (αDG) in cells with defects in the genes related to dystroglycanopathy and cells with FKRP mutation. Thus, in one aspect, the present disclosure provides a method of restoring and/or enhancing functional glycosylation of αDG in a subject with a defect in a dystroglycan-related gene and in need thereof, comprising administering to the subject an effective amount of ribitol, thereby restoring and/or enhancing functional glycosylation of αDG in the subject.

The present disclosure also provides a method of treating a defect or abnormality in levels of the ribitol and/or CDP-ribitol in a subject, comprising administering to the subject an effective amount of a ribitol, thereby changing levels of ribitol and/or CDP-ribitol in the subject. In some embodiments, administration of an effective amount of ribitol treats a muscular dystrophy (e.g., LGMD2i) in the subject.

Furthermore, the present disclosure provides a method of treating a disorder associated with (e.g., caused by or resulting from) a mutation in a fukutin related protein (FKRP) gene in a subject, comprising administering to the subject an effective amount of a ribitol, thereby treating the disorder associated with a mutation in a fukutin related protein (FKRP) gene disorder associated with a mutation in a fukutin related protein (FKRP) gene in the subject. In some embodiments, the disorder associated with a mutation in an FKRP gene is LGMD2i.

Additionally, the present disclosure provides a method of treating muscle weakness in a subject that is a carrier of a mutated FKRP gene and/or with a mutation in a dystroglycan-related gene and/or with a defect in glycosylation of αDG, comprising administering to the subject an effective amount of ribitol, thereby treating muscle weakness. The muscle weakness can include but is not limited to weakness of skeletal muscle, cardiac muscle, and/or respiratory muscle, in any combination, in the subject.

In some embodiments, the disorder associated with muscle weakness can be associated with a defect in glycosylation of αDG, including situations without clear understanding of the underlying causes for the defect.

In some embodiments, the present disclosure provides a method of treating a muscular dystrophy disease for which restoration of and/or enhanced glycosylation of αDG would be beneficial and/or therapeutic. A nonlimiting example of a disorder associated with a mutation or loss of function in the FKRP gene is Limb-Girdle Muscular Dystrophy type 2i (LGMD2i). Certain mutations in FKRP are associated with Walker-Warburg Syndrome (WWS) and in congenital muscular dystrophy type 1C (MDC1C). The methods of the present disclosure may also be applied in any disease or disorder associated with metabolism of ribitol and/or any disease or disorder for which ribitol is therapeutically effective.

The methods of this disclosure can be used to treat non-muscular dystrophy diseases for which restoration of and/or enhanced glycosylation of αDG would be beneficial and/or therapeutic. Thus, in some embodiments, the methods described herein may be used to treat other dystrophies which are associated with, or caused by, aberrant glycosylation of αDG.

Examples of diseases that may be treated in accordance with the methods described herein include, without limitation, Fukuyama Congenital Muscular Dystrophy (FCMD), Muscle-Eye-Brain (MEB) disease, Walker-Warburg syndrome (WWS), LGMD 2I/LGMD R9, FKRP-related Congenital Muscular Dystrophy Type 1C (MCD1C), Limb-Girdle Muscular Dystrophy type 2M (LGMD2M), Limb-Girdle Muscular Dystrophy type 2U (LGMD2U), and non-typed Limb Girdle Muscular Dystrophy (LGMD).

WWS, MEB and FCMD have common clinical findings, including brain malformations and muscular dystrophy (Martin, Nat Clin Pract Neurol., 2006; 2(4):222-230).

FCMD is also known as Fukuyama Syndrome and is caused by mutations in the FCMD or FKTN gene. This gene encodes fukutin, a putative glycosyltransferase. Fukuyama Syndrome patients present with early-onset (before 8 months of age) generalized symmetric weakness and hypotonia, delayed motor development, and elevated creatine kinase activity. Some patients also suffer from mental and speech retardation, seizures, as well as ocular abnormalities. Patients show a variable degree of clinical manifestations, including variability into the members of the same family. See Falsaperla et al., Ital J Pediatr. 2016; 42(1):78).

MEB is characterized by congenital muscular dystrophy, structural eye anomalies (usually congenital and may include severe myopia, glaucoma, optic nerve, and retinal hypoplasia), cerebral malformations, severe congenital weakness, inability to walk, spasticity, motor deterioration, mental retardation. The grade of severity of each organ affected is quite variable MEB is inherited in autosomal recessive pattern and is associated with mutations in the gene at 1p34-p32 that codifies POMGnT1, a glycosyl transferase, but may involve different genes such as POMGnTI, FKRP, Fukutin, ISPD, TMEM5. See Falsaperla et al., Ital J Pediatr. 2016; 42(1):78).

WWS is genetically heterogeneous and involves the POMT1, POMT2, and less frequently POMGnT1, FKRP, Fukutin, and LARGE genes. It is inherited in an autosomal-recessive fashion. Symptoms and signs are present at birth, and occasionally can be detected prenatally. Most of the affected children do not survive beyond the first years of life. WWS presents with generalized hypotonia, severe congenital muscular dystrophy, brain malformation (including lissencephaly type I with cobblestone cortex, obstructive hydrocephalus, neuronal heterotopias, corpus callosum agenesis, fusion of the hemispheres, and white matter hypomyelination), developmental delay with mental retardation). Eye anomalies, including anterior eye anomalies (cataracts, shallow anterior chamber, microcornea and microphthalmia, and lens defects) and posterior eye anomalies (retinal detachment or dysplasia, hypoplasia or atrophy of the optic nerve and macula and coloboma) may also be present, and some patients additionally suffer from facial dysmorphism and cleft lip or palate. Patients often show elevated creatine kinase, and altered α-dystroglycan. See Vaj sat and Schachter, Orphanet J Rare Dis., 2006; 1:29; see Falsaperla et al., Ital J Pediatr. 2016; 42(1):78).

MDC1C manifests in the first few weeks of life with CMD and marked increase of creatine kinase, but patients may present with normal intelligence and normal brain structures on brain imaging. Later, (in the young adult age), MDC1C progresses to include heart involvement, severe muscle hypertrophy and weakness, and severe respiratory failure. MDC1C also includes clinical features of CMD/LGMD involving different genes (FKRP, Fukutin, ISPD, GMPPB), e.g., early onset weakness and early onset LGMD without brain involvement and cardiomyopathy. See Falsaperla et al., Ital J Pediatr. 2016; 42(1):78).

Other types of CMDs belonging to alpha-dystroglycan related dystrophies include Congenital muscular dystrophy with partial merosin deficiency (MCD1B), which manifests with variable deficiency of the glycosylated aDG epitope and secondary laminin alpha 2 deficiency and proximal limb girdle weakness, muscle hypertrophy, particularly in the calf, and early respiratory failure, as well as LARGE related CMD (MDC1D), which shares clinical features of MEB and/or WWS and may present with mental retardation, severe generalized muscle weakness, and increased level of creatine kinase. See Martin, Nat Clin Pract Neurol., 2006; 2(4):222-230; see Falsaperla et al., Ital J Pediatr. 2016; 42(1):78).

The relationship between gene mutations and clinical manifestation is variable. Thus, for example, mutations in FKRP were initially not associated with no brain involvement, but FKRP V405L and A455D mutations have been linked to brain abnormalities including mental retardation, microcephaly and cerebellar cysts. Other mutations in this gene present as MEB or WWS. By contrast, homozygous L276I mutations cause LGMD2I, which is milder in presentation than MDC1C. In addition, it likely that all of these disorders are modulated by secondary genetic factors. See Martin, Nat Clin Pract Neurol., 2006; 2(4):222-230. According to some embodiments of the present invention, the methods of the present invention provide therapeutically effective blood plasma levels of ribitol for treating a disease or disorder. Blood plasma levels of ribitol may be expressed using pharmacokinetic (PK) parameters that are known to those skilled in the art, such as steady state plasma levels, AUC, Cₘₐₓ, and Gₘᵢₙ. Throughout the present disclosure pharmacokinetic parameters are described in terms of providing a steady state plasma level of a particular PK parameter (such as steady state plasma Cₘₐₓ, steady state AUC, etc.). However, the present disclosure contemplates embodiments where the steady state PK parameters that are expressed herein are average values from a patient population (such as a mean value). Thus, the following description of pharmacokinetic parameters describes mean steady state PK parameter values as well values from an individual patient. Unless otherwise specified, all PK parameters described herein are provided as steady state values.

In additional embodiments, the present disclosure provides a method of treating or inhibiting the development of muscle weakness in a subject, comprising administering to the subject a composition comprising an effective amount of ribitol, thereby treating or inhibiting the development of muscle weakness, e.g., muscle weakness which limits or slows daily activity of the subject.

The present disclosure further provides a method of treating a disorder associated with a defect in glycosylation of αDG, comprising administering to a subject that has or is suspected of having a disorder associated with a defect in glycosylation of αDG an effective amount of ribitol. A subject can be suspected of having a defect in glycosylation of αDG if the subject has muscle weakness even in cases where genetic and biochemical analyses of the subject have failed to identify a causative gene defect.

In additional embodiments, the present disclosure provides a method of treating a disorder associated with muscle weakness, comprising administering to a subject that has or is suspected of having or developing a disorder associated with muscle weakness an effective amount of ribitol. Muscle weakness can imply that a subject is not able to perform the daily activities that a normal person of similar gender, age, and other conditions would be expected to be capable of performing. An example is the loss of or lack of ability to climb stairs, run, or hold an object for an extended period.

Further provided herein is a method of treating a disorder associated with a defect in glycosylation of αDG caused by a mutation in the FKRP gene, comprising administering to a subject that has or is suspected of having a mutation in the FKRP gene an effective amount of ribitol. A mutation in an FKRP gene can be identified by, e.g., genetic analysis of the nucleic acid of a subject.

In some embodiments, an active compound or agent for use in the compositions and methods described herein can be ribitol.

In further embodiments, the methods of the disclosure comprise, in place of ribitol, administering a ribitol derivative or analog. The ribitol derivative may be, e.g., a tri-acetylated ribitol; per-acetylated ribitol, ribose; a phosphorylated ribitol (e.g., ribose-5-P); a nucleotide form of ribitol (e.g., a nucleotide-alditol having cytosine or other bases as the nucleobase with 1, 2, or 3 phosphate groups and ribitol as the alditol portion, such as CDP-ribitol or CDP-ribitol-OAc2); or a combination thereof.

Further aspects of this disclosure include the use of ribitol in the preparation of a medicament for carrying out the methods disclosed herein.

In some embodiments, administration of ribitol may be by any suitable route, including but not limited to intrathecal injection, subcutaneous, cutaneous, intravenous, intraperitoneal, intramuscular injection, intra-arterial, intratumoral or any intratissue injection, nasal, oral, sublingual, or by inhalation.

In some embodiments, ribitol is provided as a solid pharmaceutical composition, e.g., a tablet, capsule, or powder. The pharmaceutical composition may be lyophilized. Alternatively, the ribitol may be provided as solid (e.g., a powder) for reconstitution in a solution as a liquid pharmaceutical composition.

In some embodiments, encapsulated or compressed ribitol composition can be coated with a suitable film coat, erodible outer layer composition, mucoadhesive outer layer composition, or any combination thereof.

In some embodiments, the erodible outer layer composition can comprise a cellulosic polymer (e.g., HPMC, EC), vinylpyrrolidone-based polymer (e.g., PVP), polyethylene-based polymers (e.g., PEO, PEG), or combinations thereof. In some embodiments, the erodible outer layer composition can comprise hydroxypropyl methylcellulose (HMPC), ethyl cellulose, poly(ethylene oxide) (PEO), or any combination thereof.

In some embodiments, the mucoadhesive outer layer composition can comprise a carbohydrate polymer.

In some embodiments, the ribitol composition is a solution. In some embodiments, the ribitol composition is a powder. For example, the ribitol may be powder for oral administration, supplied in a sachet.

In some embodiments, the ribitol composition can further comprise pharmaceutically acceptable excipients, diluents, and/or carriers, including, but not limited to glucose, polyethylene glycol (PEG) (which in some embodiments can have a molecular weight in a range of about 200 to about 500), glycerin, water, substantially pure water, saline, or any combination thereof. Ribitol can be mixed or combined with any substance for improved delivery, absorption, etc.

In some embodiments, the therapeutically effective amount of ribitol is administered at a dose of at least about 0.5 g per day (g/day), at least about 1 g/day, at least about 2 g/day, at least about 3 g/day, at least about 4 g/day, at least about 5 g/day, at least about 7.5 g/day, at least about 10 g/day, at least about 12.5 g/day, at least about 15 g/day, at least about 20 g/day, at least about 25 g/day, at least about 30 g/day, at least about 35 g/day, at least about 40 g/day, at least about 45 g/day, at least about 50 g/day, at least about 55 g/day, at least about 60 g/day, at least about 70 g/day, at least about 80 g/day, at least about 90 g/day, at least about 100 g/day, at least about 110 g/day, at least about 120 g/day, at least about 130 g/day, at least about 140 g/day, at least about 150 g/day, at least about 160 g/day, at least about 170 g/day, at least about 180 g/day, at least about 190 g/day, at least about 200 g/day, at least about 210 g/day, or greater.

In some embodiments, the therapeutically effective amount of ribitol is administered at a dose of at most about 0.5 g/day, at most about 1 g/day, at most about 2 g/day, at most about 3 g/day, at most about 4 g/day, at most about 5 g/day, at most about 7.5 g/day, at most about 10 g/day, at most about 12.5 g/day, at most about 15 g/day, at most about 20 g/day, at most about 25 g/day, at most about 30 g/day, at most about 35 g/day, at most about 40 g/day, at most about 45 g/day, at most about 50 g/day, at most about 55 g/day, at most about 60 g/day, at most about 70 g/day, at most about 80 g/day, at most about 90 g/day, at most about 100 g/day, at most about 110 g/day, at most about 120 g/day, at most about 130 g/day, at most about 140 g/day, at most about 150 g/day, at most about 160 g/day, at most about 170 g/day, at most about 180 g/day, at most about 190 g/day, at most about 200 g/day, at most about 210 g/day, or less.

In some embodiments, the therapeutically effective amount of ribitol is administered at a dose of about 0.5 g/day to about 1 g/day, about 1 g/day to about 2 g/day, about 2 g/day to about 3 g/day, about 3 g/day to about 4 g/day, about 4 g/day to about 5 g/day, about 5 g/day to about 7.5 g/day, about 7.5 g/day to about 10 g/day, about 10 g/day to about 12.5 g/day, about 10 g/day to about 15 g/day, about 15 g/day to about 20 g/day, about 20 g/day to about 25 g/day, about 25 g/day to about 30 g/day, about 30 g/day to about 35 g/day, about 35 g/day to about 40 g/day, about 40 g/day to about 45 g/day, about 45 g/day to about 50 g/day, about 50 g/day to about 55 g/day, about 55 g/day to about 60 g/day, about 60 g/day to about 65 g/day, about 65 g/day to about 70 g/day, about 70 g/day to about 75 g/day, about 75 g/day to about 80 g/day, about 80 g/day to about 85 g/day, about 85 g/day to about 90 g/day, about 90 g/day to about 95 g/day, about 95 g/day to about 100 g/day, or any useful range therein.

In some embodiments, the therapeutically effective amount of ribitol is administered at a dose of about 0.5 g/day to about 2 g/day, about 2 g/day to about 4 g/day, about 4 g/day to about 7.5 g/day, 7.5 g/day to 12.5 g/day, 10 g/day to 15 g/day, 12 g/day to 22 g/day, 15 g/day to 25 g/day, 20 g/day to 30 g/day, 25 g/day to 35 g/day, 30 g/day to 40 g/day, 35 g/day to 45 g/day, 40 g/day to 50 g/day, 45 g/day to 55 g/day, 50 g/day to 60 g/day, 55 g/day to 65 g/day, 60 g/day to 70 g/day, 65 g/day to 75 g/day, 70 g/day to 80 g/day, 75 g/day to 85 g/day, 80 g/day to about 90 g/day, about 85 g/day to about 95 g/day, about 90 g/day to about 100 g/day, or any useful range therein.

In some embodiments, the therapeutically effective amount of ribitol is administered at a dose of about 0.5 g/day to about 4 g/day, about 4 g/day to about 12.5 g/day, about 10 g/day to about 15 g/day, about 12.5 g/day to about 17.5 g/day, about 15 g/day to about 20 g/day, about 17.5 g/day to about 22.5 g/day, about 20 g/day to about 25 g/day, about 22.5 g/day to about 27.5 g/day, about 25 g/day to about 30 g/day, about 27.5 g/day to about 32.5 g/day, or about 30 g/day to about 35 g/day, or any useful range therein.

In some embodiments, the therapeutically effective amount of ribitol is administered at a dose of about 0.5 g/day, about 1 g/day, about 1.5 g/day, about 2 g/day, about 3 g/day, about 4 g/day, about 5 g/day, about 6 g/day, about 7.5 g/day, about 10 g/day, about 12 g/day, about 12.5 g/day, or about 15 g/day.

In some embodiments, the therapeutically effective amount of ribitol is administered at a dose of about 0.5 g/day to about 1 g/day, about 0.5 g/day to about 1.5 g/day, about 0.5 g/day to about 2 g/day, about 0.5 g/day to about 3 g/day, about 0.5 g/day to about 4 g/day, about 0.5 g/day to about 5 g/day, about 0.5 g/day to about 6 g/day, about 0.5 g/day to about 7.5 g/day, about 0.5 g/day to about 10 g/day, about 0.5 g/day to about 12 g/day, about 0.5 g/day to about 12.5 g/day, about 0.5 g/day to about 15 g/day, about 0.5 g/day to about 20 g/day, about 0.5 g/day to about 25 g/day, about 0.5 g/day to about 30 g/day, about 0.5 g/day to about 40 g/day, about 0.5 g/day to about 50 g/day, about 0.5 g/day to about 60 g/day, about 0.5 g/day to about 70 g/day, about 0.5 g/day to about 80 g/day, about 0.5 g/day to about 90 g/day, or about 0.5 g/day to about 100 g/day, or any useful range therein.

In some embodiments, the therapeutically effective amount of ribitol is administered at a dose of about 1 g/day to about 1.5 g/day, about 1 g/day to about 2 g/day, about 1 g/day to about 3 g/day, about 1 g/day to about 4 g/day, about 1 g/day to about 5 g/day, about 1 g/day to about 6 g/day, about 1 g/day to about 7.5 g/day, about 1 g/day to about 10 g/day, about 1 g/day to about 12 g/day, about 1 g/day to about 12.5 g/day, about 1 g/day to about 15 g/day, about 1 g/day to about 20 g/day, about 1 g/day to about 25 g/day, about 1 g/day to about 30 g/day, about 1 g/day to about 40 g/day, about 1 g/day to about 50 g/day, about 1 g/day to about 60 g/day, about 1 g/day to about 70 g/day, about 1 g/day to about 80 g/day, about 1 g/day to about 90 g/day, or about 1 g/day to about 100 g/day, or any useful range therein.

In some embodiments, the therapeutically effective amount of ribitol is administered at a dose of about 2 g/day to about 3 g/day, about 2 g/day to about 4 g/day, about 2 g/day to about 5 g/day, about 2 g/day to about 6 g/day, about 2 g/day to about 7.5 g/day, about 2 g/day to about 10 g/day, about 2 g/day to about 12 g/day, about 2 g/day to about 12.5 g/day, about 2 g/day to about 15 g/day, about 2 g/day to about 20 g/day, about 2 g/day to about 25 g/day, about 2 g/day to about 30 g/day, about 2 g/day to about 35 g/day, about 2 g/day to about 40 g/day, about 2 g/day to about 50 g/day, about 2 g/day to about 60 g/day, about 2 g/day to about 70 g/day, about 2 g/day to about 80 g/day, about 2 g/day to about 90 g/day, or about 2 g/day to about 100 g/day, or any useful range therein.

In some embodiments, the therapeutically effective amount of ribitol is administered at a dose of about 3 g/day to about 4 g/day, about 3 g/day to about 5 g/day, about 3 g/day to about 6 g/day, about 3 g/day to about 7.5 g/day, about 3 g/day to about 10 g/day, about 3 g/day to about 12 g/day, about 3 g/day to about 12.5 g/day, about 3 g/day to about 15 g/day, about 3 g/day to about 20 g/day, about 3 g/day to about 25 g/day, about 3 g/day to about 30 g/day, about 3 g/day to about 35 g/day, about 3 g/day to about 40 g/day, about 3 g/day to about 50 g/day, about 3 g/day to about 60 g/day, about 3 g/day to about 70 g/day, about 3 g/day to about 80 g/day, about 3 g/day to about 90 g/day, or about 3 g/day to about 100 g/day, or any useful range therein.

In some embodiments, the therapeutically effective amount of ribitol is administered at a dose of about 4 g/day to about 5 g/day, about 4 g/day to about 6 g/day, about 4 g/day to about 7.5 g/day, about 4 g/day to about 10 g/day, about 4 g/day to about 12 g/day, about 4 g/day to about 12.5 g/day, about 4 g/day to about 15 g/day, about 4 g/day to about 20 g/day, about 4 g/day to about 25 g/day, about 4 g/day to about 30 g/day, about 4 g/day to about 35 g/day, about 4 g/day to about 40 g/day, about 4 g/day to about 50 about g/day, about 4 g/day to about 60 g/day, about 4 g/day to about 70 g/day, about 4 g/day to about 80 g/day, about 4 g/day to about 90 g/day, or about 4 g/day to about 100 g/day, or any useful range therein.

In some embodiments, the therapeutically effective amount of ribitol is administered at a dose of about 5 g/day to about 6 g/day, about 5 g/day to about 7.5 g/day, about 5 g/day to about 10 g/day, about 5 g/day to about 12 g/day, about 5 g/day to about 12.5 g/day, about 5 g/day to about 15 g/day, about 5 g/day to about 20 g/day, about 5 g/day to about 30 g/day, about 5 g/day to about 40 g/day, about 5 g/day to about 50 g/day, about 5 g/day to about 60 g/day, about 5 g/day to about 70 g/day, about 5 g/day to about 80 g/day, about 5 g/day to about 90 g/day, or about 5 g/day to about 100 g/day, or any useful range therein.

In some embodiments, the therapeutically effective amount of ribitol is administered at a dose of about 7.5 g/day to about 10 g/day, about 7.5 g/day to about 12 g/day, about 7.5 g/day to about 12.5 g/day, about 7.5 g/day to about 15 g/day, about 7.5 g/day to about 20 g/day, about 7.5 g/day to about 30 g/day, about 7.5 g/day to about 40 g/day, about 7.5 g/day to about 50 g/day, about 7.5 g/day to about 60 g/day, about 7.5 g/day to about 70 g/day, about 7.5 g/day to about 80 g/day, about 7.5 g/day to about 90 g/day, or about 7.5 g/day to about 100 g/day, or any useful range therein.

In some embodiments, the therapeutically effective amount of ribitol is administered at a dose of about 10 g/day to about 12 g/day, about 10 g/day to about 12.5 g/day, about 10 g/day to about 15 g/day, about 10 g/day to about 20 g/day, about 10 g/day to about 25 g/day, about 10 g/day to about 30 g/day, about 10 g/day to about 40 g/day, about 10 g/day to about 50 g/day, about 10 g/day to about 60 g/day, about 10 g/day to about 70 g/day, about 10 g/day to about 80 g/day, about 10 g/day to about 90 g/day, or about 10 g/day to about 100 g/day, or any useful range therein.

In some embodiments, the therapeutically effective amount of ribitol is administered at a dose of about 12.5 g/day to about 15 g/day, about 12.5 g/day to about 20 g/day, about 12.5 g/day to about 25 g/day, about 12.5 g/day to about 30 g/day, about 12.5 g/day to about 35 g/day, about 12.5 g/day to about 40 g/day, about 12.5 g/day to about 50 g/day, about 12.5 g/day to about 60 g/day, about 12.5 g/day to about 70 g/day, about 12.5 g/day to about 80 g/day, about 12.5 g/day to about 90 g/day, or about 12.5 g/day to about 100 g/day, or any useful range therein.

In some embodiments, the therapeutically effective amount of ribitol is administered at a dose about 3 g to about 12 g twice daily (BID). In some embodiments, the therapeutically effective amount of ribitol is administered at a dose of at least about 3 g BID. In some embodiments, the therapeutically effective amount of ribitol is administered at a dose of about 12 g BID.

In some embodiments, the therapeutically effective amount of ribitol is administered at least once daily, at least twice daily, at least three times daily, at least four times daily, at least five times daily, or at least six times daily. In preferred embodiments, the therapeutically effective amount of ribitol is administered twice daily (“BID”). In some embodiments, the effective amount of ribitol is administered about every 12 hours (“Q12 hours”).

The therapeutically effective dose of ribitol may be adjusted based on characteristics of the patient being treated, for example, age, body weight, body surface area, and/or expression levels of metabolic enzymes. Examples of dosing regimens for ribitol in adults are set forth in Table 1 below. Examples of dosing regimens for ribitol in children aged 12-18 years are set forth in Table 2. Examples of dosing regimens for ribitol in children aged 2-5 years are set forth in Table 3. In some embodiments, a child aged 5-12 years may be treated with a dosing regimen set forth in Table 2.

Without wishing to be bound by theory, the dosing regimens shown in Table 1-Table 3 are expected to place patients within the ‘efficacious’ range of an Area under the Curve of 0-24 (“AUC₀₋₂₄”). While BID dosing or Q12 hour doing is preferable in many cases, single dose per day, and even lower frequencies are possible and may sometimes be advantageous.

TABLE 1
Exemplary Dosing Regimens for Ribitol in Adults

| Body Weight Range | Possible Dosing Schedule |
|---|---|
| All adults, regardless of weight | 12 g BID/Q12 hours |
| All patients, regardless of weight | 15 g BID/Q12 hours |
| | 18 g BID/Q12 hours |
| >70 kg | 12 g BID/Q12 hours |
| | 15 g BID/Q12 hours |
| | 18 g BID/Q12 hours |
| >65 kg | 9 g BID/Q12 hours |
| | 12 g BID/Q12 hours |
| | 15 g BID/Q12 hours |
| | 18 g BID/Q12 hours |
| >60 kg | 9 g BID/Q12 hours |
| | 12 g BID/Q12 hours |
| | 15 g BID/Q12 hours |
| | 18 g BID/Q12 hours |
| >55 kg | 9 g BID/Q12 hours |
| | 12 g BID/Q12 hours |
| | 15 g BID/Q12 hours |
| | 18 g BID/Q12 hours |
| >50 kg | 9 g BID/Q12 hours |
| | 12 g BID/Q12 hours |
| | 15 g BID/Q12 hours |
| | 18 g BID/Q12 hours |
| >45 kg | 9 g BID/Q12 hours |
| | 12 g BID/Q12 hours |
| | 15 g BID/Q12 hours |
| | 18 g BID/Q12 hours |
| >40 kg | 6 g BID/Q12 hours |
| | 9 g BID/Q12 hours |
| | 12 g BID/Q12 hours |
| | 15 g BID/Q12 hours |
| | 18 g BID/Q12 hours |
| >35 kg | 6 g BID/Q12 hours |
| | 9 g BID/Q12 hours |
| | 12 g BID/Q12 hours |
| | 15 g BID/Q12 hours |
| | 18 g BID/Q12 hours |
| >30 kg | 6 g BID/Q12 hours |
| | 9 g BID/Q12 hours |
| | 12 g BID/Q12 hours |
| | 15 g BID/Q12 hours |
| | 18 g BID/Q12 hours |

TABLE 2
Exemplary Dosing Regimens for Ribitol
in Children aged 12-18 years.

| Body Weight Range | Possible Dosing Schedule |
|---|---|
| All Children, regardless of weight | 12 g BID/Q12 hours |
| All Children 12-18, regardless of weight | 15 g BID/Q12 hours |
| All patients, regardless of weight | 18 g BID/Q12 hours |
| >70 kg | 12 g BID/Q12 hours |
| | 15 g BID/Q12 hours |
| | 18 g BID/Q12 hours |
| >65 kg | 9 g BID/Q12 hours |
| | 12 g BID/Q12 hours |
| | 15 g BID/Q12 hours |
| | 18 g BID/Q12 hours |
| >60 kg | 9 g BID/Q12 hours |
| | 12 g BID/Q12 hours |
| | 15 g BID/Q12 hours |
| | 18 g BID/Q12 hours |
| >55 kg | 9 g BID/Q12 hours |
| | 12 g BID/Q12 hours |
| | 15 g BID/Q12 hours |
| | 18 g BID/Q12 hours |
| >50 kg | 9 g BID/Q12 hours |
| | 12 g BID/Q12 hours |
| | 15 g BID/Q12 hours |
| | 18 g BID/Q12 hours |
| >45 kg | 9 g BID/Q12 hours |
| | 12 g BID/Q12 hours |
| | 15 g BID/Q12 hours |
| | 18 g BID/Q12 hours |
| >40 kg | 6 g BID/Q12 hours |
| | 9 g BID/Q12 hours |
| | 12 g BID/Q12 hours |
| | 15 g BID/Q12 hours |
| | 18 g BID/Q12 hours |
| >35 kg | 6 g BID/Q12 hours |
| | 9 g BID/Q12 hours |
| | 12 g BID/Q12 hours |
| | 15 g BID/Q12 hours |
| | 18 g BID/Q12 hours |
| >30 kg | 6 g BID/Q12 hours |
| | 9 g BID/Q12 hours |
| | 12 g BID/Q12 hours |
| | 15 g BID/Q12 hours |
| | 18 g BID/Q12 hours |
| >25 kg | 6 g BID/Q12 hours |
| | 9 g BID/Q12 hours |
| | 12 g BID/Q12 hours |
| | 15 g BID/Q12 hours |
| | 18 g BID/Q12 hours |
| >20 kg | 3 g BID/Q12 hours |
| | 6 g BID/Q12 hours |
| | 9 g BID/Q12 hours |
| | 12 g BID/Q12 hours |
| | 15 g BID/Q12 hours |
| | 18 g BID/Q12 hours |

TABLE 3
Exemplary Dosing Regimens for Ribitol
in Children aged 2-5 years.

| Body Weight Range | Possible Dosing Schedule |
|---|---|
| All Children, regardless of weight | 12 g BID/Q12 hours |
| All Children 2-S years of age, | 15 g BID/Q12 hours |
| regardless of weight | 18 g BID/Q12 hours |
| All patients, regardless of weight | |
| >70 kg | 12 g BID/Q12 hours |
| | 15 g BID/Q12 hours |
| | 18 g BID/Q12 hours |
| >65 kg | 9 g BID/Q12 hours |
| | 12 g BID/Q12 hours |
| | 15 g BID/Q12 hours |
| | 18 g BID/Q12 hours |
| >60 kg | 9 g BID/Q12 hours |
| | 12 g BID/Q12 hours |
| | 15 g BID/Q12 hours |
| | 18 g BID/Q12 hours |
| >55 kg | 9 g BID/Q12 hours |
| | 12 g BID/Q12 hours |
| | 15 g BID/Q12 hours |
| | 18 g BID/Q12 hours |
| >50 kg | 9 g BID/Q12 hours |
| | 12 g BID/Q12 hours |
| | 15 g BID/Q12 hours |
| | 18 g BID/Q12 hours |
| >45 kg | 9 g BID/Q12 hours |
| | 12 g BID/Q12 hours |
| | 15 g BID/Q12 hours |
| | 18 g BID/Q12 hours |
| >40 kg | 6 g BID/Q12 hours |
| | 9 g BID/Q12 hours |
| | 12 g BID/Q12 hours |
| | 15 g BID/Q12 hours |
| | 18 g BID/Q12 hours |
| >35 kg | 6 g BID/Q12 hours |
| | 9 g BID/Q12 hours |
| | 12 g BID/Q12 hours |
| | 15 g BID/Q12 hours |
| | 18 g BID/Q12 hours |
| >30 kg | 6 g BID/Q12 hours |
| | 9 g BID/Q12 hours |
| | 12 g BID/Q12 hours |
| | 15 g BID/Q12 hours |
| | 18 g BID/Q12 hours |
| >25 kg | 6 g BID/Q12 hours |
| | 9 g BID/Q12 hours |
| | 12 g BID/Q12 hours |
| | 15 g BID/Q12 hours |
| | 18 g BID/Q12 hours |
| >20 kg | 3 g BID/Q12 hours |
| | 6 g BID/Q12 hours |
| | 9 g BID/Q12 hours |
| | 12 g BID/Q12 hours |
| | 15 g BID/Q12 hours |
| | 18 g BID/Q12 hours |
| >17.5 kg | 3 g BID/Q12 hours |
| | 6 g BID/Q12 hours |
| | 9 g BID/Q12 hours |
| | 12 g BID/Q12 hours |
| | 15 g BID/Q12 hours |
| | 18 g BID/Q12 hours |
| >15 kg | 3 g BID/Q12 hours |
| | 6 g BID/Q12 hours |
| | 9 g BID/Q12 hours |
| | 12 g BID/Q12 hours |
| | 15 g BID/Q12 hours |
| | 18 g BID/Q12 hours |
| >12.5 kg | 3 g BID/Q12 hours |
| | 6 g BID/Q12 hours |
| | 9 g BID/Q12 hours |
| | 12 g BID/Q12 hours |
| | 15 g BID/Q12 hours |
| | 18 g BID/Q12 hours |
| >10 kg | 3 g BID/Q12 hours |
| | 6 g BID/Q12 hours |
| | 9 g BID/Q12 hours |
| | 12 g BID/Q12 hours |
| | 15 g BID/Q12 hours |
| | 18 g BID/Q12 hours |
| All children regardless of age | 0.5 g/kg BID/q12 hours |
| All children 2-5 years of age | 0.75 g/kg BID/q12 hours |
| All children 0-2 years of age | 1.0 g/kg BID/q12 hours |
| All children 0-5 years of age | 1.25 g/kg BID/q12 hours |
| All children 0-1 years of age | 1.5 g/kg BID/q12 hours |
| All children 1-5 years of age | 1.75 g/kg BID/q12 hours |
| All children 1-2 years of age | 2.0 g/kg BID/q 12 hours |
| | 2.25 g/kg BID/q12 hours |
| | 2.5 g/kg BID/q12 hours |
| | 3.0 g/kg BID/q12 hours |

In some embodiments, the therapeutically effective amount of ribitol is administered for at least one day, two days, three days, four days, five days, six days, seven days, eight days, nine days, ten days, eleven days, twelve days, thirteen days, two weeks, seventeen days, three weeks, twenty-five days, four weeks, five weeks, or six weeks.

In some embodiments, the therapeutically effective amount of ribitol is administered for at least one month, two months, three months, four months, five months, six months, seven months, eight months, nine months, ten months, eleven months, one year, eighteen months, two years, three years, four years, five years, six years, seven years, eight years, nine years, or ten years.

In some embodiments, the method comprises administering the dose of ribitol chronically, such as for at least six months.

In some embodiments, the maximum observed concentration (Cₘₐₓ) of ribitol is between about 50 μg/mL and about 2500 μg/mL.

In some embodiments, the Cₘₐₓ of ribitol is at least about 50 μg/mL, at least about 75 μg/mL, at least about 100 μg/mL, at least about 150 μg/mL, at least about 200 μg/mL, at least about 300 μg/mL, at least about 400 μg/mL, at least about 500 μg/mL, at least about 600 μg/mL, at least about 700 μg/mL, at least about 800 μg/mL, at least about 900 μg/mL, at least about 1000 μg/mL, at least about 1100 μg/mL, at least about 1200 μg/mL, at least about 1300 μg/mL, at least about 1400 μg/mL, at least about 1500 μg/mL, at least about 1600 μg/mL, at least about 1700 μg/mL, at least about 1800 μg/mL, at least about 1900 μg/mL, at least about 2000 μg/mL, at least about 2100 μg/mL, at least about 2200 μg/mL, at least about 2300 μg/mL, at least about 2400 μg/mL, at least about 2500 μg/mL, or greater.

In some embodiments, the area under the plasma concentration-time curve (AUC₀₋₂₄) at steady-state for ribitol is between about 100 (μg·h)/mL and about 8000 (μg·h)/mL. It will be apparent to a person of skill in the art that the serum concentration and the plasma concentration of ribitol are related and either one may be used to generate a steady-state AUC₀₋₂₄.

In some embodiments, the AUC₀₋₂₄ for ribitol is at least about 100 (μg·h)/mL, at least about 200 (μg·h)/mL, at least about 300 (μg·h)/mL, at least about 400 (μg·h)/mL, at least about 500 (μg·h)/mL, at least about 600 (μg·h)/mL, at least about 700 (μg·h)/mL, at least about 800 (μg·h)/mL, at least about 900 (μg·h)/mL, at least about 1000 (μg·h)/mL, at least about 1100 (μg·h)/mL, at least about 1200 (μg·h)/mL, at least about 1300 (μg·h)/mL, at least about 1400 (μg·h)/mL, at least about 1500 (μg·h)/mL, at least about 1600 (μg·h)/mL, at least about 1700 (μg·h)/mL, at least about 1800 (μg·h)/mL, at least about 1900 (μg·h)/mL, at least about 2000 (μg·h)/mL, at least about 2100 (μg·h)/mL, at least about 2200 (μg·h)/mL, at least about 2300 (μg·h)/mL, at least about 2400 (μg·h)/mL, at least about 2500 (μg·h)/mL, at least about 2600 (μg·h)/mL, at least about 2700 (μg·h)/mL, at least about 2800 (μg·h)/mL, at least about 2900 (μg·h)/mL, at least about 3000 (μg·h)/mL, at least about 3500 (μg·h)/mL, at least about 4000 (μg·h)/mL, at least about 4500 (μg·h)/mL, at least about 5000 (μg·h)/mL, at least about 5500 (μg·h)/mL, at least about 6000 (μg·h)/mL, at least about 6500 (μg·h)/mL, at least about 7000 (μg·h)/mL, at least about 7500 (μg·h)/mL, or at least about 8000 (μg·h)/mL. In a preferred embodiment, the AUC₀₋₂₄ for ribitol is at least about 182 (μg·h)/mL,

In some embodiments, the AUC₀₋₂₄ for ribitol is about 100 (μg·h)/mL to about 200 (μg·h)/mL, about 200 (μg·h)/mL to about 300 (μg·h)/mL, about 300 (μg·h)/mL to about 400 (μg·h)/mL, about 400 (μg·h)/mL to about 500 (μg·h)/mL, about 500 (μg·h)/mL to about 600 (μg·h)/mL, about 600 (μg·h)/mL to about 700 (μg·h)/mL, about 700 (μg·h)/mL to about 800 (μg·h)/mL, about 800 (μg·h)/mL to about 900 (μg·h)/mL, about 900 (μg·h)/mL to about 1000 (μg·h)/mL, about 1000 (μg·h)/mL to about 1100 (μg·h)/mL, about 1100 (μg·h)/mL to about 1200 (μg·h)/mL, about 1200 (μg·h)/mL to about 1300 (μg·h)/mL, about 1300 (μg·h)/mL to about 1400 (μg·h)/mL, about 1400 (μg·h)/mL to about 1500 (μg·h)/mL, about 1500 (μg·h)/mL to about 1600 (μg·h)/mL, about 1600 (μg·h)/mL to about 1700 (μg·h)/mL, about 1700 (μg·h)/mL to about 1800 (μg·h)/mL, about 1800 (μg·h)/mL to about 1900 (μg·h)/mL, about 1900 (μg·h)/mL to about 2000 (μg·h)/mL, about 2000 (μg·h)/mL to about 2100 (μg·h)/mL, about 2100 (μg·h)/mL to about 2200 (μg·h)/mL, about 2200 (μg·h)/mL to about 2300 (μg·h)/mL, about 2300 (μg·h)/mL to about 2400 (μg·h)/mL, about 2400 (μg·h)/mL to about 2500 (μg·h)/mL, about 2500 (μg·h)/mL to about 2600 (μg·h)/mL, about 2600 (μg·h)/mL to about 2700 (μg·h)/mL, about 2700 (μg·h)/mL to about 2800 (μg·h)/mL, about 2800 (μg·h)/mL to about 2900 (μg·h)/mL, about 2900 (μg·h)/mL to about 3000 (μg·h)/mL, about 3000 (μg·h)/mL to about 3500 (μg·h)/mL, about 3500 (μg·h)/mL to about 4000 (μg·h)/mL, about 4000 (μg·h)/mL to about 4500 (μg·h)/mL, about 4500 (μg·h)/mL to about 5000 (μg·h)/mL, about 5000 (μg·h)/mL to about 5500 (μg·h)/mL, about 5500 (μg·h)/mL, to about 6000 (μg·h)/mL, about 6000 (μg·h)/mL to about 6500 (μg·h)/mL, about 6500 (μg·h)/mL to about 7000 (μg·h)/mL, about 7000 (μg·h)/mL to about 7500 (μg·h)/mL, or about 7500 (μg·h)/mL to about 8000 (μg·h)/mL.

In some embodiments, the therapeutically effective amount of ribitol is between about 0.2 g/mL and about 10 g/mL.

In some embodiments, the therapeutically effective amount of ribitol is at least about 0.01 g/mL, at least about 0.05 g/mL, at least about 0.1 g/mL, at least about 0.2 g/mL, at least about 0.3 g/mL, at least about 0.4 g/mL, at least about 0.5 g/mL, at least about 0.6 g/mL, at least about 0.7 g/mL, at least about 0.8 g/mL, at least about 0.9 g/mL, at least about 1 g/mL, at least about 2 g/mL, at least about 3 g/mL, at least about 4 g/mL, at least about 5 g/mL, at least about 6 g/mL, at least about 7 g/mL, at least about 8 g/mL, at least about 9 g/mL, or at least about 10 g/mL.

In some embodiments of the present disclosure, the disclosure provides a unit dose of ribitol, which may comprise between about 0.5 g and about 50 g of ribitol. The unit dose may be provided in solid form (e.g., as dry powder sachet) or in solution. Prior to administration, the unit dose may be diluted into water, or another suitable diluent. The concentration of the solution may be between about 20 mg/mL and about 250 mg/mL. In some cases, a unit dose in liquid solution may have a total volume of about 25 mL, about 50 mL, about 75 mL, or about 100 mL.

In some embodiments of the present disclosure, a unit dose of ribitol comprises at least about 0.5 g, at least about 1 g, at least about 1.5 g, at least about 2 g, at least about 2.5 g, at least about 3 g, at least about 3.5 g, at least about 4 g, at least about 4.5 g, at least about 5 g, at least about 5.5 g, at least about 6 g, at least about 6.5 g, at least about 7 g, at least about 7.5 g, at least about 8 g, at least about 8.5 g, at least about 9 g, at least about 9.5 g, at least about 10 g, at least about 10.5 g, at least about 11 g, at least about 11.5 g, at least about 12 g, at least about 12.5 g, at least about 13 g, at least about 13.5 g, at least about 14 g, at least about 14.5 g, at least about 15 g, at least about 16 g, at least about 18 g, at least about 20 g, at least about 22 g, at least about 24 g, at least about 26 g, at least about 28 g, at least about 30 g, at least about 33 g, at least about 36 g, at least about 39 g, at least about 42 g, at least about 45 g, at least about 47.5 g, or at least about 50 g.

In an aspect, the present disclosure provides a kit comprising a composition, e.g., for use in the treatment of a disease or disorder. In some embodiments, a kit of the present disclosure comprises a composition comprising a therapeutically effective amount of ribitol.

In some embodiments, a kit of the present disclosure comprises any number of the compositions and/or formulations of the present disclosure.

In some embodiments, a kit of the present disclosure comprises instructions for use in treating a disease or disorder.

The formulations disclosed herein include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces), and transdermal administration, although the most suitable route in any given case will depend on the nature and severity of the condition being treated and on the nature of the particular active compound which is being used.

The efficacy of a method of treatment described herein can be assessed by any suitable endpoint or measurement known in the art or described herein. Thus, treatment effect may be assessed with biomarkers including but not limited to measures of, αDG levels, αDG glycosylation levels, measures of matriglycan expression levels and amino terminus fragments levels of αDG, measures of markers of muscle damage (e.g., creatine kinase (CK), aldolase, troponins), measures of muscle performance (e.g., walk tests, grip strength), measures of cardiac function (e.g., echocardiography), measures of airway performance (e.g., plethysmography), and/or imaging based methods to assess muscle changes (e.g., magnetic resonance imaging (MRI)).

In some embodiments, a method of treatment described herein can result in a decrease in creatine kinase levels in a patient. Creatine kinase is an intracellular enzyme present in greatest amounts in skeletal muscle, myocardium, and brain; smaller amounts occur in other visceral tissues. It is generally used as a marker for muscle damage, and creatine kinase levels may be detected in the blood, serum, or plasma, for example, by measuring the rate of NADPH formation in the conversion of phosphocreatine and ADP to creatine and ATP. One unit of creatine kinase is defined as the amount of enzyme that will transfer 1.0 mmole of phosphate from phosphocreatine to ADP per minute at pH 6.0. See Cabaniss, in Clinical Methods: The History, Physical, and Laboratory Examinations. 3rd edition. Boston: Butterworths; 1990. Chapter 32. Assay kits to measure the creatine kinase activity in blood are commercially available. In some embodiments, a method of treatment described herein results in a decrease in creatine kinase levels of about 10% to about 20%, about 20% to about 30%, about 30% to about 40%, about 40% to about 50%, about 50% to about 60%, about 60% to about 70%, about 70% to about 80%, about 80% to about 90% or greater than about 90% compared to pre-treatment levels. In some embodiments, a method of treatment described herein results in creatine kinase levels in a patient returning to the normal range, which is about 26-192 U/L in women and 39-308 U/L in men.

In some embodiments, a method of treatment described herein results in an increase in αDG levels in a patient (see, e.g., Crowe et al., J Neuromuscul Dis. 2016 May 27; 3(2): 247-260). αDG levels in a patient may be assessed using any suitable method described herein or known in the art, including, for example, Enzyme-Linked Immunosorbent Assay (ELISA), Western Blotting and immunohistochemistry. In some embodiments, a method of treatment described herein results in an increase in αDG levels of about 1.1-fold to about 2-fold, about 2-fold to about 3-fold, about 3-fold to about 4-fold, about 4-fold to about 5-fold, about 5-fold to about 6-fold, about 6-fold to about 7-fold, about 7-fold to about 8-fold, about 8-fold to about 9-fold, about 9-fold to about 10-fold, about 10-fold to about 15-fold, about 15-fold to about 20-fold, about 20-fold to about 25-fold, about 25-fold to about 30-fold, about 30-fold to about 40-fold, about 40-fold to about 50-fold, about 50-fold to about 60-fold, about 60-fold to about 70-fold, about 80-fold to about 90-fold, or about 90-fold to about 100-fold compared to pre-treatment levels.

In some embodiments, a method of treatment described herein results in an increase in glycosylation of αDG. Glycosylation of αDG in a patient may be assessed using any suitable method described herein or known in the art, including, for example, Enzyme-Linked Immunosorbent Assay (ELISA), Western Blotting and immunohistochemistry. In some embodiments, a method of treatment described herein results in an increase in glycosylation of αDG of about 1.1-fold to about 2-fold, about 2-fold to about 3-fold, about 3-fold to about 4-fold, about 4-fold to about 5-fold, about 5-fold to about 6-fold, about 6-fold to about 7-fold, about 7-fold to about 8-fold, about 8-fold to about 9-fold, about 9-fold to about 10-fold, about 10-fold to about 15-fold, about 15-fold to about 20-fold, about 20-fold to about 25-fold, about 25-fold to about 30-fold, about 30-fold to about 40-fold, about 40-fold to about 50-fold, about 50-fold to about 60-fold, about 60-fold to about 70-fold, about 80-fold to about 90-fold, or about 90-fold to about 100-fold compared to pre-treatment levels.

In some embodiments, a method of treatment described herein results in an increase in the ratio of glycan to creatine in a patient. In some embodiments, the ratio increases by about 0.1 to about 0.2, about 0.2 to about 0.3, about 0.4 to about 0.5, about 0.5 to about 0.6, about 0.6 to about 0.7, or by about 0.7 to about 0.8. In some embodiments, the ratio returns to the normal value of about 0.9

In some embodiments, a method of treatment described herein results in an increase in matriglycan levels in the patient. Matriglycan levels may be measured using any suitable method described herein or known in the art, including, for example, Western Blotting and immunohistochemistry. In some embodiments, a method of treatment described herein results in an increase in matriglycan levels of about 1.1-fold to about 2-fold, about 2-fold to about 3-fold, about 3-fold to about 4-fold, about 4-fold to about 5-fold, about 5-fold to about 6-fold, about 6-fold to about 7-fold, about 7-fold to about 8-fold, about 8-fold to about 9-fold, about 9-fold to about 10-fold, about 10-fold to about 15-fold, about 15-fold to about 20-fold, about 20-fold to about 25-fold, about 25-fold to about 30-fold, about 30-fold to about 40-fold, about 40-fold to about 50-fold, about 50-fold to about 60-fold, about 60-fold to about 70-fold, about 80-fold to about 90-fold, or about 90-fold to about 100-fold compared to pre-treatment levels.

A method of treatment described herein may also be assessed using disease symptoms such as muscle fatigue and motor function, Activities of Daily Living (ADL).

An exemplary scale to assess muscle fatigue has been described, by Berard et al. Neuromuscular Disorders 15 (2005) 463-470. The scale comprises 32 items, in three dimensions: standing position and transfers, axial and proximal motor function, and distal motor function.

ADL scores have been described, see, e.g., Pettinato, et al., The Cerebellum (2021) 20:596-605. An exemplary ADL score comprises nine domains (speech, swallowing, ability to feed itself, dressing, sitting, walking, frequency of falls, selfhygiene and bladder function), each of which is measured on a scale from 0 (normal function) to 4 (severe functional disability). On such a scale, the maximum overall score is 36 indicating very severe functional disability.

In some embodiments, a method of treatment described herein results in a normalization of structural abnormalities in the eye or brain. Such changes can be assessed using, for example, CT scan or MRI.

Formulations suitable for oral administration may be presented in discrete units, such as capsules, cachets, sachets, stick packs, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations disclosed herein are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavored base, usually sucrose and acacia or tragacanth; and pastilles comprising the compound in an inert base such as gelatin and glycerin or sucrose and acacia.

Formulations of the present disclosure suitable for parenteral administration comprise sterile aqueous and non-aqueous injection solutions of the active compound(s), which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present disclosure, there is provided an injectable, stable, sterile composition comprising an active compound(s), or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

In addition to active compound(s), the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present disclosure may be lyophilized using techniques well known in the art.

In some embodiments, a therapeutic agent for use in the compositions and methods described herein can be ribitol.

In further embodiments, the methods of the disclosure comprise, in place of ribitol, administering a ribitol derivative or analog. The ribitol derivative may be, e.g., a tri-acetylated ribitol; per-acetylated ribitol, ribose; a phosphorylated ribitol (e.g., ribose-5-P); a nucleotide form of ribitol (e.g., a nucleotide-alditol having cytosine or other bases as the nucleobase with 1, 2 or 3 phosphate groups and ribitol as the alditol portion, such as CDP-ribitol or CDP-ribitol-OAc2); or a combination thereof.

In some embodiments, the disease or disorder is associated with a defect in Fukutin-related protein (FKRP). In some embodiments, the disease or disorder is associated with a defect in fukutin (FKTN). In additional embodiments, a subject has a mutation in a gene encoding fukutin (FKTN), fukutin-related protein (FKRP), or isoprenoid synthase domain-containing protein (ISPD) that causes a partial or complete loss-of-function in FKRP.

In some embodiments, the therapeutic agent of the present disclosure may improve and/or prevent one or more symptoms of disease, including but not limited to limb muscle weakness, e.g., with mild calf and thigh hypertrophy; decreased hip flexion and adduction; decreased knee flexion and ankle dorsiflexion; progressive muscle degeneration; fiber wasting; decreased matriglycan expression; infiltration and accumulation of fibrosis and/or fat in muscle tissues; loss of ambulation. Muscle weakness may involve the diaphragm with varying severity, leading to respiratory failure in a proportion of patients. Cardiac muscle is affected with the most severe and frequent presentation being dilated cardiomyopathy.

### ENUMERATED EMBODIMENTS

The disclosure provides the following enumerated embodiments:

Clause 1. Ribitol for use in a method of treating a disease or disorder associated with in a subject in need thereof, comprising administering a dose comprising an effective amount of ribitol, optionally an immediate-release dose and/or not an extended-release dose.

Clause 2. Ribitol for use in the method of clause 1, wherein the dose is administered at most four times daily.

Clause 3. Ribitol for use in the method of clause 2, wherein the dose is administered at most twice daily.

Clause 4. Ribitol for use in the method of clause 1, wherein the dose is administered three times daily.

Clause 5. Ribitol for use in the method of clause 3, wherein the dose is administered twice daily.

Clause 6. Ribitol for use in the method of clause 3, wherein the dose is administered once daily.

Clause 7. Ribitol for use in the method of any of clauses 1 to 5, wherein the method comprises administering at least 0.5 grams per day (g/day), at least 1 g/day, at least 2 g/day, at least 3 g/day, at least 4 g/day, at least 5 g/day, at least 7.5 g/day, at least 10 g/day, at least 12.5 g/day, or at least 15 g/day.

Clause 8. Ribitol for use in the method of any of clauses 1 to 6, wherein the method comprises administering at most 0.5 g/day, at most 1 g/day, at most 2 g/day, at most 3 g/day, at most 4 g/day, at most 5 g/day, at most 7.5 g/day, at most 10 g/day, at most 12.5 g/day, or at most 15 g/day.

Clause 9. Ribitol for use in the method of any of clauses 1 to 5, wherein the method comprises administering 0.5 g/day, 1 g/day, 1.5 g/day, 2 g/day, 3 g/day, 4 g/day, 5 g/day, 6 g/day, 7.5 g/day, 10 g/day, 12 g/day, 12.5 g/day, or 15 g/day.

Clause 10. Ribitol for use in the method of clause 8, wherein the method comprises administering 0.5 g/day.

Clause 11. Ribitol for use in the method of clause 8, wherein the method comprises administering 1.5 g/day.

Clause 12. Ribitol for use in the method of clause 8, wherein the method comprises administering 3 g/day.

Clause 13. Ribitol for use in the method of clause 8, wherein the method comprises administering 6 g/day.

Clause 14. Ribitol for use in the method of clause 8, wherein the method comprises administering 10 g/day.

Clause 15. Ribitol for use in the method of clause 8, wherein the method comprises administering 12 g/day.

Clause 16. Ribitol for use in the method of clause 8, wherein the method comprises administering 15 g/day.

Clause 17. Ribitol for use in the method of any one of clauses 1 to 16, comprising administering the dose of ribitol for at least one week, two weeks, or four weeks.

Clause 18. Ribitol for use in the method of any one of clauses 1 to 16, comprising administering the dose of ribitol for at least one month, two months, or four months.

Clause 19. Ribitol for use in the method of any one of clauses 1 to 16, comprising administering the dose of ribitol chronically.

Clause 20. Ribitol for use in the method of any one of clauses 1 to 19, wherein the disease or disorder is associated with a defect in Fukutin-related protein (FKRP).

Clause 21. Ribitol for use in the method of any one of clauses 1 to 20, wherein a mammal has a mutation in the gene encoding Fukutin-related protein (FKRP) that causes a partial or complete loss-of-function in FKRP.

Clause 22. Ribitol for use in the method of any one of clauses 1 to 21, wherein the disease or disorder is a muscular dystrophy.

Clause 23. Ribitol for use in the method of clause 22, wherein the muscular dystrophy is FKRP-related alpha-dystroglycanopathy.

Clause 24. Ribitol for use in the method of clause 23, wherein the disease or disorder is Limb-Girdle Muscular Dystrophy type 2i (LGMD2i).

Clause 25. Ribitol for use in the method of clause 22, wherein the muscular dystrophy is a fukutin (FKTN)-related alpha-dystroglycanopathy.

Clause 26. Ribitol for use in the method of clause 22, wherein the muscular dystrophy is a ISPD-related alpha-dystroglycanopathy.

Clause 27. Ribitol for use in the method of clause 25, wherein the FKTN-related alpha-dystroglycanopathy is Fukuyama Syndrome.

Clause 28. Ribitol for use in the method of any one of clauses 1 to 27, wherein the maximum observed concentration (Cₘₐₓ) of ribitol is between 125 and 2500 μg/mL.

Clause 29. Ribitol for use in the method of any one of clauses 1 to 28, wherein the area under the serum concentration-time curve (AUC0-24) for ribitol is between 350 (μg·h)/mL and 8000 (μg·h)/mL.

Clause 30. Ribitol for use in the method of any one of clauses 1 to 29, wherein the subject is a mammal.

Clause 31. Ribitol for use in the method of any one of clauses 1 to 29, wherein the subject is a human.

Clause 32. Ribitol for use in the method of any one of clauses 1 to 31, wherein the method restores and/or enhances functional glycosylation of α-DG.

Clause 33. Ribitol for use in the method of any one of clauses 1 to 31, wherein the method treats the disease or disorder.

Clause 34. A pharmaceutical composition, comprising ribitol and a pharmaceutically acceptable carrier or excipient.

Clause 35. The pharmaceutical compositions of clause 34, wherein the pharmaceutical composition is a solid, optionally a tablet or capsule.

Clause 36. The pharmaceutical compositions of clause 34, wherein the pharmaceutical composition is a solution.

Clause 37. The pharmaceutical composition of clause 36, wherein the carrier is water.

Clause 38. The pharmaceutical composition of clause 37, wherein the carrier is substantially pure water.

Clause 39. The pharmaceutical composition of clause 38, wherein the carrier is saline.

Clause 40. The pharmaceutical composition of any one of clauses 34 to 39, wherein the pharmaceutical composition comprises ribitol at between 0.2 g/mL and 10 g/mL.

Clause 41. A kit comprising the pharmaceutical composition of any one of clauses 34 to 40 and instructions for use in treating a disease or disorder.

Clause 42. A unit dose, comprising between 0.5 g and 15 g of ribitol.

### EXAMPLES

The following examples are put forth so as to provide those of ordinary skill in the art with a description of how the compositions and methods described herein may be used, made, and evaluated, and are intended to be purely illustrative of the invention and are not intended to limit the scope of what is regarding as the invention.

### Example 1: Nonclinical Testing

Ribitol is a pentose alcohol that is converted to CDP-ribitol (the substrate for Fukutin-related protein [FKRP]) in muscle. At high levels of ribitol, muscle with genetic defects in the FKRP enzyme can produce sufficiently high amounts of CDP-ribitol such that the biochemical defect caused by specific mutations in FKRP can be overcome, thereby enabling a restoration of the α-DG glycosylation levels which would be predicted to improve muscle performance.

The nonclinical pharmacology testing strategy for ribitol was designed to provide confidence in the hypothesis to increase matriglycan expression and muscle performance. Compromised matriglycan expression and muscle performance are hallmarks of Limb Girdle Muscular Dystrophy Type 2i (LGMD2i). Ribitol was tested in two models of LGMD2i FKRP mutant mice. In a model presenting a severe phenotype (P448L FKRP), ribitol demonstrated increased matriglycan expression and improved muscle performance. Histology showed decreases in muscular fibrosis and less regenerating fibers. Creatine kinase levels were improved in the presence of ribitol. A second murine model (L276I FKRP) presenting a milder form of LGMD2i also demonstrated increased matriglycan expression and improved treadmill running distance when treated with ribitol.

In the human ether-á-go-go-related gene (hERG) assay, the half maximal inhibitory concentration (IC₅₀) for ribitol was greater than 2.90 millimolar (mM). Cardiovascular and respiratory effects were evaluated in conscious male Bama minipigs and there was no ribitol-related effect on blood pressure, heart rate, electrocardiogram (ECG), or respiratory parameters up to the highest single dose evaluated (2 grams per kilogram (g/kg)). The effect of a single dose of ribitol on the central nervous system (CNS) was evaluated in a functional observation battery (FOB) in male CD-1 mice at dose levels up to 2 g/kg. There was no treatment-related effect on any of the assessed parameters at any timepoint. The no observed effect level (NOEL) for ribitol-related cardiovascular (CV) and respiratory effects in minipigs and CNS effects in mice was determined to be 2 g/kg the highest dose tested in each study, providing safety margins of 19 fold and 227-fold for the proposed clinical starting dose (0.5 g/day in a 60 kg patient). This dose is approximately 4-fold above the efficacious ribitol dose of 0.5 g/kg in FKRP mutant mouse models.

The pharmacokinetics (PK) of ribitol following single-dose administration was studied in CD-1 mice and Bama minipigs. There was no difference in PK of ribitol between male and female animals.

Following an oral administration, ribitol was rapidly absorbed with the maximum plasma concentration (Cₘₐₓ) observed (Tₘₐₓ) at less than 2 hours. The oral bioavailability was approximately 22.1% to 30.9% in the mouse and 55.9% to 70.2% in minipig. There was no apparent sex difference in systemic ribitol exposure (area under the curve from time zero to 24 hours post first dose [AUC₀₂₄] and Cₘₐₓ) following oral administration of up to 1.5 g/kg/dose twice daily ([BID]; 3.0 g/kg/day). The exposure of ribitol increased proportionally with dose following repeated dosing between 0.5 to 1.5 g/kg/dose BID (1.0 to 3.0/kg/day) in the mouse and 0.1 to 1 g/kg/dose BID (0.2 to 2 g/kg/day, respectively) in the minipig. There was no apparent drug accumulation for ribitol observed at any dose level after 28 days repeated oral administration.

Following an intravenous (IV) administration in the mouse, the exposure of ribitol increased proportionally with dose between 10 and 30 mg/kg/day, but less than dose proportionally between 30 and 100 mg/kg/day. In the minipig, the exposure of ribitol increased proportionally with dose between 10 and 100 mg/kg/day. Systemic clearance of ribitol was moderate in both mouse and minipig. Volume of distribution at steady state (Vₛₛ) was large in the mouse (1.49 to 6.74 L/kg), but moderate in the minipig (0.417 to 0.603 L/kg).

An in vitro permeability study in Caco-2 cells suggests that ribitol has low permeability and is not a P-glycoprotein (P-gp) substrate. Ribitol had low plasma protein binding at concentrations of 0.49 and 1.31 mM. Binding of ribitol to human plasma (34.6%-36.5%) was slightly lower than those in CD-1 mouse, Sprague-Dawley rat, and Gottingen minipig (38.6%-44.6%).

Ribitol was stable when incubated for up to 2 hours in liver microsomes, cytosol, and hepatocytes of CD-1 mice, Sprague-Dawley rats, Gottingen minipigs, or humans which suggests that liver metabolism is unlikely to be involved in the clearance of ribitol. Renal excretion studies of ribitol in animals are planned and will also be evaluated in healthy volunteers during the Phase 1 Single Ascending Dose (SAD) and Multiple Ascending Dose (MAD) studies.

Oral ribitol was well tolerated in maximum tolerated dose (MTD)/7-day dose range finding (DRF) non-Good Laboratory Practice (GLP) studies and in 28-day repeat-dose GLP toxicity studies with doses up to 1.5 g/dose BID (3 g/kg/day) in CD-1 mice and up to 1 g/kg/dose BID (2 g/kg/day) in Bama minipig, the highest doses evaluated. There was no treatment-related mortality or change in clinical pathology. These data are consistent with a pharmacology study that demonstrated no change in serum glucose levels in normal mice following a single oral dose of 10 g/kg/day of ribitol and no increase in serum triglyceride levels in FKRP P448L mice given oral doses of up to (10 g/kg) ribitol weekly from 7 weeks to one year. The only treatment related observation was soft or mild loose stools at the highest dose level in both species in the 7-day MTD studies. In the 28-day studies in mouse only, soft or abnormal stools were only observed at low incidence at low and moderate doses and resolved within 2 to 8 days without treatment discontinuation. Correlative changes in serum electrolytes or macroscopic observations in the gastrointestinal (GI) tract at doses up to 1.5 g/kg/dose BID (3 g/kg/day) in mice and 1 g/kg/dose BID (2 g/kg/day) in minipigs. There were no microscopic findings in the main study and recovery animals.

The proposed initial human doses of 0.5, 1.5, and 3 g/day are approximately 29- to 227-fold lower than the human equivalent dose (HED) determined from the mouse and minipig studies, respectively (assuming a 60 kg human weight and taking body surface area into consideration; Table 4). Additionally, the proposed human dose will be lower than the anticipated human therapeutic dose. Dosing up to and beyond the lowest no observed adverse effect level (NOAEL) exposure observed in the 28-day oral repeat-dose toxicity studies will be dependent on the available human safety data. Available data from toxicity studies in normal mice and minipigs demonstrate a favorable safety profile at doses up to 1.5 g/kg/dose BID (3 g/kg/day) in mice and 1 g/kg/dose BID (2 g/kg/day) in minipigs, the highest doses tested and support the proposed clinical trial.

Further preclinical proof of concept studies in a mouse model of LGMD2i consisting of the severe P448L FKRP mutation provided support for the proposed clinical therapeutic strategy as summarized in Example 2 below.

The proof-of-concept study confirmed that administration of exogenous ribitol orally or intravenously restored molecular, cellular, and functional phenotypes compared with untreated mice. Treated mice demonstrated up to 4 times greater levels of ribitol, ribitol-5-phosphate, and cytidine 5-diphosphate-ribitol (CDP-ribitol) in heart and leg muscles versus untreated mice. Glycosylation levels increased from undetectable levels to up to 26% in skeletal muscles with a reduction in disease specific pathology such as central nucleated fibers (diaphragm) and fibrosis (heart). Functional improvement was demonstrated in treadmill mobility testing and respiratory function.

TABLE 4
Estimated Safety Margins for ribitol Based on Nonclinical Data

| Species/ | | NOAEL^{a} | | Cₘₐₓ | AUC_{0-24 h} | HED^{b} | | MOS^{c} |
|---|---|---|---|---|---|---|---|---|
| Duration | ROA | g/kg/day | g/m³/day | μg/mL | μg*h/mL | g/kg/day | g/day^{d} | fold |
| Mouse/ | PO | 3.0 | 9.0 | 377 | 1340 | 0.2 | 14.6 | 29.2 |
| 28-Day | | | | (M) | (M) | | | |
| | | | | 294 | 839 | | | |
| | | | | (F) | (F) | | | |
| Minipig/ | PO | 2.0 | 70 | 475 | 2560 | 1.9 | 114 | 227 |
| 28-Day | | | | (M) | (M) | | | |
| | | | | 281 | 1980 | | | |
| | | | | (F) | (F) | | | |

AUC_{0-24 h} = area under the plasma concentration vs. time curve from time zero to 24 hours post-dose;
Cₘₐₓ = maximum plasma concentration;
F = female;
g = gram;
HED = human equivalent dose;
h = hour;
kg = kilogram;
M = male;
mL = milliliter;
MOS = margin of safety;
NOAEL = no-observed-adverse-effect-level;
PO = oral;
ROA = route of administration;
μg = microgram
^{a}Based upon study data interpretation and absence of any adverse toxicity.
^{b}Based on allometric conversion between species to achieve a HED. The allometric conversion factors used for mouse, minipig, and human are 3, 35, and 37, respectively.
^{c}Calculated by dividing the HED (g/day) study by the proposed human start dose of 0.5 g/day.
^{d}Calculated based on a 60 kg human.

### Example 2: Non-Clinical Pharmacology, Pharmacokinetics, and Toxicology

### Primary Pharmacology

The ribitol nonclinical pharmacology program is comprised of in vivo pharmacology and safety pharmacology studies.

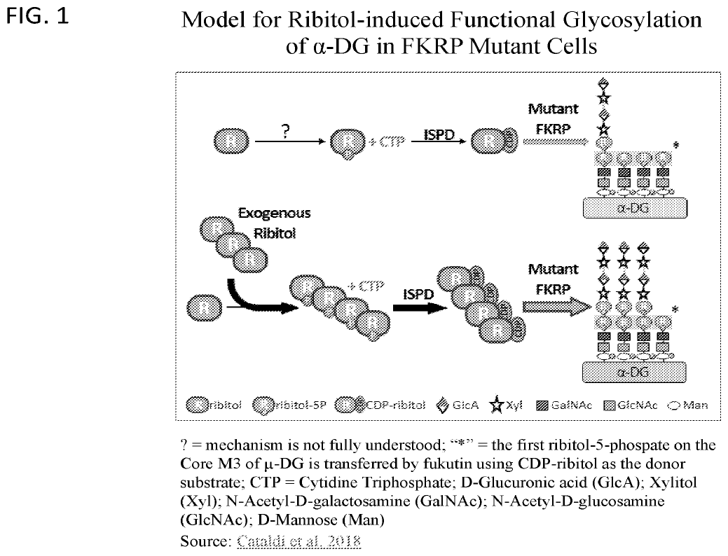

FKRP was recently identified as a ribitol-5-phosphate transferase that utilizes cytidine 5′diphosphate (CDP)-ribitol as the substrate for the extension of the laminin binding biglycan (matriglycan) on α-DG, a critical step for muscle integrity. An earlier study suggested that ribitol administration can lead to an increase of CDP-ribitol levels in cells. It has also been demonstrated that mutant FKRPs, even with a P448L mutation associated with Congenital Muscular Dystrophy (CMD), may retain sufficient function to produce matriglycan as demonstrated by adeno-associated virus mediated gene therapy with the mutant FKRP as the transgene (Tucker et al. 2018). Supplementation with ribitol increases CDP-ribitol levels, the FKRP substrate, which in turn enhances the efficiency of FKRP function, thus compensating for reduced mutant FKRP function and restoration of the expression of matriglycan. While the exact biochemical mechanism of these FKRP mutations is not currently known, it is likely affecting the Michaelis constant (K_{M}) of the enzymatic reaction as large amounts of ribitol lead to increased expression of the matriglycan. This mechanism was principally established in several earlier studies, especially in an in vivo preclinical model of disease (Cataldi et al. 2018, Gerin et al 2015, Kanagawa et al 2016) (FIG. 1).

### In Vivo

In vivo primary pharmacology studies were conducted to determine the effect of ribitol on muscle matriglycan expression and muscle performance in mice. Two murine models of mutant FKRP were evaluated: one model presenting the congenital muscular dystrophy type 1C P448L FKRP mutation and the other presenting the more common and less severe LGMD2i L276I FKRP mutation. Ribitol was also evaluated in wild type mice to understand if additional matriglycan expression was observed in non-diseased muscle.

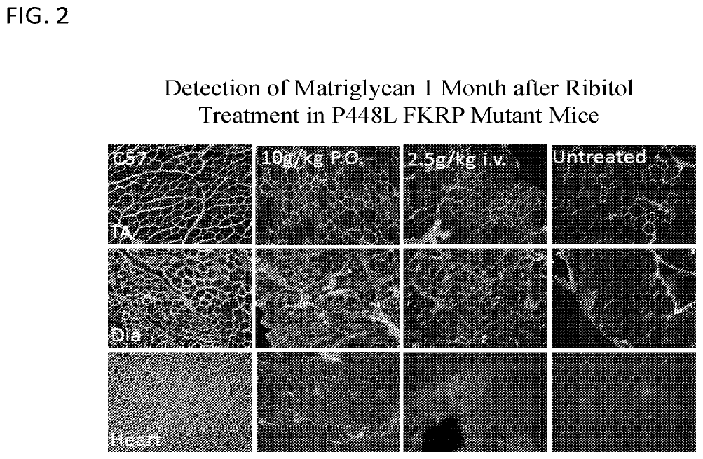

Ribitol was studied in a P448L FKRP mutant mouse model to assess drug-mediated improvements in matriglycan expression and muscle performance. In one study the mutant mice were treated with doses of 10 g/kg daily by oral gavage or 2.5 g/kg ribitol IV injection weekly for one month. Tissue immunohistochemistry showed that the matriglycan expression was improved with one month of ribitol relative to the vehicle control (FIG. 2).

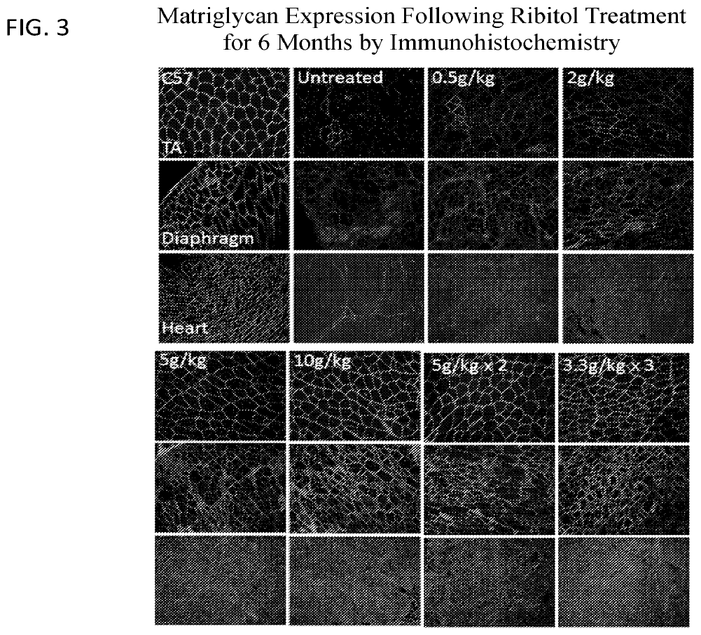

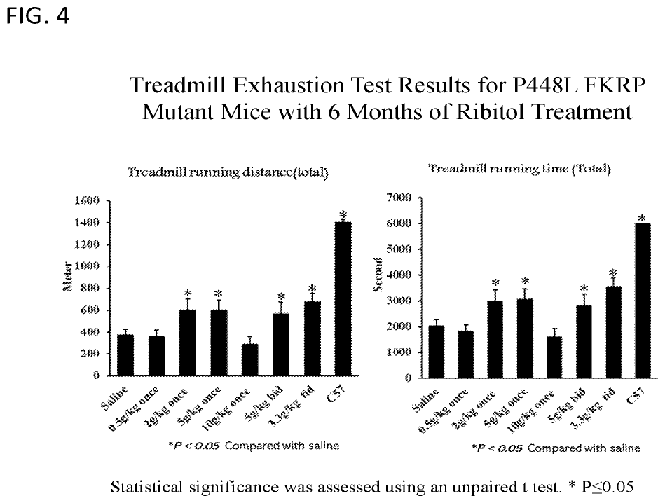

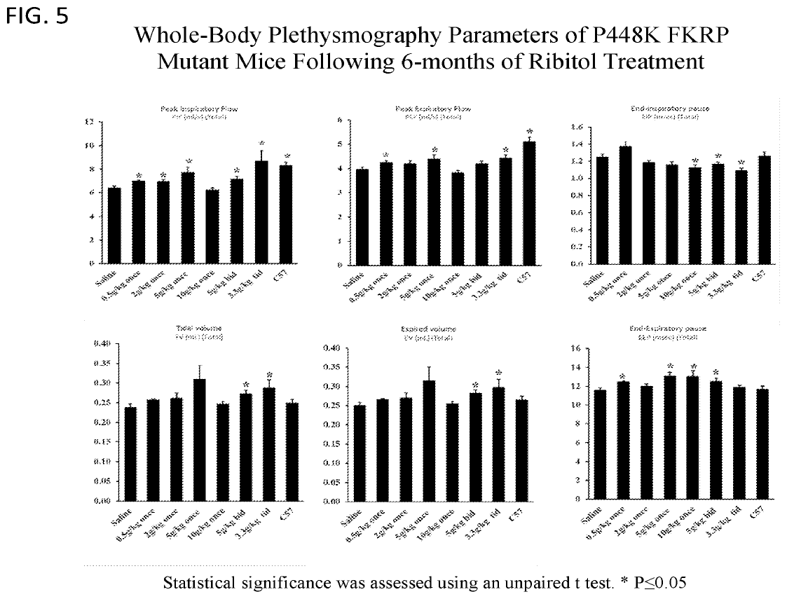

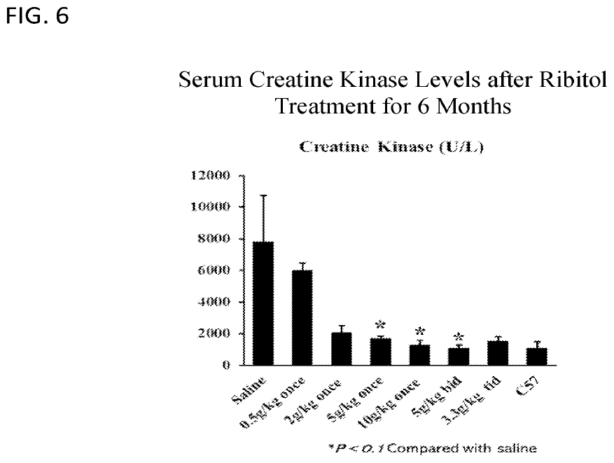

In a long-term efficacy study the FKRP P448L mutant mice were dosed up to one year by oral gavage. Matriglycan expression was increased as measured by immunohistochemistry and Western Blot analysis after six months of dosing at 2 g/kg, 5 g/kg, and 10 g/kg daily (FIG. 3). Treadmill exhaustion showed improvements in running distance and running time (FIGS. 4A-4B) at doses of 2 g/kg/day, 5 g/kg/day, 5 g/kg/dose BID (10 g/kg/day), and 3.3 g/kg/dose three times daily (TID) (9.9 g/kg/day). Diaphragmatic performance was assessed using whole body plethysmography (FIGS. 5A-5F). Improvements were seen in the end expiratory pause in the 0.5 g/kg, 5 g/kg, 10 g/kg, and 5 g/kg/dose BID (10 g/kg/day). Creatine kinase activity decreased with increasing doses of ribitol (FIG. 6) and histology showed decreases in fibrosis in the muscles.

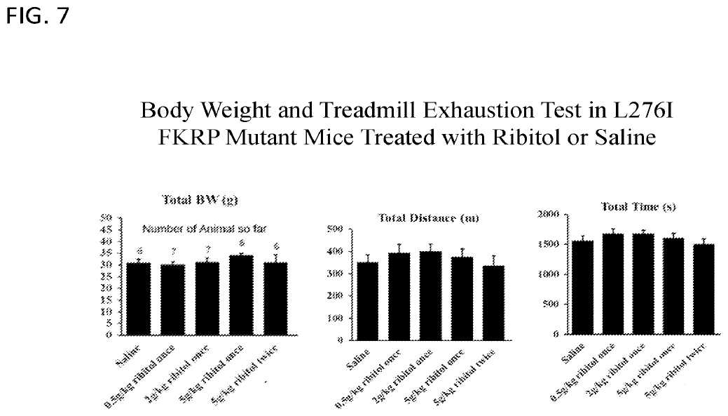

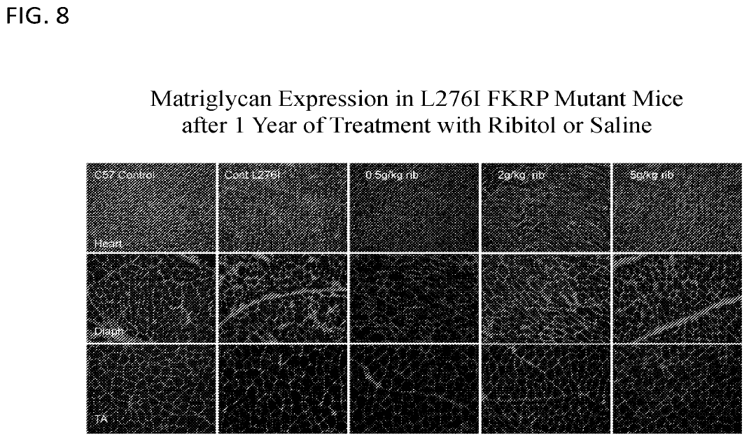

The more common LGMD2i FKRP L276I mutation was studied in a murine model of disease. After six months of dosing muscle performance was assessed using treadmill exhaustion tests, a trend toward improved running distance was seen in daily doses of 2 g/kg, 5 g/kg, 5 g/kg/dose BID (10 g/kg/day), and 3.3 g/kg/dose TID (9.9 g/kg/day) (FIG. 7B). Total running time was increased in these dosing groups (FIG. 7C). Immunohistochemistry showed increased matriglycan expression in all treatment groups (FIG. 8).

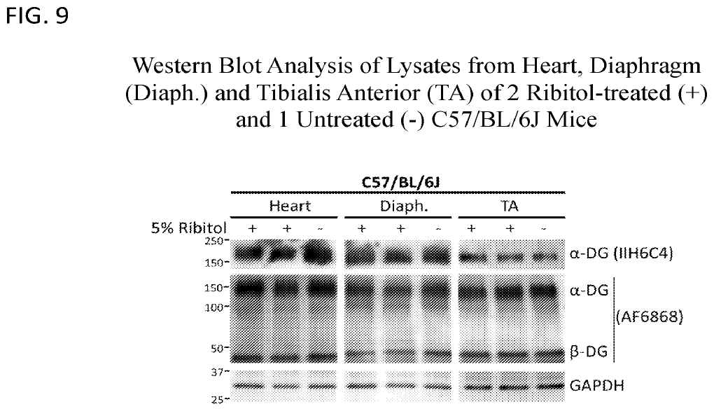

Wild type mice were dosed with ribitol to assess matriglycan expression in normal mice. The mice were treated for one month with 5% ribitol in drinking water ad libitum. Muscle samples were evaluated for matriglycan expression at the end of dosing via Western Blot analysis (FIG. 9). No increase in matriglycan expression was observed in the treatment groups as compared to the vehicle treated mice (FIG. 9).

### Safety Pharmacology

Effects of Ribitol on hERG Channel

The potential inhibitory effects of ribitol on electric current passing through hERG potassium channels (a surrogate for the rapidly activating, delayed rectifier cardiac potassium [I_{Kr}]current) stably expressed in a Chinese hamster ovary (CHO) cell line was evaluated using manual patch-clamp technique. Ribitol concentrations of up to 3 mM were used to evaluate the effects on hERG current. ribitol inhibited hERG current by 3.09% at 0.03 mM, 6.43% at 0.3 mM and 7.82% at 3 mM, respectively. Therefore, the concentrations chosen for the definitive hERG assay were 0.1, 0.3, 1, and 3 mM, the detected concentrations of postperfusion solution were 0.101, 0.293, 0.999, and 2.90 mM, respectively. The IC₅₀ for the inhibitory effect of ribitol was greater than 2.90 mM. CV and respiratory effects were evaluated in conscious male Bama minipigs and there was no ribitol related effect on blood pressure, heart rate, ECG, or respiratory parameters up to the highest dose evaluated (2.0 g/kg).

### Effect of Ribitol on Cardiovascular and Respiratory Parameters in Conscious Telemetered Minipigs

Minipigs received 0 (vehicle, purified water), 0.3, 1.0, or 2.0 g/kg of ribitol in a Latinsquare design on Days 1, 12, 17, and 25, respectively. The animals were observed for clinical signs and mortality daily. Body weights were measured at baseline and on each dosing day. On each dosing occasion, ECG waveforms, heart rate, and blood pressure data were recorded from at least 2 hours prior to dose (to establish baseline values) to approximately 24 hours post-dose. ECG tracings of a minimum of 30 seconds duration were obtained from all minipigs twice prior to each dose (at least 30 minutes apart) and at 0.5, 2, 4, 8, 12, and 24 hours post dose.

There was no ribitol-related change in blood pressure, heart rate, ECG, or respiratory parameters at all dose levels up to 2.0 g/kg. Thus, the NOEL was determined to be 2.0 g/kg, approximately 4-fold above the efficacious dose of 0.5 g/kg ribitol in FKRP mutant mouse models.

### Effect of Ribitol on the Mouse Functional Observational Battery (FOB)

In this GLP study, the potential effects of ribitol on the CNS were evaluated in CD-1 mice utilizing the FOB. Male mice were randomly assigned to 4 groups of 8 and were administered a single oral dose of 0 (vehicle, purified water), 0.5, 1.0, or 2.0 g/kg ribitol. The FOB tests were conducted pre dose, and at 0.5, 1, 2, 4, and 24 hours post dose. The timepoints were selected based on the Tₘₐₓ of ribitol in mice at approximately 1 hour. There were no treatment-related effects on any of the assessed parameters at any timepoint. Therefore, the NOEL was determined to be 2.0 g/kg ribitol, the highest dose tested. This dose is approximately 4-fold above the efficacious dose of 0.5 g/kg ribitol in FKRP mutant mouse models.

### Nonclinical Pharmacokinetics

### Absorption

### In Vitro Permeability Study

The absorption of ribitol was evaluated in Caco-2 cells at a concentration of 300 μg/mL. The apparent permeability values for apical (A)-to-basal (B) and B-to-A transport were <0.4 and <0.1 cm/sec, respectively. The data suggests that ribitol has low permeability and is not a P-glycoprotein (P-gp) substrate.

### Single Dose Administration

### Single Oral Administration in CD-1 Mouse and Bama Minipig

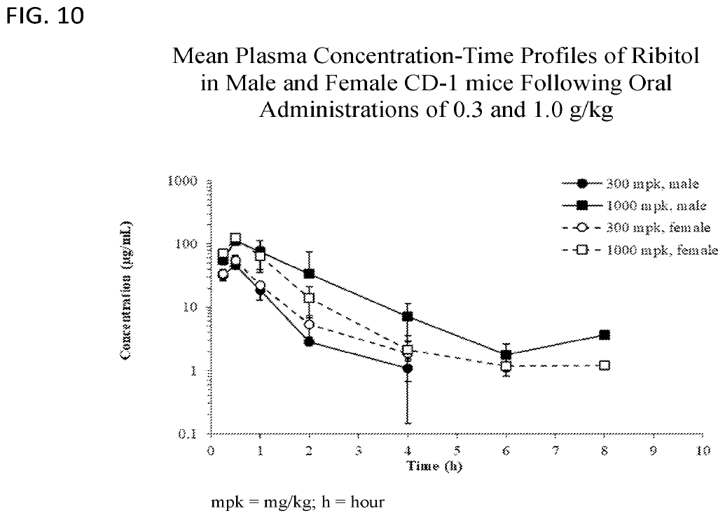

The PK of ribitol was studied in male and female CD-1 mice following two oral doses at 0.3 and 1.0 g/kg (FIG. 10). Blood samples were collected serially up to 24 hours post dose. Mean plasma concentration-time profiles are shown in FIG. 10. Pharmacokinetic parameters are summarized in Table 5.

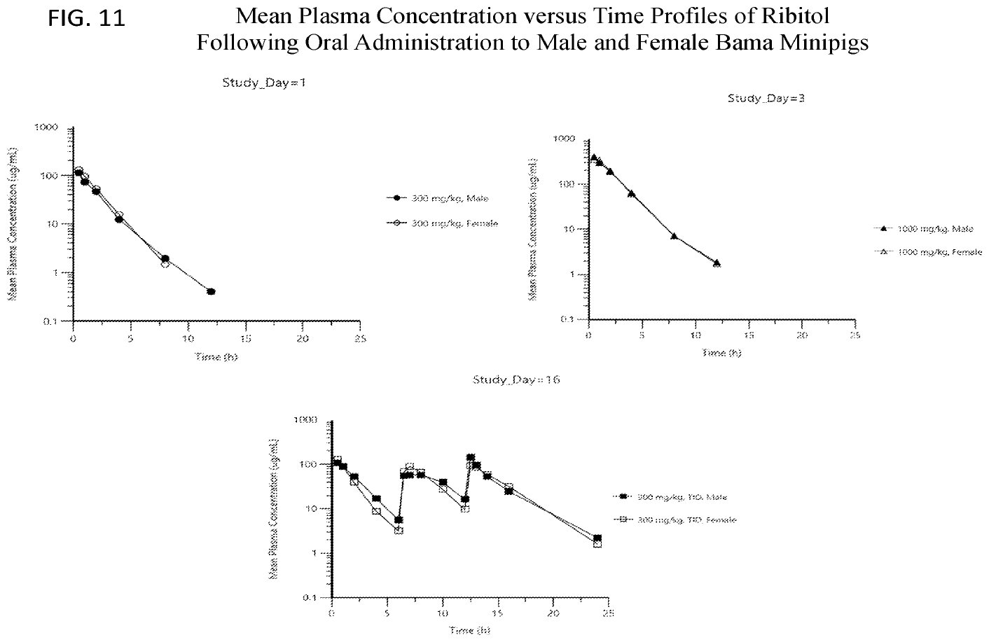

The PK of ribitol was studied in 3 male and 3 female Bama minipigs following escalating PO dose levels using the same animals with a 48 hour washout period at 0.3 g/kg (Day 1) (FIG. 11A), 1.0 g/kg (Day 3) (FIG. 11B) and 0.3 g/kg TID (Day 16) (FIG. 11C). On Days 1 and 3, blood samples were collected at 0, 0.5, 1, 2, 4, 8, 12, and 24 hours post dose from all animals. On Day 16, blood samples were collected from all animals at 0, 0.5, 1, 2, 4, 6 (pre-second dose), 6.5, 7, 8, 10, 12 (pre-third dose), 12.5, 13, 14, 16, and 24 hours post dose. Mean plasma concentration-time profiles are shown in FIGS. 11A-11C. PK parameters are summarized in Table 5.

In both species, ribitol was rapidly absorbed to reach Tₘₐₓ in less than 2 h. There was no apparent sex difference in the peak and systemic exposure (Cₘₐₓ and AUC₀₂₄) of ribitol. In both species, the exposure increased proportionally between 0.3 g/kg and 1.0 g/kg. The oral bioavailability was approximately 22.1% to 30.9% in the mouse and 55.9% to 70.2% in the minipig.

TABLE 5
Mean (SD) Pharmacokinetic Parameters of Ribitol Following
a Single Oral Dose in CD-1 Mouse and Bama Minipig

| | | Male | | | | | Female | | | | |
|---|---|---|---|---|---|---|---|---|---|---|---|
| | | | | Cₘₐₓ | AUC_{0-∞} | | | | Cₘₐₓ | AUC_{0-∞} | |
| | Dose | T_{1/2} | Tₘₐₓ | (μg/ | (μg · h/ | % F | T_{1/2} | Tₘₐₓ | (μg/ | (μg · h/ | % F |
| Species | (g/kg) | (h) | (h) | mL) | mL) | ^{a} | (h) | (h) | mL) | mL) | ^{a} |
| CD-1 Mouse | 0.3 | 0.677 | 0.500 | 45.6 | 41.6 | 24.6 | 1.31 | 0.500 | 55.0 | 55.9 | 30.9 |
| (n = 3/dose/ | | (0.105) | (0.00) | (2.64) | (2.06) | — | (1.20) | (0.00) | (10.1) | (6.64) | — |
| sex) | 1.0 | 1.31 | 0.500 | 112 | 173 | 30.5 | 1.17 | 0.500 | 124 | 129 | 22.1 |
| | | (0.381) | (0.00) | (18.2) | (106) | — | (0.394) | (0.00) | (7.09) | (15.8) | — |
| Bama | 0.3 | 1.60 | 0.500 | 112 | 210 | 55.9 | 1.23 | 0.670 | 128 | 245 | 65.3 |
| Minipig | | (0.361) | (0.00) | (39.6) | (59.7) | — | (0.153) | (0.289) | (26.0) | (26.1) | — |
| (n = 3/dose/ | 1.0 | 1.47 | 0.500 | 404 | 869 | 69.4 | 1.43 | 0.670 | 368 | 878 | 70.2 |
| sex) | | (0.153) | (0.00) | (65.5) | (33.5) | — | (0.252) | (0.289) | (44.7) | (35.4) | — |

AUC_{0-∞} = area under the plasma concentration versus time curve from time 0 extrapolated to infinity;
Cₘₐₓ = maximum plasma concentration;
% F = absolute oral bioavailability;
SD = standard deviation;
T_{1/2} = half-life;
Tₘₐₓ = time to maximum plasma concentration;
“—” = not evaluated
^{a} % F was calculated by dividing mean dose-normalized AUC_{0-inf} from oral administration by mean dose-normalized AUC_{0-inf} from IV of the corresponding sex. Three mean AUC_{0-inf} IV for male and female animals at 10, 30, and 100 mg/kg were used and the resulting % F was the average of these three values.

### PK Comparison Following Once, Twice, or Three Times Daily Oral Dosing in the CD-1 Mouse

PK of ribitol was evaluated following a once daily (QD), BID or TID dosing regimens of 0.3 mg/kg/dose with 6 hours between subsequent doses. Each regimen was studied in 3 male and 3 female CD-1 mice. PK of ribitol following the final dose was compared among these three regimens. The first and the second subsets of mice receiving QD and BID of ribitol were bled at 0.5, 1, 2, 4, and 6 hours post QD dosing and the second dose of the BID dosing, respectively; the third subset of mice receiving TID of ribitol were bled at 0.5, 1, 2, 4 and 12 hours post the third dose. Mean PK parameters were summarized in Table 6.

The PK parameters of the last dose of ribitol following QD, BID and TID administration were comparable between dose regimens and did not differ between genders. Comparing 0.3 g/kg single dose and 0.3 g/kg TID (0.9 g/kg/day) the AUC_{0∞} of ribitol increased proportionally with dose while Cₘₐₓ remained the same suggesting no drug accumulation.

TABLE 6
PK Parameters of Ribitol in Male and Female
CD-1 Mice Following Oral Administration at
0.3 g/kg with Different Regimens (mean ± SD)

| | | | 0.3 g/kg/dose | | 0.3 g/kg/dose | |
|---|---|---|---|---|---|---|
| | 0.3 g/kg QD | | BID | | TID | |
| | Male | Female | Male | Female | Male | Female |
| Parameters | (n = 3) | (n = 3) | (n = 3) | (n = 3) | (n = 3) | (n = 3) |
| Cₘₐₓ | 34.2 ± | 33.3 ± | 27.4 ± | 28.3 ± | 31.4 ± | 43.9 ± |
| (μg/mL) | 7.45 | 8.93 | 4.48 | 4.12 | 4.85 | 25.3 |
| Tₘₐₓ (h) | 0.50 ± | 0.50 ± | 0.50 ± | 0.50 ± | 0.50 ± | 0.50 ± |
| | 0.00 | 0.00 | 0.00 | 0.00 | 0.00 | 0.00 |
| T_{1/2} (h) | 0.839 ± | 1.17 ± | 1.50 ± | 0.984 ± | 0.454 ± | 0.530 ± |
| | 0.252 | 0.278 | 0.594 | 0.149 | 0.151 | 0.0892 |
| AUC_{0-∞} | 45.2 ± | 47.1 ± | 49.6 ± | 48.3 ± | 28.2 ± | 40.6 ± |
| (μg · h/mL) | 11.3 | 5.02 | 18.9 | 15.0 | 3.13 | 9.92 |

AUC_{0-∞} = area under the plasma concentration versus time curve from time 0 extrapolated to infinity;
BID = once daily;
Cₘₐₓ = maximum plasma concentration;
SD—standard deviation;
TID = three times daily;
Tₘₐₓ = time to maximum plasma concentration;
T_{1/2} = half-life;
QD = once daily

### Repeat Dose Administration in CD-1 Mouse and Bama Minipig

### Repeat Dose Administration in CD-1 Mouse

### 7-Day CD-1 Mouse Repeat-Dose Oral Toxicokinetics

Toxicokinetics were evaluated as part of a 7-day oral repeat-dose dose range finding study in CD-1 mice. Briefly, CD-1 mice were given 0, 0.5, 1.0, or 1.5 g/kg/dose BID (0, 1.0, 2.0, or 3.0 g/kg/day; 5 to 7 hours apart) ribitol by oral gavage. On Days 1 and 7, blood was collected for plasma toxicokinetics (TK) from the treatment groups at 0 (pre-first dose), 0.5, 2, 4, 6 (pre-second dose), 6.5, 8, 10, 12, and 24 hours post-first dose. In the vehicle control group, blood was collected for plasma TK on Day 1 and Day 7 at 0.5 and 6.5 hours post-first dose.

Plasma TK parameters are shown in Table 7. Maximum ribitol plasma concentrations were observed at 6.5 hours after the first dose on Days 1 and 7 (with exception of 0.5 hours for females receiving 0.5 g/kg BID on Day 1). There were no apparent sex differences in peak concentrations and systemic exposure (Cₘₐₓ and AUC₀₂₄). As the dose increased from 0.5 to 1.5 g/kg/dose BID (1.0 to 3.0 g/kg/day), the ribitol exposures (Cₘₐₓ and AUC₀₂₄) increased dose proportionally in males and females on Days 1 and 7. Ribitol exposures were similar on Days 1 and 7 indicating no apparent drug accumulation at any dose level.

TABLE 7
Ribitol Toxicokinetics after 7-Day Oral Repeat
Dose Administration in the CD-1 Mouse

| | Ribitol Dose (g/kg/dose BID) | | | | | |
|---|---|---|---|---|---|---|
| | 0.5 | | 1.0 | | 1.5 | |
| Parameter | M | F | M | F | M | F |

Day 1

| | | | | | | |
|---|---|---|---|---|---|---|
| Cₘₐₓ (μg/mL) | 121 | 141 | 265 | 282 | 316 | 339 |
| Tₘₐₓ (h) | 6.5 | 0.5 | 6.5 | 6.5 | 6.5 | 6.5 |
| AUC₀₋₂₄ (μg*h/mL) | 246 | 298 | 414 | 427 | 595 | 504 |

Day 7

| | | | | | | |
|---|---|---|---|---|---|---|
| Cₘₐₓ (μg/mL) | 118 | 100 | 237 | 210 | 305 | 266 |
| Tₘₐₓ (h) | 6.5 | 6.5 | 6.5 | 6.5 | 6.5 | 6.5 |
| AUC₀₋₂₄ (μg*h/mL) | 170 | 218 | 345 | 319 | 562 | 602 |

AUC0-24 = area under the plasma concentration versus time curve from time 0 to 24 hours post dose;
BID = twice daily;
Cₘₐₓ = maximum plasma concentration;
F = female;
M = male;
Tₘₐₓ = time to maximum plasma concentration.

### 28-Day CD-1 Mouse Repeat-Dose Oral Toxicokinetics

Toxicokinetics of ribitol was evaluated as part of the 28-day toxicology study in male and female CD-1 mice. The animals received ribitol at dose levels of 0 [vehicle, purified water], 0.5, 1.0, or 1.5 g/kg/dose BID (1.0, 2.0, or 3.0 g/kg/day) for 1 or 28 days via oral gavage. Toxicokinetic animals were 12/sex in the control group and 48/sex/group in the treated groups. Blood samples were collected on Day 1 and Day 28 at pre-first dose, 0.5, 2, 4, 8 (prior to second dose), 8.5, 10, and 24 h.

The Cₘₐₓ, Tₘₐₓ, and AUC₀₂₄ₕ values of ribitol following BID oral administration of ribitol at 0.5, 1.0, or 1.5 g/kg/dose BID (1.0, 2.0, or 3.0 g/kg/day), to male and female mice for 28 days are presented in Table 8.

TABLE 8
Toxicokinetics of Ribitol Following Twice
Daily Dosing for 28 Days in CD-1 Mice

| Dose | | | Cₘₐₓ | Tₘₐₓ | AUC₀₋₂₄ |
|---|---|---|---|---|---|
| (g/kg/dose BID) | Study Day | Sex | (μg/mL) | (h) | (h*μg/mL) |
| 0.5 | 1 | Male | 124 | 0.5 | 351 |
| | | Female | 157 | 8.5 | 322 |
| | | Mean | 141 | — | 337 |
| | 28 | Male | 138 | 0.5 | 450 |
| | | Female | 141 | 8.5 | 393 |
| | | Mean | 140 | — | 422 |
| 1.0 | 1 | Male | 286 | 8.5 | 559 |
| | | Female | 227 | 8.5 | 520 |
| | | Mean | 257 | — | 540 |
| | 28 | Male | 238 | 8.5 | 818 |
| | | Female | 252 | 0.5 | 794 |
| | | Mean | 245 | — | 806 |
| 1.5 | 1 | Male | 433 | 8.5 | 972 |
| | | Female | 334 | 8.5 | 1070 |
| | | Mean | 384 | — | 1020 |
| | 28 | Male | 377 | 8.5 | 1340 |
| | | Female | 294 | 8.5 | 839 |
| | | Mean | 336 | — | 1090 |

AUC₀₋₂₄ = area under the plasma concentration versus time curve from time 0 to 24 hours post dose;
BID = twice daily;
Cₘₐₓ = maximum plasma concentration;
Tₘₐₓ = time to maximum plasma concentration

After single (Day 1) or repeated (Day 28) oral administration of ribitol to male and female CD-1 mice, Tₘₐₓ values for ribitol were observed at 0.5 hours post first dose and 0.5 hours post second dose. No apparent sex difference in systemic exposure (Cₘₐₓ and AUC₀₋₂₄) to ribitol was observed at any dose level. As the dosage increased from 0.5 to 1.5 g/kg/dose BID (1.0 to 3.0 g/kg/day), the peak concentration and systemic exposure (Cₘₐₓ and AUC₀₋₂₄) increased dose-proportionally in males and females on Day 1 and Day 28. There was no apparent drug accumulation for ribitol observed at any dose level after 28 days repeated oral administration.

Ribitol was well tolerated and did not result in any mortalities after twice daily oral administration of ribitol at dose levels of 0.5, 1.0, or 1.5 g/kg/dose BID (1.0, 2.0, or 3.0 g/kg/day). Under the conditions of the study, the NOAEL was considered to be 1.5 g/kg/dose BID (3.0 g/kg/day), at which the AUC₀₋₂₄ and Cₘₐₓ on Day 28 were 1340 h*μg/mL and 377 μg/mL for males, and 839 h*μg/mL and 294 μg/mL for females, respectively.

### Repeat Dose Administration in Bama Minipig

### 7-Day Bama Minipig Repeat-Dose Oral Toxicokinetics

Toxicokinetics were evaluated as part of a 7-day oral repeat-dose dose range finding study in Bama minipigs. Briefly, Bama minipigs (2/sex/group) were given 0 (vehicle, purified water), 0.1, 0.3, or 1.0 g/kg/dose BID (0.2, 0.6, 2.0 g/kg/day; 6 hours apart) ribitol by oral gavage. On Days 1 and 7, blood was taken for plasma TK at 0 (pre first dose), 0.5, 1, 2, 4, 6 (pre-second dose), 6.5, 7, 8, 10, and 24 hours post-first dose.

The TK results are shown in Table 9. The Tₘₐₓ values were observed between 0.5 and 6.5 hours post first dose on Days 1 and 7. There were no apparent sex differences in systemic exposure (AUC₀₋₂₄ and Cₘₐₓ) to ribitol at any dose level. As the dosage increased from 0.1 to 1.0 g/kg/dose BID (0.2 to 2.0 g/kg/day), the systemic exposure (Cₘₐₓ and AUC₀₂₄) was approximately dose proportional. There was no apparent accumulation following 7 days of repeated oral ribitol administration at doses up to 1.0 g/kg/dose BID (2.0 g/kg/day).

TABLE 9
Ribitol Toxicokinetics after 7-Day Oral Repeat
Dose Administration in the Bama Minipig

| | Ribitol Dose (g/kg BID) | | | | | |
|---|---|---|---|---|---|---|
| | 0.1 | | 0.3 | | 1.0 | |
| Parameter | M | F | M | F | M | F |

Day 1

| | | | | | | |
|---|---|---|---|---|---|---|
| Cₘₐₓ (μg/mL) | 35.5 | 53.1 | 117 | 112 | 308 | 299 |
| Tₘₐₓ (h) | 3.5 | 3.8 | 6.5 | 0.8 | 3.8 | 1.3 |
| AUC₀₋₂₄ (μg*h/mL) | 160 | 284 | 504 | 676 | 169 | 1750 |

Day 7

| | | | | | | |
|---|---|---|---|---|---|---|
| Cₘₐₓ (μg/mL) | 45.1 | 40.2 | 58.3 | 159 | 283 | 331 |
| Tₘₐₓ (h) | 3.5 | 0.8 | 0.5 | 0.5 | 1.3 | 1.3 |
| AUC₀₋₂₄ (μg*h/mL) | 183 | 317 | 438 | 784 | 1920 | 1870 |

AUC₀₋₂₄ = area under the plasma concentration versus time curve from time 0 to 24 hours post dose;
BID = twice daily;
Cₘₐₓ = maximum plasma concentration;
F = female;
M = male;
Tₘₐₓ = time to maximum plasma concentration

### 28-Day Bama Minipig Repeat-Dose Oral Toxicokinetics

Toxicokinetics of ribitol was evaluated as part of the 28-day toxicology study in male and female Bama minipig. Animals in the test article treated groups were administered ribitol twice daily (6±0.5 hours apart) by oral gavage at 0.1, 0.3, or 1.0 g/kg/dose BID (0.2, 0.6, 2.0 g/kg/day) for 28 days. Animals in control group were dosed for 28 days twice daily with the vehicle only (Purified Water). On Days 1 and 28, blood samples were collected at 0 (pre first dose), 0.5, 1, 2, 4, 6 (pre-second dose), 6.5, 7, 8, 10, 12, and 24 hours from all available animals.

The mean±SD for Cₘₐₓ and AUC₀₂₄ values and median (range) for Tₘₐₓ values of ribitol following BID oral administration of ribitol at 0.1, 0.3, or 1.0 g/kg/dose BID (0.2, 0.6, 2.0 g/kg/day) to male and female minipigs for 28 days are presented in Table 10.

TABLE 10
Toxicokinetics of Ribitol Following Twice Daily
Oral Dosing for 28 Days in Bama Minipig

| Dose | | | | | |
|---|---|---|---|---|---|
| (g/kg/ | Study | | Cₘₐₓ | Tₘₐₓ | AUC₀₋₂₄ |
| dose BID) | Day | Sex | (ug/mL)^{a} | (h)^{a} | (h*μg/mL)^{a} |
| 0.1 | 1 | Male | 39.8 ± 16.8 | 6.8 (0.5-8.0) | 210 ± 75.0 |
| | | Female | 39.6 ± 23.3 | 5.0 (0.5-8.0) | 268 ± 129 |
| | 28 | Male | 37.2 ± 14.2 | 3.8 (0.5-7.0) | 215 ± 68.9 |
| | | Female | 37.2 ± 17.8 | 7.0 (1.0-8.0) | 257 ± 82.6 |
| 0.3 | 1 | Male | 104 ± 52.1 | 4.3 (0.5-7.0) | 539 ± 215 |
| | | Female | 98.4 ± 28 | 4.3 (1.0-7.0) | 605 ± 83.4 |
| | 28 | Male | 123 ± 78.2 | 0.8 (0.5-7.0) | 585 ± 227 |
| | | Female | 139 ± 40.9 | 4.3 (0.5-7.0) | 894 ± 125 |
| 1.0 | 1 | Male | 365 ± 93.6 | 3.8 (0.5-7.0) | 1930 ± 373 |
| | | Female | 261 ± 82.4 | 6.8 (1.0-7.0) | 1770 ± 582 |
| | 28 | Male | 475 ± 97.7 | 0.8 (0.5-7.0) | 2560 ± 526 |
| | | Female | 281 ± 141 | 7.0 (1.0-7.0) | 1980 ± 1040 |

AUC₀₋₂₄ = area under the plasma concentration versus time curve from time 0 to 24 hours post dose;
BID = twice daily;
Cₘₐₓ = maximum plasma concentration;
Tₘₐₓ = time to maximum plasma concentration
^{a}Data is presented as mean + standard deviation for Cₘₐₓ and AUC_{0-24 h} values, and median (range) for Tₘₐₓ values.

After single (Day 1) or repeated (Day 28) oral administration of ribitol to male and female minipigs, T values for ribitol were observed between 0.5 and 2.0 hours post after dose and between 0.5 and 2.0 hours post second dose. No marked sex difference in systemic exposure (C and AUC₀₋₂₄) to ribitol was observed at any dose level. As the dosage increased from 0.1 to 1 g/kg/dose BID (0.2 to 2.0 g/kg/day), the systemic exposure (Cₘₐₓ and AUC₀₋₂₄) to ribitol increased dose-proportionally in males and females on Days 1 and 28. There was no marked drug accumulation for ribitol observed at any dose level after a 28-day repeated oral administration.

The NOAEL for ribitol was considered to be 1 g/kg/dose BID (2 g/kg/day) in both sexes. Systemic exposure (Cₘₐₓ and AUC₀₋₂₄) at the NOAEL for the ribitol on Day 28 was 475 μg/mL and 2560 μg*h/mL, respectively, in males and 281 μg/mL and 1980 μg*h/mL, respectively, in females.

### Single Intravenous Administration in CD-1 Mouse and Bama Minipig

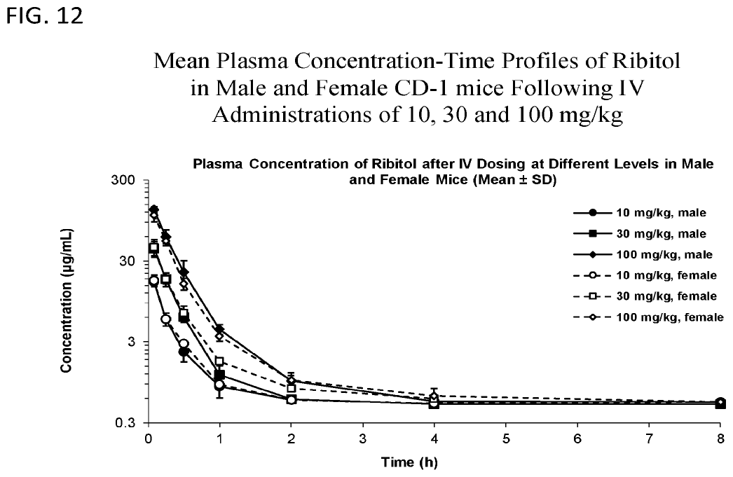

The PK of ribitol was studied in male and female CD-1 mice following three IV doses at 10, 30, and 100 mg/kg (n=3/dose/sex). Blood samples were collected serially up to 24 hours post dose. Mean plasma concentration-time profiles are shown in FIG. 12. Pharmacokinetic parameters were summarized in Table 11.

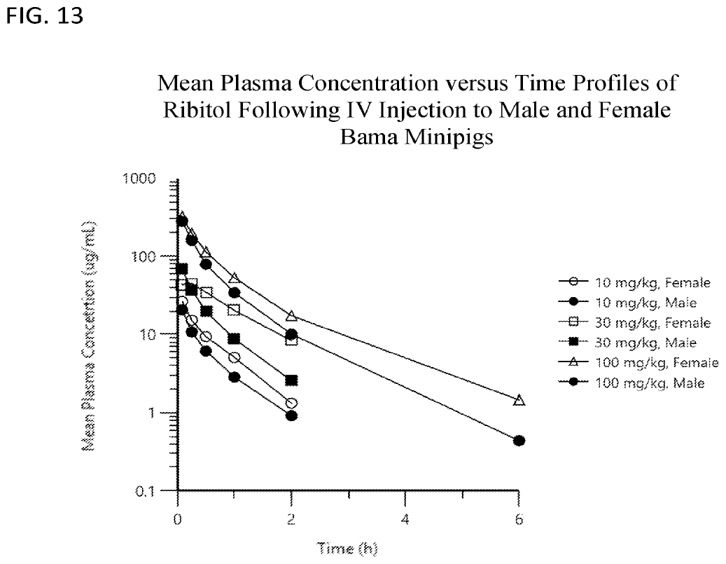

The PK of ribitol was studied in 2 male and 2 female Bama minipigs following escalating IV doses levels using the same animals with a 48 hour washout period at 10 mg/kg (Day 1), 30 mg/kg (Day 3) and 100 mg/kg (Day 5). On each dosing day, blood samples were collected serially up to 24 hours post dose. Mean plasma concentration-time profiles are shown in FIG. 13. PK parameters are summarized in Table 11.

In both species, there was no difference in PK of ribitol between male and female animals following IV administration. In the CD-1 mouse, the exposure of ribitol increased proportionally with dose between 10 and 30 mg/kg but less than dose-proportional between 30 and 100 mg/kg. In the Bama minipig, the exposure of ribitol increased proportionally with dose between 10 and 100 mg/kg.

Systemic clearance (CLₛ) of ribitol was moderate in both mouse and minipig. The CLₛ in the mouse was 12.6 to 25.0 mL/min/kg at 10 and 30 mg/kg but was higher at 100 mg/kg (33.6-38.9 mL/min/kg). In the minipig, the CLs was similar across the dose range of 10-100 mg/kg, ranging from 7.95 to 15.1 mL/min/kg.

Volume of distribution at steady state was large in the mouse (1.49 to 6.74 L/kg) but moderate in the minipig (0.417 to 0.603 L/kg).

Ribitol had a short half-life (T_{1/2}) in both mouse and minipig. The T_{1/2} following the IV dose in the mouse could not be accurately calculated due to the limited data point in the terminal phase. The T_{1/2} following the oral dose in the mouse was 0.677 to 1.31 hours (Table 5). The T_{1/2} following the IV dose in the minipig was 0.519 to 0.993 hour.

TABLE 11
Mean (SD) Pharmacokinetic Parameters of Ribitol Following
a Single Intravenous Dose in CD-1 Mouse and Bama Minipig

| | | Male | | | | Female | | | |
|---|---|---|---|---|---|---|---|---|---|
| | Dose | T_{1/2}^{a} | AUC_{0-∞} | Vₛₛ | CLₛ | T_{1/2}^{a} | AUC_{0-∞} | Vₛₛ | CLₛ |
| Species | (mg/kg) | (h) | (μg · h/mL) | (L/kg) | (mL/min/kg) | (h) | (μg · h/mL) | (L/kg) | (mL/min/kg) |
| CD-1 | 10 | — | 7.32 | 1.49 | 23.4 | — | 13.3 | 4.27 | 12.6 |
| Mouse | | | (1.51) | (0.770) | (4.31) | | (0.719) | (0.688) | (0.700) |
| (n = | 30 | — | 22.3 | 6.74 | 23.4 | — | 20.1 | 3.00 | 25.0 |
| 3/dose/ | | | (5.23) | (5.15) | (6.32) | | (1.87) | (2.11) | (2.34) |
| sex) | 100 | — | 49.7 | 2.01 | 33.6 | — | 43.1 | 2.67 | 38.9 |
| | | | (2.83) | (0.998 | (1.98) | | (4.09) | (1.28) | (3.53) |
| Bama | 10 | 0.545 | 11.2 | 0.603 | 15.1 | 0.556 | 17.0 | 0.417 | 10.1 |
| Minipig | 30 | 0.519 | 35.8 | 0.500 | 14.0 | 0.748 | 57.0 | 0.582 | 8.80 |
| (n = | 100 | 0.773 | 150 | 0.449 | 11.2 | 0.993 | 210 | 0.434 | 7.95 |
| 2/dose/ | | | | | | | | | |
| sex) | | | | | | | | | |

AUC_{0-∞} = area under the concentration versus time curve from time zero extrapolated to infinity;
CLₛ = systemic clearance;
SD = standard deviation;
T_{1/2} = half-life;
Vₛₛ = volume of distribution at steady state;
“—” = not evaluated
^{b} T_{1/2} in the CD-1 mouse could not be accurately estimated due to the limited number of timepoints in the terminal phase.

### Distribution

In vivo PK studies suggested that the Vₛₛ of ribitol was large in the CD-1 mouse (1.49 to 6.74 L/kg), but moderate in the Bama minipig (0.417 to 0.603 L/kg). Ribitol had low plasma protein binding in CD-1 mouse, Sprague-Dawley rat, Gottingen minipig and human at concentrations between 0.49 and 1.31 mM.

### Protein Binding

Protein binding studies with ribitol were performed in vitro using plasma from CD-1 mouse, Sprague-Dawley rat, Gottingen minipig, and human at 75 μg/mL (0.49 mM) and 200 μg/mL (1.31 mM) using the rapid equilibrium dialysis method. The percent of ribitol bound to plasma was approximately similar between the two concentrations tested in all species. Protein binding of ribitol was slightly lower in human plasma (34.6%-36.5%) than in the animals (38.6%-44.6%) (Table 12).

TABLE 12
In Vitro Plasma Protein Binding of Ribitol

| | % Binding | | | |
|---|---|---|---|---|
| | | Sprague- | | |
| | CD-1 | Dawley | Gottingen | |
| Concentration | Mouse | Rat | Minipig | Human |
| 75 μg/mL (0.49 mM) | 44.6 ± 1.3 | 43.3 ± 3.4 | 44.1 ± 1.0 | 36.5 ± 1.8 |
| 200 μg/mL (1.31 mM) | 42.5 ± 0.3 | 40.3 ± 0.5 | 38.6 ± 2.4 | 34.6 ± 0.3 |

### Metabolism

Metabolic stability of ribitol was conducted in liver microsomes, cytosol, and primary hepatocytes, from CD-1 mouse, Sprague-Dawley rat, Gottingen minipig, and human at a concentration of ribitol of 10 μM.

The results of metabolic stability including T_{1/2} (min) and intrinsic clearance values are summarized in Table 13 The data indicate that ribitol was not metabolized by the enzymes in the liver. Therefore, metabolism is unlikely to be a major elimination pathway of ribitol.

TABLE 13
Metabolic Stability of Ribitol in Liver
Microsomes, Cytosol, and Hepatocytes

| | Microsomes | | Cytosol | | Hepatocytes | |
|---|---|---|---|---|---|---|
| | | CL_{int, liver} | | CL_{int, liver} | | CL_{int, liver} |
| | T_{1/2} | (mL/ | T_{1/2} | (mL/ | T_{1/2} | (mL/ |
| Species | (min) | min/kg) | (min) | min/kg) | (min) | min/kg) |
| CD-1 Mouse | >187 | <29.3 | 578 | 4.75 | >364 | <22.6 |
| Sprague- | >187 | <13.3 | 258 | 4.83 | >364 | <8.89 |
| Dawley Rat | | | | | | |
| Gottingen | >187 | <5.56 | 196 | 2.71 | >364 | <9.16 |
| Minipig | | | | | | |
| Human | >187 | <6.66 | 381 | 1.64 | >364 | <5.28 |

CL_{int, liver} = apparent intrinsic liver clearance;
T_{1/2} = half-life

### Excretion

Based on the in vitro metabolic stability study, ribitol is unlikely to be eliminated by liver metabolism pathways. Studies to investigate renal excretion of ribitol in animals is being planned. Renal excretion of ribitol in human will also be evaluated in Phase I SAD and MAD studies.

### Pharmacokinetic Drug Interactions

In vitro studies to evaluate potential drug-drug interaction of ribitol are ongoing or planned for the CYP inhibition and induction by ribitol and the potential of ribitol to be a substrate or inhibitor of drug transporters.

### Summary

The PK of ribitol following single-dose administration was studied in CD-1 mouse and Bama minipig. There was no difference in PK of ribitol between male and female animals. Following an IV administration, in the mouse, the exposure of ribitol increased proportionally with dose between 10 and 30 mg/kg, but was less than dose-proportional between 30 and 100 mg/kg. In the minipig, the exposure of ribitol increased proportionally with dose between 10 and 100 mg/kg. Systemic clearance of ribitol was moderate in both mouse and minipig. Volume of distribution at steady state was large in the mouse (1.49 to 6.74 L/kg) but moderate in the minipig (0.417 to 0.603 L/kg).

Following an oral administration, ribitol was rapidly absorbed with Tₘₐₓ less than 2 h. Oral bioavailability was approximately 22.1% to 30.9% in the mouse and 55.9% to 70.2% in minipig. There was no apparent sex difference in peak concentrations and systemic exposure (Cₘₐₓ and AUC₀₂₄) of ribitol following oral administration up to 1.5 g/kg/dose BID (3.0 g/kg/day). The exposure of ribitol increased proportionally with dose following repeated twice daily dosing between 0.5 to 1.5 g/kg/dose BID (1.0 to 3.0 g/kg/day) in the mouse and 0.1 to 1 g/kg/dose BID (0.2 to 2.0 g/kg/day) in the minipig. There was no apparent ribitol drug accumulation observed at any dose level after 28 days repeated oral administration.

An in vitro permeability study in Caco-2 cells suggests that ribitol has low permeability and is not a P-gp substrate. Ribitol had low plasma protein binding. Binding of ribitol to human plasma (34.6%-36.5%) was slightly lower than those in CD-1 mouse, SpragueDawley rat and Gottingen minipig (38.6%-44.6%).

Ribitol was stable when incubated in liver microsomes, cytosol, and hepatocytes of CD-1 mouse, Sprague-Dawley rat, Gottingen minipig, or human, which suggests that metabolism is unlikely to be involved in the clearance of ribitol. Renal excretion studies of ribitol in animals are planned concurrent with the Phase 1 SAD/MAD study in healthy volunteers.

### Nonclinical Toxicology

ML Bio Solutions has conducted IND-enabling toxicology studies in mice and minipigs. These rodent and nonrodent species are considered pharmacologically relevant in that they have the same biochemical pathways for production and maintenance of glycosylated α-DG levels in muscle tissue. Additionally, ribitol is effective in a mouse model of the disease (P448L FKRP).

The route of administration in the toxicology studies is the same as that intended for the clinical program. The first in human study will be a Phase 1 SAD/MAD study in healthy volunteers in a Phase I unit. Ribitol will be administered QD or BID as an oral solution. Accordingly, animal toxicology studies were conducted with BID dosing.

The available data are considered adequate to support the first in human study. A brief description of these studies and available results are presented below.

### Toxicology Studies in Mice

### Maximum Tolerated Dose and the 7-Day Dose Range Finding Study

The purpose of this study was to determine the MTD following a single oral dose (Phase 1) and to characterize the toxicity and toxicokinetic profile following 7 days BID dosing with ribitol (Phase 2).

In Phase 1, Swiss Crl: CD1® mice (5/sex/group) were given ribitol at 0.3, 1.0, 1.5, or 2.0 g/kg/dose BID (0.6, 2.0, 3.0, or 4.0 g/kg/day) with 5 to 7 hours between doses. Following dosing, mice were observed for a 14-day post-dose period. Mice were assessed for viability, clinical signs, body weight, food consumption, and macroscopic findings at necropsy. There was no treatment-related mortality, change in clinical signs, body weight or food consumption or macroscopic findings in mice following single oral ribitol doses of up to 2.0 g/kg/dose BID (4.0 g/kg/day).

Doses were selected for Phase 2 based on the Phase 1 study results and the intended highest clinical dose of 12.0 g/day. In Phase 2, mice (5/sex/group) were given oral doses of 0 (vehicle control, purified water), 0.5, 1.0, or 1.5 g/kg/dose ribitol BID (1.0, 2.0, 3.0 g/kg/day) with 6 hours between doses. Mice were assessed for viability, clinical signs, body weight, food consumption, clinical pathology (hematology and serum chemistry), organ weights (adrenal glands, brain, heart, kidneys, liver with gall bladder, ovaries, spleen, testes, and thymus), and macroscopic pathology at necropsy. Blood was taken from a satellite group of animals (3/sex/timepoint) for plasma TK analysis on Days 1 and 7.

There was no treatment-related mortality or change in body weight or food consumption. At the 1.5 g/kg/dose BID dose (3.0 g/kg/day), abnormal soft stools were noted in 4 of 5 males from Days 1 to 7 and 1 of 5 females on Day 1. This was considered treatment-related and non-adverse due to the slight magnitude and incidence. There was no change in serum glucose levels, serum electrolytes, organ weights, or treatment-related macroscopic findings following oral doses of up to 1.5 g/kg/dose BID for 7 days (3.0 g/kg/day). The NOAEL for this study was considered 1.5 g/kg/dose BID (3.0 g/kg/day). The corresponding Day 7 Cₘₐₓ was 305 μg/mL for males and 266 μg/mL for females and AUC₀₋₂₄ was 562 μg*h/mL for males and 602 μg*h/mL for females.

28-Day Oral GLP Toxicity Study in Mice with 14-Day Recovery and TK

The purpose of this GLP study was to assess the potential toxicity, reversibility, persistence, or delayed effects of ribitol in mice following 28 days of BID oral gavage dosing. In addition, 20 mice/sex/group were treated for 28 days with either vehicle (purified water), 0.5, 1.0, or 1.5 g/kg/dose ribitol BID (1.0, 2.0, or 3.0 g/kg/day) followed by 14-day treatment free recovery period. In-life assessments included mortality, clinical signs including detailed clinical observations pre-test and once weekly during the dosing and recovery phase, body weight, food consumption, ophthalmology, and clinical pathology (hematology and serum chemistry). Terminal endpoints included macroscopic pathology, organ weights and histopathology. Blood was taken from a satellite group of mice for plasma TK (3/sex/timepoint) on Days 1 and 28.

All animals survived until scheduled termination. Ribitol was well tolerated, though soft or abnormal stools were observed at a low incidence in the 0.5 g/kg/dose BID group and in the 1 g/kg/dose BID group in 1 out of 20 males after 6 days of dosing. The soft or abnormal stools resolved within 2 to 8 days without treatment discontinuation. There was no test article-related effect on body weight, food consumption, ophthalmic examinations, hematology and serum chemistry examinations (including triglycerides, glucose, and electrolytes), gross observations, organ weights, or histopathology.

In summary, ribitol was well tolerated and did not result in any mortalities after BID oral administration of ribitol at dose levels of 0.5, 1.0 or 1.5 g/kg/dose BID (1.0, 2.0, or 3.0 g/kg/day) to CD-1 mice. Under the conditions of the study, the NOAEL was considered to be 1.5 g/kg/dose BID (3.0 g/kg/day), at which the AUC₀₋₂₄ and Cₘₐₓ on Day 28 were 1340 h*μg/mL and 377 μg/mL for males, and 839 h*μg/mL and 294 μg/mL for females, respectively.

### Toxicology Studies in the Minipig

### Maximum Tolerated Dose and 7-Day Dose Range Finding Study

The purpose of this study was to determine the MTD following two oral doses 6±0.5 hours apart (Phase 1) and to characterize the toxicity and TK profile following 7 days BID dosing with ribitol with 6±0.5 hours between doses in minipigs.

Minipigs were assessed for viability, clinical signs, body weight, food consumption, clinical pathology (hematology, coagulation, and serum chemistry), organ weights (adrenal glands, brain, heart, kidneys, liver with gall bladder, ovary, spleen, testes, and thymus), and macroscopic pathology at necropsy. Blood was taken for plasma TK analysis on Days 1 and 7.

In Phase 1, Bama minipigs (2/sex/group) were ribitol at 0.3, 1.0, 1.5, or 2.0 g/kg/dose BID (0.6, 2.0, 3.0, or 4.0 g/kg/day) with 5.5 to 6.5 hours between doses. Following dosing, minipigs were observed for a 14-day period. Minipigs were assessed for viability, clinical signs, body weight, food consumption, and macroscopic findings at necropsy.

Doses were selected for Phase 2 based on the Phase 1 study results and in compliance with the guidance criteria for high dose selection for general toxicity studies (ICH M3 [R2]). In this phase, minipigs (2/sex/group) were given oral doses of 0 (vehicle control, water), 0.1, 0.3, or 1.0 g/kg/dose ribitol BID with 5.5 to 6.5 hours between doses (0.2, 0.6, or 2.0 g/kg/day, respectively).

In Phase 1, all animals survived until scheduled necropsy. Only abnormal stool was noted at 2.0 g/kg/dose BID (4.0 g/kg/day) and resolved within the observation period. There was no treatment-related effect on body weight or food consumption or macroscopic observations. Under the conditions of this study, the MTD was considered ≥2.0 g/kg/dose BID (4.0 g/kg/day).

In Phase 2, there was no treatment-related mortality. Abnormal stools were noted at 1.0 g/kg BID in males during the first 5 days, and not observed after Day 5. There was no treatment-related change in body weight, food consumption, or clinical pathology (hematology, clinical chemistry, and coagulation) including serum triglycerides, glucose, and electrolytes. Following scheduled termination, there was no organ weight change and no macroscopic finding at necropsy, including the GI tract. Under the conditions of this study, ribitol was well tolerated when administered orally to minipigs at ≤1.0 g/kg/dose BID (2.0 g/kg/day). The oral 1.0 g/kg BID Day 7 Cₘₐₓ was 283 μg/mL for males and 331 μg/mL for females and AUC₀₋₂₄ was 1,920 μg*h/mL for males and 1,870 μg*h/mL for females.

28-Day Oral GLP Toxicity Study in Minipigs with 14-Day Recovery and Toxicokinetics

The purpose of this GLP study was to assess the potential toxicity, reversibility, persistence, or delayed effects of ribitol in minipigs following 28 days of BID oral gavage dosing with a 14 day recovery period. In addition, the plasma TK profile of ribitol was evaluated on Days 1 and 28. Four Bama minipigs/sex/group were administered 0 (vehicle control, purified water), 0.1, 0.3, or 1.0 g/kg/dose ribitol by oral gavage BID (0.2, 0.6, 2.0 g/kg/day; 6 hours apart) for 28 days. In addition, 2 minipigs/sex/group were treated for 28 days with either vehicle (water) or 1.0 g/kg/dose ribitol BID (2.0 g/kg/day) followed by 14-day treatment-free recovery period. In-life assessments included mortality, clinical signs including detailed clinical observations pre-test and once weekly during the dosing and recovery phase, body weight, food consumption, ophthalmology, electrocardiography, and clinical pathology (hematology, coagulation, serum chemistry, and urinalysis). Terminal endpoints included macroscopic pathology, organ weights and histopathology. Blood was taken for plasma TK on Days 1 and 28.

There was no ribitol-related change in clinical signs. Body weights and food consumption were well maintained during the study and there was no ribitol-related finding in ophthalmic examinations, electrocardiography, or clinical pathology. There was no ribitol-related organ weight change, macroscopic finding, or microscopic finding in the dosing or recovery phases.

The NOAEL for ribitol was considered to be 1.0 g/kg/dose BID (2.0 g/kg/day) in both sexes. Systemic exposure (Cₘₐₓ and AUC₀₋₂₄ₕ) at the NOAEL for the ribitol on Day 28 was 475 μg/mL and 2560 μg*h/mL, respectively, in males and 281 μg/mL and 1980 μg*h/mL, respectively, in females.

### In Vitro Micronucleus Assay

In this GLP-compliant study, ribitol was tested to evaluate the potential to induce micronuclei in HPBLs in both the absence and presence of S9. HPBL cells were treated for 4 hours in the absence and presence of S9, and for 24 hours in the absence of S9. Purified water was used as the vehicle.

In the preliminary toxicity assay, the doses tested ranged from 0.0152 to 152 μg/mL (1 mM), which was the limit dose for this assay. Cytotoxicity was not observed at any dose in any of the three treatment groups. Based upon these results, the doses chosen for the micronucleus assay ranged from 19 to 152 μg/mL for all three exposure groups.

Neither statistically significant nor dose dependent increases in micronuclei induction were observed at any dose in treatment groups with or without S9. These results indicate ribitol was negative for the induction of micronuclei in the presence and absence of the exogenous metabolic activation system.

### In Vivo Micronucleus Study

The GLP-compliant in vivo micronucleus assay in mice is ongoing and the data will be submitted to the IND prior to initiation of the Phase 1 MAD study.

### Discussion and Conclusions

ML Bio Solutions has conducted a comprehensive IND-enabling toxicology program for ribitol including 28-day repeat dose general toxicity studies in mice and minipigs and in vitro genetic toxicity studies to support the proposed Phase 1 trial.

Oral ribitol was well tolerated in MTD/7-day DRF non-GLP studies and in 28-day repeat dose GLP toxicity studies with doses up to 1.5 g/kg/dose BID (3.0 g/kg/day) in mice and up to 1.0 g/kg/dose BID (2.0 g/kg/day) in minipigs, the highest doses evaluated. There were no treatment-related mortalities, or changes in clinical pathology. Consistent with results in mice given 10 g/kg/day of ribitol, there were no changes in serum triglycerides or glucose in mice or minipigs. The only treatment-related observation was soft or mild loose stools at the highest dose level in both species in the 7-day MTD studies. In the 28-day studies in mouse only, soft or abnormal stools were only observed at low incidence, at moderate doses, and resolved within 2 to 8 days without treatment discontinuation. Although there were some mild loose or abnormal stools observed in the 7- and 28 day oral toxicity studies, there were no treatment-related changes in serum electrolytes or macroscopic observations at necropsy including the GI tract at doses up to 1.5 g/kg/dose BID (3.0 g/kg/day) in mice and 1.0 g/kg/dose BID (2.0 g/kg/day) in minipigs. It is not known whether these effects will be seen with more chronic dosing. There were no microscopic findings in the main study and recovery animals. Thus, there were no adverse findings at doses up to 1.5 g/kg/dose BID (3.0 g/kg/day) in mice and 1.0 g/kg/dose BID (2.0 g/kg/day) in minipigs, the highest doses tested.

The genotoxic potential of ribitol was evaluated in an in vitro Ames assay, and a micronucleus assay in HPBLs. Ribitol was devoid of genotoxicity potential in both in vitro assays. An in vivo micronucleus assay in mice is on-going and will be submitted to the IND prior the initial of the MAD phase of the clinical trial.

The initial human dose is proposed to be 0.5 g/day is approximately 29 to 227-fold lower than the human equivalent dose (HED) determined from the mouse and minipig studies, respectively (assuming a 60 kg human weight and taking body surface area into consideration; Table 14). Additionally, the proposed human dose will be lower than the anticipated human therapeutic dose. Dosing up to and beyond the lowest NOAEL exposure observed in the 28-day oral repeat-dose toxicity studies will be dependent on the available human safety data.

TABLE 14
Estimated Safety Margins for Ribitol Based on Nonclinical Data

| Species/ | | NOAEL^{a} | | Cₘₐₓ | | AUC_{0-24 h} | | HED^{b} | | MOS^{c} |
|---|---|---|---|---|---|---|---|---|---|---|
| Duration | ROA | g/kg/day | g/m³/day | μg/mL | | μg*h/mL | | g/kg/day | g/day^{d} | fold |
| Mouse/ | PO | 3.0 | 9.0 | 377 | (M) | 1340 | (F) | 0.2 | 14.6 | 29.2 |
| 28-Day | | | | 294 | (F) | 839 | (F) | | | |
| Minipig/ | PO | 2.0 | 70 | 475 | (M) | 2560 | (M) | 1.9 | 114 | 227 |
| 28-Day | | | | 281 | (F) | 1980 | (F) | | | |

AUC_{0-24 h} = area under the plasma concentration vs. time curve from time zero to 24 hours post dose;
Cₘₐₓ = maximum plasma concentration;
F = female;
HED = human equivalent dose;
M = male;
MOS = margin of safety;
NOAEL = no-observed-adverse-effect-level;
PO = oral;
ROA = route of administration
^{c}Based upon study data interpretation and absence of any adverse toxicity.
^{d}Based on allometric conversion between species to achieve a HED. The allometric conversion factors used for mouse, minipig, and human are 3, 35, and 37, respectively.
^{e} Calculated by dividing the HED (g/day) study by the proposed human start dose of 0.5 g/day.
^{f} Calculated based on a 60 kg human.

In conclusion, available data from toxicity studies in normal mice and minipigs demonstrate a favorable safety profile at doses up to 1.5 g/kg/dose BID (3.0 g/kg/day) in mice and 1.0 g/kg/dose BID (2.0 g/kg/day) in minipigs, the highest doses tested and support the proposed clinical trial.

### Example 3: Clinical Testing for Safety and Pharmacokinetics

Toxicology studies have not revealed any untoward effects up the highest doses tested in 28-day toxicity studies in either species: 1.5 g/kg/dose BID (3.0 g/kg/day) in mice and 1.0 g/kg/dose BID (2.0 g/kg/day) in minipigs. Based on nonclinical data with chronic (6 months) administration in mice at doses (≥2 g/kg/day) exceeding those used in the toxicology studies, intestinal bloating and soft stool were observed, and attributed to an osmotic change resulting from ribitol treatment. This response subsided at 48 hours after termination of treatment. No other adverse findings have been identified thus far with ribitol. The food energy content of ribitol could not be found in the literature; its stereoisomer, xylitol, has a caloric content of 3 kcal/g, indicating that a 10 g daily dose would contribute less than 50 kcal to total daily caloric intake.

Ribitol is a pentose alcohol which is a stereoisomer of xylitol. Xylitol is generally regarded as safe (GRAS) by the US Food and Drug Administration (FDA) and used as a sweetening agent in products for human consumption. D-ribitol is an endogenous substance measurable in the blood and cerebrospinal fluid (CSF) of healthy human subjects at concentrations of approximately 0.5 micromolar (μM).

Ribitol is being developed for the treatment of patients with Limb-Girdle Muscular Dystrophy type 2i (LGMD2i) for which no approved therapy currently exists. This disease is characterized by a mutation in the enzyme involved in the glycosylation of α-dystroglycan and fukutin-related protein (FKRP), and results in hypoglycosylation. Glycosylation of α-dystroglycan plays a central role in maintaining muscle cell membrane integrity, and hypoglycosylation results in progressive muscle degeneration and loss of function. Over time, muscle is replaced by fibrotic tissue and fat. Ribitol targets the molecular defect at the source by supplying excess substrate to the mutant enzyme thus boosting glycosylation of muscle α-dystroglycan.

The study described below is a randomized, blinded, placebo-controlled, parallel group study of the administration of single ascending doses (SAD) and multiple ascending doses (MAD) of ribitol to healthy subjects. The SAD part of this study includes 1 food effect (FE) cohort. The SAD and MAD phases of the study are nested.

Up to 6 cohorts may be investigated in each part of this study for a maximum of 12 cohorts. Each cohort consists of 8 healthy subjects, randomized 6:2 to ribitol:placebo (“study drug” refers to ribitol or placebo). Within each cohort, 2 subjects must weigh between 40 and 50 kg; the remaining 6 subjects must weigh >50 kg. Up to 96 subjects may be enrolled in this study.

Objectives of the Phase 1 study include: evaluating the safety and tolerability of single and multiple doses of ribitol in healthy subjects; characterizing the single dose and steady state pharmacokinetics (PK) of ribitol in healthy subjects; and evaluating the effect of a standardized high calorie meal on the PK profile of ribitol.

### Single Ascending Dose (SAD) Cohorts

Up to a maximum of 6 cohorts will be enrolled. The starting dose will be 0.5 g. Tentative dose levels will be 1.5 g, 3 g, 6 g, and 12 g, however, actual dose increments (including possible decrements) for Cohorts 2 and above will be determined by the safety review committee (SRC) and based on a minimum of 24 h of post dose PK data and at least 72 h post dose safety data from the previous dose level. A sentinel dosing plan will be employed at each dose level where the first 2 healthy participants (1 ribitol, 1 placebo) of each cohort will be dosed as sentinels at least 24 h prior to dosing the remaining cohort's participants. Once the informed consent form (ICF) is signed and eligibility criteria are determined, subjects will be admitted to the clinical research unit (CRU) 1 day prior to dosing (Day −1). Continuous cardiac monitoring using telemetry will be performed starting at least 12 h prior to dosing through at least 24 h after dosing.

On Day 1, subjects will receive a single dose of oral study drug together with water orally (see Pharmacy Manual) after an overnight fast of at least 10 h prior to dosing and through at least 4 h after dosing. Water ad libitum is permitted except 1 h prior to through 1 h after dosing (other than that taken with the study drug). Subjects will remain in the CRU until Day 3 (This may be adjusted for Cohorts 2 and beyond to at least 3 half-lives up to a maximum of Day 7). Vital signs, electrocardiograms (ECG), evaluation of AEs and blood draws for PK and safety laboratory tests will be obtained serially (see Schedule of Assessments, below). Subjects will return for an end of study (EOS) visit on Day 7 (+3 day window allowed) or approximately 5 half-lives, whichever is longer, up to a maximum of Day 14. The PK sampling times and duration of sampling may be adjusted depending on the PK (half-life) results of prior cohorts up to a maximum of 8 additional blood draws.

Tentative dose levels for the cohorts will be:
SAD Cohort 1: 0.5 g
SAD Cohort 2: 1.5 g
SAD Cohort 3: 3 g
SAD Cohort 4: 6 g
SAD Cohort 5: 12 g
SAD Cohort 6: Dose to be determined but not to exceed 12 g.

### Single Ascending Dose—Food Effect (FE) Cohort

The SAD part of this study will include 1 FE cohort as selected by the SRC. The FE Cohort will consist of 2 treatment periods. The SRC will determine which cohort will participate in the FE portion of the study. That cohort will receive their dose twice in a cross-over fashion (once fasted [Treatment Period 1], once fed [Treatment Period 2]). The 2 periods will be separated by a washout period of at least 7 days or 5 half-lives, whichever is longer, up to 21 days. No sentinel dosing will be required for the second period.

### Treatment Period 1

During Treatment Period 1, subjects will be admitted to the CRU 1 day prior to dosing (Day −1). Subjects will be monitored, dosed, and assessed as described above. Water is allowed ad libitum except from 1 h prior to 1 h after dosing.

### Treatment Period 2

Subjects will return to the CRU for Treatment Period 2, one day prior to the second dose of study drug (Day −1). Subjects will be monitored, dosed, and assessed as described above.

On Day 1 of Treatment Period 2, subjects will consume a meal prior to receiving the second dose of study drug together with water orally.

The EOS visit for the FE Cohort will occur on Day 7 (+3 day window allowed) of Treatment Period 2 or approximately 5 half-lives, whichever is longer, but not more than 21 days.

### Duration of Study

The expected study duration for any individual subject will be up to 40 days (29 days for Screening Period, 4 days in-house, 3 days as outpatient, 1 day (+3) EOS visit). The maximum allowed duration based on allowable adjustment for actual PK data obtained in previous cohort(s) is 47 days. The expected study duration for the cohort participating in the FE 2-period crossover part will include an additional 8 days (4 days in-house, 3 days as outpatient, 1 day EOS visit).

### Multiple Ascending Dose (MAD) Cohorts

Up to a maximum of 6 cohorts will be enrolled. The starting dose for the first cohort and the dosing frequency (i.e., once daily, Q12h, Q8h) will be determined after the SRC has reviewed a minimum of 72 h of safety data and at least 24 h of PK data from 2 SAD cohorts. The starting dose and dosing frequency (i.e., once daily, every 8 h, or every 12 h) will be determined based on PK data obtained in the SAD cohorts. If dosing frequency is greater than once daily, dosing of MAD cohorts will begin once the FE data are available.

Subjects will be admitted to the CRU 1 day prior to dosing (Day −1). On Day 1, subjects will start a 6-day course of orally administered daily study drug together with water after an overnight fast of at least 10 h prior to through at least 4 h after dosing. If dosing occurs more than once per day based on the data from the SAD cohort(s), instructions for dosing and any relaxation of the fasting requirements will be provided in a separate document. Water ad libitum is permitted except 1 h prior to through 1 h after dosing. Subjects will remain in the CRU until Day 8. Vital signs, ECGs, evaluation of AEs and blood draws for PK and safety laboratory tests will be obtained serially (see Schedule of Assessments, below). Subjects will return for an EOS visit on Day 10 (+5 day window allowed).

Shorter dosing intervals (twice or three times daily administration) may be selected once the FE is known, in which case the fasting requirements may change. This will be outlined in a separate document. The dosing duration may be increased up to 12 days to allow for achievement of 3 half-lives plus 3 days (up to a maximum of 21 days [Day 20] in the CRU). Also, the PK sampling times and duration of sampling may be adjusted depending on the PK (half-life), up to a maximum of 8 additional blood draws. Similarly, the EOS visit may be adjusted to at least 5 half-lives, up to a maximum of Day 30.

### Duration of Study

The expected study duration for any individual subject will be up to 46 days (29 days Screening, 9 days in-house, 2 days as outpatient, 1 day (+5) EOS visit). The maximum allowed duration based on allowable adjustment for actual PK data obtained in the SAD phase and previous cohort(s) is 78 days.

### Enrollment and Study Procedures

Subjects will initially be assigned a screening identification number and will then be considered enrolled into the study once they have signed the ICF and have been determined to satisfy all inclusion and exclusion criteria. At the time of dosing, subjects will be assigned a subject randomization number. Details on the subject visits and clinical evaluations may be found in the Schedule of Assessments described below. Subjects will report to the study facility 1 day (Day −1) before the day of dosing (Day 1) and remain there for a total of 3 days (SAD) or 8 days (MAD), until the 48 h blood draw has been obtained after the last dose of study drug. When collection of PK samples, vital signs, and/or ECG assessments are scheduled to occur at the same time, priority should be given as follows: 1. PK collection (collected at nominal time) 2. Vital sign collection 3. ECG. Measurement of vital signs and ECG testing may be adjusted based on PK sample results.

### Subject Inclusion Criteria

Subjects must meet all the following criteria at Screening and upon admission (Day −1):
1. Healthy males and non-pregnant females, as determined by an essentially normal physical examination and normal laboratory screening tests, ages 18 to 65 years old and body mass index (BMI) 18 to 32 kg/m²
2. Willing and able to give informed consent and follow all study procedures and requirements
3. Willing to use adequate method of contraception from admission through 12 weeks after last administration. (See Section 5.3.3 for a description of adequate contraception.)
4. Willing to abstain from all alcoholic beverages and cannabinoids for 48 h prior to dosing through the EOS visit
5. Willing to abstain from caffeine and nicotine while in the CRU

### Subject Exclusion Criteria

Subjects must not meet any of the following criteria at Screening and upon admission (Day −1):

1. Evidence of clinically significant abnormalities or disease, including, the following: (a) Any history of any gastrointestinal condition, including surgeries, which may affect absorption after oral administration. (b) Abnormal blood pressure, defined as systolic >140 mmHg or <90 mmHg, and diastolic >90 mmHg or <50 mmHg. (c) Alanine aminotransferase (ALT), aspartate aminotransferase (AST), or bilirubin >1.5× the upper limit of normal (ULN). Subjects with bilirubin elevations due to Gilbert's Syndrome may be eligible for inclusion following discussion with the Medical Monitor. (d) Hemoglobin A1c≥6.5% and/or diagnosis of diabetes. (e) Positive test for human immunodeficiency virus (HIV) antibody. (f) Acute or chronic hepatitis B or C as evidenced by hepatitis B core antibody and/or hepatitis C antibody; evidence of resolve infection or status post (s/p) vaccination with the presence of antibodies. Documented absence of viral DNA on PCR is allowed. (g) History or electrocardiogram (ECG) evidence of myocardial ischemia or infarct, 2nd- or 3rd-degree atrioventricular block, bundle branch block with the exception of incomplete right bundle branch block in the absence of clinical heart disease, multiple or multifocal premature ventricular contractions, or QTcF>470 msec female or >450 msec male (average of 3 tracings). Additionally, subjects must be in sinus rhythm. (h) Any other laboratory, vital sign, ECG abnormality, or clinical history or finding that, in the investigator's opinion, is likely to unfavorably alter the risk-benefit of study participation, confound study results, or interfere with study conduct or compliance

2. Prior exposure to ribitol

3. Donation or loss of greater than 1 unit (450 mL) of blood within 1 month prior to dosing

4. Use of any prescription medication within 4 weeks or investigational medication within 12 weeks of dosing (exception: contraceptives are permitted)

5. Use of any non-prescription medication within 5 days prior to dosing (exception: acetaminophen up to 2 g per day prior to dosing is permitted).

6. If female, nursing, lactating, or pregnant

7. Known history of drug abuse and/or alcoholism within 2 years prior to screening, or positive screen for drugs of abuse or alcohol at Screening or Admission. Regular use of >5 cigarettes per day (or equivalent amount of nicotine containing product).

### Investigational Product, Dosage and Mode of Administration

Ribitol will be dosed orally with water after an overnight fasting period of at least 10 h and followed by fasting for at least 4 h. Water is permitted ad libitum except for 1 h before and 1 h after dosing. The FE cohort will receive study drug in Period 2 together with a standardized high calorie breakfast. If dosing in the MAD cohorts occurs more than once per day based on the data from the SAD cohort(s), instructions for dosing and any relaxation of the fasting requirements will be provided in a separate document.

Anticipated dose levels in SAD phase are 0.5 g, 1.5 g, 3 g, 6 g, 12 g, 15 g, 20 g, 25 g, and 30 g. The dose levels for the MAD part of the study will be determined based on the data from the SAD reviewed by the SRC.

Reference Therapy, Dose and Route of Administration: Matching placebo dosed orally.

### Analysis Populations

Three analysis populations are defined for this study.

The Safety Population is defined as all subjects who receive at least 1 dose of study drug and have at least 1 post-baseline safety assessment.

The PK Population is defined as all enrolled subjects for whom at least 1 PK parameter of interest can be calculated. In general, on a parameter-by-parameter basis, an individual subject's data may be excluded from analysis if insufficient data are available for that subject to calculate the specific parameter in question.

The Full Analysis Population is defined as all randomized subjects who complete the study without experiencing major protocol deviations or violations.

### Sample Size

The sample size is typical for first in human studies and allows a preliminary determination of tolerability safety and efficacy as well as a comprehensive determination of the single and multidose PK, including an estimate of the FE, in healthy subjects.

### Pharmacokinetic Analysis

The PK analyses will be performed using the PK population. Ribitol plasma concentrations will be summarized by dose and time point using descriptive statistics for the PK population. Mean and individual plasma ribitol concentrations over time will be presented in figures using linear and semilog scales. The PK parameters listed above under “Assessments” will be calculated using a non-compartmental analysis and summarized by dose. AUC and Cₘₐₓ will be tested across dose levels for dose proportionality; accumulation of AUC and Cₘₐₓ at steady state will be calculated. The amount of urinary drug excretion over 48 h (Ae₄₈ₕ) after single doses and over 24 h after the last dose (Ae₂₄ₕ) will be presented, and renal clearance (CLr) will be calculated.

### Schedule of Assessments for SAD Cohort and Treatment Period 1 of the FE Cohort

Hematology analysis (including hemoglobin, hematocrit, WBC, platelet count, and CBC differential) and urinalysis (including specific gravity, pH, glucose, protein, hemoglobin, leukocyte esterase, and nitrite) are measured four times over the course of the study: during screening, admission (Day −1), CRU Day 2, and the EOS visit. Vital signs and 12-lead ECG are measured every day of study participation as well as during screening. PK analysis (blood and urine collection) samples are collected: on Day 1: within 30 min predose, 0.25, 0.5, 1, 1.5, 2, 3, 4, 6, 8, 12, 18 h, 24 h, 36 h, and 48 h post dose. A urine sample will be obtained for PK within 2 h prior to dosing, followed by a 48 h urine collection for PK in 8 h aliquots.

### Schedule of Assessments for Treatment Period 2 of the FE Cohort

Hematology analysis (including hemoglobin, hematocrit, WBC, platelet count, and CBC differential) and urinalysis (including specific gravity, pH, glucose, protein, hemoglobin, leukocyte esterase, and nitrite) are measured three times over the course of the study: during screening, CRU Day 2, and the EOS visit. Vital signs and 12-lead ECG are measured every day of study participation as well as during screening. PK analysis (blood collection) samples are collected: Day 1: within 1 hour predose, and 0.25, 0.5, 1, 1.5, 2, 3, 4, 6, 8, 12, 18 h, 24 h, 36 h and 48 h post dose.

### Schedule of Assessments for MAD Cohort

Hematology analysis (including hemoglobin, hematocrit, WBC, platelet count, and CBC differential) and urinalysis (including specific gravity, pH, glucose, protein, hemoglobin, leukocyte esterase, and nitrite) are measured six times over the course of the study: during screening, admission (Day −1), CRU Day 2, CRU Day 5, CRU Day 8, and the EOS visit. Vital signs and 12-lead ECG are measured every day of study participation as well as during screening. PK analysis (blood and urine collection) samples are collected: on Day 1: within 1 h predose, and at 0.25, 0.5, 1, 1.5, 2, 3, 4, 6, 8, 12, 18 h post first-dose. On days 2-5: within 30 minutes prior to the first dose and at 0.5 h post first dose. On day 6: within 30 minutes prior to the first dose, and at 0.25, 0.5, 1, 1.5, 2, 3, 4, 6, 8, 12, 18, 24, 36, and 48 h post first dose. A urine sample will be obtained for PK analysis within 2 h of dosing; a 24 h collection in 8 h aliquots beginning with the last dose of study drug.

### Safety Analysis

The safety analyses will be assessed based on the evaluation of treatment emergent adverse events (TEAE) for the entire study. All AEs will be coded using the Medical Dictionary for Regulatory Activities (MedDRA) version 19.1 or later. Safety data, including AEs, clinical laboratory test results, including main intervals on 12-lead ECGs, and vital sign measurements, will be summarized by dose and placebo. Physical examination findings and concomitant medications will be listed.

### Meals and Activities

While subjects are domiciled in the study unit, they will receive standardized meals on a fixed schedule with meal content to be specified according to the study unit protocol. Meals will be identical for all subjects and will be provided at the same time, relative to dosing, each day during the treatment period and per clinic protocol. In particular, the timing and menu items for the meals on Days −1, Day 1, as well as the last dosing day in the MAD phase should be identical. An overnight fast of at least 10 h must be observed prior to study drug administration and for at least 4 h thereafter. If dosing occurs more than once per day based on results from the SAD phase, instructions for dosing and any relaxation of the fasting requirements will be provided in a separate document.

On Day 1 of Treatment Period 2, subjects will consume a meal prior to receiving the second dose of study drug together with water orally. The meal will consist of a standardized high calorie breakfast as described in the FDA Guidance for Industry document (Food-Effect Bioavailability and Fed Bioequivalence Studies; December 2002). The meal is to be ingested entirely within 30 minutes and study drug will be administered 30 minutes after the start of the meal. Water is allowed ad libitum except from 1 h prior to 1 h after dosing.

### Treatments Administered

Subjects will be randomized to receive ribitol or placebo in a blinded fashion. Ribitol and placebo dose will be identical in all aspects including volume, and color. Immediately prior to dosing, subjects may be given a taste masking agent before dosing to mask any flavor differences between ribitol and placebo.

### Pharmacokinetic Assessments

Plasma and urine samples will be collected for the determination of concentrations ribitol, CDP-ribitol, and possibly other metabolites. Sample collection times are provided in the Schedule of Assessments described above. Details on the handling of PK specimens are found in a separate laboratory manual.

The following PK parameters will be calculated for all subjects:
Terminal half-life (t_{1/2}), determined by linear regression of the log concentration on the distribution and terminal portion of the plasma concentration-time curve. Terminal half-life is calculated as ln(2)/(−β), where β is the slope of the terminal portion of the log concentration-time curve
Time to maximum observed plasma concentration (Tₘₐₓ)
Maximum observed plasma concentration (Cₘₐₓ)
SAD Cohort PK Assessment:
AUC₀₋ₗₐₛₜ, AUC₀₋₂₄, AUC₀₋₄₈, and AUC_{0-∞} (computed as: AUC_{0-∞}=AUC₀₋ₗₐₛₜ+Cₚₗₐₛₜ/(−β) where Cₚₗₐₛₜ is the last measurable plasma concentration
Percent AUC_{0-∞} extrapolated
Amount excreted in urine over 48 h (Ae₄₈)
Renal clearance (CLr)=Ae₄₈/AUC₀₋₄₈
Dose-normalized AUC_{0-∞} [AUC_{0-∞}/D] and Cₘₐₓ (Cₘₐₓ/D)
Apparent clearance (CL/F)
Apparent volume of distribution (V_{z}/F)
MAD Cohort PK Assessment:
Area under the plasma concentration-time curve for the dosing interval (AUC_{0-τ}) after the first and last dose
Accumulation ratio (RAUC)=Last Day AUC₀₋₂₄/Day 1 AUC₀₋₂₄
Amount excreted in urine over 24 h after the last dose (Ae₂₄)
CLr=Ae₂₄/AUC₀₋₂₄
CL/F
V_{z}/F

### Safety Review Committee

A Safety Review Committee (SRC) consisting of the Principal Investigator, the ML Bio Medical Monitor and an independent physician will review PK and safety data obtained up to 24 h following a single dose in the SAD phase and following the final dose in the MAD phase for each cohort as long as the safety data during the period of confinement supports subsequent dose escalation. Data from a minimum of six out of the planned eight subjects in a given cohort must be available for the SRC to convene and make decisions regarding dose ascension.

### Packaging

The bulk drug substance is packaged in low density polyethylene (LDPE) bags with a desiccant and closed with ties. The drug substance inside the LDPE bags is placed inside a foil bag and sealed; the sealed foil bag is then placed inside a high-density polyethylene (HDPE) drum. The bulk drug substance is received by the compounding pharmacy at the clinical site and formulated for administration to the study subjects.

### Minimization of Bias

Immediately prior to dosing, subjects will be given a breath strip to mask any flavor differences between ribitol and placebo. The investigator, CRU staff, and subject are blinded as to treatment assignment, and individual treatment assignment is randomized. The study pharmacist will remain unblinded.

### Example 4: 1 Year Oral Dosing of Ribitol in L267I FKRP Mutant Mice at Doses of 0.5 g/Kg, 2 g/kg, 5 g/kg, 10 g/kg and 5 g/kg BID

The effect of ribitol on an additional model of LGMD2i was evaluated in mice with the L276I FKRP mutation. L276I mutant mice were treated with ribitol at single daily doses of 0.5 g/kg, 2 g/kg, 5 g/kg, and 5 g/kg BID by oral gavage from the age of 8 weeks when the mild dystrophic phenotype is detectable. The duration of treatment was one year. Ribitol was dissolved in saline and administered by oral gavage. The same methods described above were applied to evaluate expression of matriglycan and muscle pathology.

FIG. 14 shows that the creatine kinase (a general marker of muscle damage) is reduced with increasing doses of ribitol. FIG. 15 shows that the expression of the matriglycan on αDG increases in a dose dependent manner with immunohistochemistry. The data suggests that the decreases in creatine kinase result from the increased matriglycan expression on αDG which stabilizes the muscle fiber, preventing the release of creatine kinase

In addition to biomarker changes, clinical changes were observed in the mice with statistically significant improvement at 2 g/kg/day, as described in Example 2 above. For the P448L mice model, at 6 months treadmill distance increased 50% and running time increased 50-75% (FIGS. 4A and 4B). In the L276I mouse a 14% increase in treadmill running distance was also observed at 2 g/kg/day (FIGS. 7B and 7C).

Accordingly, a daily dose of at least about 0.5 g/kg/day may be effective in improving biomarkers including CK, and daily doses of at least about 2 g/kg/day may yield a clinical benefit.

### Example 5: Healthy Volunteer Studies—Establishing Presumption for Efficacious Dose Based on TK/AUC Exposure

The exposure following 0.5-2 g/kg/day was estimated from using data from other studies. The average pharmacokinetic parameters of ribitol following 3 g/kg/day are shown in Table 15.

TABLE 15
Steady-state AUC(0-24) of ribitol following 1.5
g/kg/dose of ribitol Twice Daily (e.g., 3 g/kg/day)
in 1, 3, or 6 Months Toxicology Studies

| | Steady-state AUC(0-24) (μg*h/mL) at 3 g/kg/day | | | |
|---|---|---|---|---|
| | 1-Month | 3-Month | 6-Month | Mean |
| Male | 1340 | 974 | 1540 | 1285 |
| Female | 839 | 749 | 1100 | 896 |
| Mean (M + F) | 1090 | 862 | 1320 | 1090 |

The average AUC(0-24) of ribitol following 3 g/kg/day from the three studies was 1090 μg*h/mL. Since the exposure of ribitol increases proportionally to dose, the exposure at 2 g/kg/day was calculated to be 727 μg*h/mL [1090*2/3=727 μg*h/mL]. The AUC(0-24) at 0.5 mg/kg/day was calculated to be 182 μg*h/mL [1090*0.5/3=182 μg/h/mL].

### Example 6: Healthy Volunteer Studies

In the Phase 1 study, conducted essentially as described above, in Example 3. Subject in the single dose arm received 0.5, 1.5, 3, 6, 9, 12, or 15 grams of ribitol (data not shown). At these dose levels, ribitol was well tolerated and no dose limiting toxicity was observed. Subjects in the multiple dose arm received 1.5 g once daily, 3 g once daily, 3 g twice daily (q 12 h), 6 g twice daily (q 12 h), and 9 g twice daily (q 12 h). Exposure values are provided in Table 16. Subjects in the 3 g BID arm achieved steady-state AUC(0-24) values about the target threshold set by animal studies (182 μg*h/mL).

TABLE 16
Steady-state pharmacokinetic parameters of ribitol
following Twice Daily Dosing (q 12 h) for 6 Days

| Dose (mg) | Tmax (h) | Cmax (μg/mL) | AUC(0-24) (μg*h/mL) |
|---|---|---|---|
| 1.5 g once daily | 0.5040 | 24.97 | 59.78 |
| 3 g once daily | 0.5030 | 35.55 | 94.97 |
| 3 g q 12 h (total 6 | 0.5010 | 37.97 | 218.8 |
| g/day) | | | |
| 6 g q 12 h (total 12 | 0.7505 | 107.2 | 500.0 |
| g/day) | | | |
| 9 g q 12 h (total 18 | 0.5010 | 143.1 | 673.2 |
| g/day) | | | |

Given efficacy in the animal model studies at AUC₀₋₂₄₌₁₈₂ μg*h/mL, the Phase 1 study targeted an exposure at at least this level. Based on the pharmacokinetic data from the healthy volunteer study, this exposure can be achieved at a human dose of at least 3 g q 12 hours (BID). However, high doses were well tolerated. The dose of 3 g BID (6 g/day) in human had exposure similar to the 0.5 g/kg/day in the mouse; and 9 g BID (18 g/day) in human had exposure close to the 2 g/kg/day in the mouse.

### Example 7: Phase 2 Patient Studies—Identifying/Confirming Efficacious Dose Based on Clinical Trial Data

Ribitol is being tested in an ongoing Phase 2 study with 14 patients receiving doses of either 6 g once daily (QD), 6 g twice daily (BID), or 12 g BID. Data is available following 3 months of therapeutic exposure in 8 subjects (6 g QD, n=4, 6 g BID, n=4). Treatment at 6 g QD or 6 g BID causes increases in glycosylation of αDG in 4 of 4 subjects in the lower dose cohort and 3 of 4 subjects in the higher dose cohort with an average increase of 13% for both cohorts (FIG. 16). Furthermore, decreased muscle damage—likely related to increase in αDG—was observed in all eight subjects with an average decrease for the 6 g QD cohort of 90% and 77% for the 6 g BID cohort at day 90 (FIG. 17). The 12 g BID cohort showed an average decrease in creatine kinase of 33% at day 8. All 12 subjects have shown decreases in creatine kinase from baseline. Younger, ambulatory patients started with higher baseline creatine kinase values and had the largest magnitude drops. Decreases were observable starting at day 8 with continued declines through day 90.

The combination of αDG and creatine kinase is compelling biomarker evidence linking increased glycosylation to decreased muscle breakdown in prior studies (Cataldi et al., Nature Communications 9:3448 (2018)).

Efficacy studies are ongoing. Improvements in 10-meter walk test (MWT) to test ambulation, NSAD for gross motor function, PUL2.0 for upper limb function, and spirometry for respiratory function have been observed (Table 17). Published Natural History data suggests there is a slow annual decline in motor function in adult with FKRP mutations that can be detected with standard motor outcomes while changes in the pediatric population are most variable and affected by genotype (Gedlinske, et al, 2020). Increases and stability at 3-months has demonstrated, with continued follow-up planned.

Decreases in patient-reported muscle fatigue over the 90-day treatment period has been observed in 7 of 8 patients. These 7/8 patients have reported decreases as recorded in a daily diary and noted on a scale of 1 to 10 where 1 indicates no fatigue and a 10 is the maximum level of fatigue. The average drop in fatigue across for cohort 1 over 90-days was 1.3 points and 1.5 points for cohort 2 (Table 17).

TABLE 17
Clinical outcomes data for cohort 1&2 at 90-days showing
small improvements in 10MWT of ~5% from baseline as
well as stability across the NSAD and PUL2.0 assessments.

| Parameter | | | Cohort 1 | | Cohort 2 | | Overall | |
|---|---|---|---|---|---|---|---|---|
| # | | | 4 | | 4 | | 8 | |
| Peds/adults | | | 2/2 | | 2/2 | | 2/2 | |
| Homs/Hets | | | 3/1 | | 2/2 | | 5/3 | |
| Mean | Ambulation | Baseline (SD) | 2.06 | (1.50) | 1.48 | (1.29) | 1.77 | (1.33) |
| 10MWT | | Day 90 (SD) | 2.82 | (1.01) | 1.56 | (1.46) | 2.10 | (1.36) |
| (m/sec) | | Delta (SD) | 0.10 | (0.11) | 0.08 | (0.17) | 0.09 | (0.13) |
| Mean | Gross Motor | Baseline (SD) | 33.8 | (19.60) | 28.5 | (18.19) | 31.1 | (17.73) |
| NSAD | Function | Day 90 (SD) | 33.5 | (21.21) | 29.0 | (17.05) | 31.3 | (17.97) |
| | | Delta (SD) | −0.3 | (1.89) | 0.5 | (3.70) | 0.1 | (2.75) |
| Mean | Upper Limb | Baseline (SD) | 37.3 | (8.85) | 35.8 | (7.50) | 36.5 | (7.63) |
| PUL2.0 | Function | Day 90 (SD) | 37.3 | (8.85) | 36.3 | (6.29) | 36.8 | (7.13) |
| | | Delta (SD) | 0.0 | (0.82) | 0.5 | (1.29) | 0.3 | (1.04) |
| Mean PP | Respiratory | Baseline (SD) | | | 54.3 | (14.08) | 54.3 | (14.08) |
| Supine | Function | Day 90 (SD) | 79.5 | (23.90) | 50.0 | (15.10) | 64.8 | (24.31) |
| FVC | | Delta (SD) | | | −4.3 | (5.32)1 | −4.3 | (5.32)1 |

### Example 8: Clinical Testing for Safety and Pharmacokinetics

Clinical studies were performed as described in Example 3, with the exception that two additional doses of ribitol (9 g and 15 g) were administered in the SAD arm, i.e., the SAD doses were 0.5 g, 1.5 g, 3 g, 6 g, 9 g, 12 g, and 15 g ribitol. The results of this study confirmed dose linearity with exposure.

ABBREVIATIONS

| Abbreviation | Definition |
|---|---|
| μg | microgram |
| α-DG | alpha-dystroglycan |
| β-DG | beta-dystroglycan |
| ADME | absorption, distribution, metabolism and elimination |
| AE | Adverse event |
| Ae | Amount excreted (urine) |
| AF6868 | Sheep Anti-Human Dystroglycan Antigen Polyclonal |
| | Antibody |
| ALT | Amino alanine transferase |
| AST | Amino aspartate transferase |
| AUC | Area under the concentration over time curve |
| AUC₀₋₂₄ | area under the curve from time zero to 24 hours post first |
| | dose |
| AUC₀₋ₗₐₛₜ | area under the curve from time zero to the last measurable |
| | concentration |
| B3GALNT2 | beta-1,3-N-Acetylgalactosaminyltransferase 2 |
| B4GAT1 | beta-1,4-Glucuronyltransferase 1 |
| BID | Twice daily |
| BLQ | below limit of quantitation |
| BP | Blood pressure |
| BUN | Blood urea nitrogen |
| C57 | C57BL/6 is a common inbred strain of laboratory mouse |
| | used as “genetic background” for genetically modified |
| | mice for use as models of human disease |
| CBC | Complete blood count |
| CDP-ribitol | Cytidine 5-diphosphotase ribitol |
| CFR | Code of Federal Regulations |
| CL | Clearance |
| CL/F | Apparent clearance |
| CLr | Renal clearance |
| CLₛ | systemic clearance |
| Cₘₐₓ | maximum observed concentration |
| CNF | centrally nucleated fibers |
| CRF | Case report forms |
| CRU | Clinical research unit |
| CV | Coefficient of variation |
| CNS | central nervous system |
| d(s) | Day(s) |
| DAG1 | dystroglycan 1 |
| Dia/Diaph | Diaphram |
| DGC | dystrophin-associated glycoprotein complex |
| DRF | dose range finding |
| ECG | electrocardiogram |
| EOS | End of study |
| FDA | Food and Drug Administration |
| FE | Food effect |
| FKRP | Fukutin-related protein |
| FKTN | fukutin |
| FOB | functional observational battery |
| FSH | Follicle stimulating hormone |
| g | gram(s) |
| GCP | Good Clinical Practice |
| GLP | Good Laboratory Practice |
| GMR | Geometric mean ratio |
| GRAS | Generally recognized as safe |
| h | Hour(s) |
| HDPE | High-density polyethylene |
| HED | Human equivalent dose |
| H&E | hematoxylin and eosin |
| hERG | human ether-a-go-go-related gene |
| HPBL | human peripheral blood lymphocytes |
| HR | Heart rate |
| ICF | Informed consent form |
| ICH | International Council for Harmonization of Technical |
| | Requirements for Pharmaceuticals for Human Use |
| IIH6C4 | monoclonal anti-a-dystroglycan antibody |
| I_{Kr} | rapidly activating delayed rectifier potassium |
| IND | Investigational New Drug Application |
| IRB | Institutional Review Board |
| ISPD | isoprenoid synthase domain containing protein |
| IV | intravenous |
| IXRS | Interactive Voice/Web Response System |
| Kcal | kilocalorie |
| kg | kilogram(s) |
| K_{M} | Michaelis constant |
| L | liter |
| LARGE | like-glycosyltransferase |
| LC-MS/MS | liquid chromatography, tandem mass spectrometry |
| LDPE | Low-density polyethylene |
| LGMD | limb girdle muscular dystrophy |
| LGMD2i | limb girdle muscular dystrophy type 2i |
| MAD | multiple ascending dose |
| mg | milligram(s) |
| mM | millimolar |
| mRNA | messenger ribonucleic acid |
| MS | mass spectrometry |
| MSE | Mean square error |
| MTD | maximum tolerated dose |
| NOEL | no observed effect level |
| NOAEL | no observed adverse effect level |
| PAD | Pharmacologically active dose |
| PK | Pharmacokinetic(s) |
| P.O. | dosage administration “per os”, by mouth |
| POMT1/2 | protein O-mannosyltransferase |
| POMGnT2 | protein O-linked mannose |
| | N-acetylglucosaminyltransferase 2 |
| QD | once daily |
| RAUC | Accumulation ratio |
| rib | ribitol |
| Ribitol 5P | ribitol-5-phosphate |
| SAD | single ascending dose |
| SAE | Serious adverse event |
| SD | Standard deviation |
| SEM | Standard error of the mean |
| SRC | Safety Review Committee |
| t_{1/2} | Terminal half-life |
| TA | Tibialis anterior |
| TEAE | Treatment emergent adverse event |
| TID | Three times daily |
| TK | Toxicokinetics |
| Tₘₐₓ | Time at which Cmax is observed |
| TMEM | transmembrane protein 5 |
| TSH | Thyroid stimulating hormone |
| ULN | Upper limit of normal |
| USP | United States Pharmacopeia |
| Vd | volume of distribution |
| Vₛₛ | Volume of distribution at steady state |
| Vₛₛ/F | Apparent volume of distribution at steady-state |
| WBC | White blood cell(s) |

What is claimed is:

1. A method of treating a disease or disorder in a subject in need thereof, comprising administering a dose comprising an effective amount of ribitol, optionally an immediate-release dose and/or not an controlled-release dose.

2. The method of claim 1, wherein the dose is administered at most four times daily.

3. The method of claim 2, wherein the dose is administered at most twice daily.

4. The method of claim 1, wherein the dose is administered three times daily.

5. The method of claim 3, wherein the dose is administered twice daily.

6. The method of claim 3, wherein the dose is administered once daily.

7. The method of any of claims 1 to 5, wherein the method comprises administering at least 0.5 grams per day (g/day), at least 1 g/day, at least 2 g/day, at least 3 g/day, at least 4 g/day, at least 5 g/day, at least 7.5 g/day, at least 10 g/day, at least 12.5 g/day, at least 15 g/day, at least 20 g/day, at least 25 g/day, at least 30 g/day, at least 35 g/day, at least 40 g/day, at least 45 g/day, at least 50 g/day, at least 55 g/day, or at least 60 g/day.

8. The method of any of claims 1 to 6, wherein the method comprises administering at most 0.5 g/day, at most 1 g/day, at most 2 g/day, at most 3 g/day, at most 4 g/day, at most 5 g/day, at most 7.5 g/day, at most 10 g/day, at most 12.5 g/day, or at most 15 g/day, at most 20 g/day, at most 25 g/day, at most 30 g/day, at most 35 g/day, at most 40 g/day, at most 45 g/day, at most 50 g/day, at most 55 g/day, or at most 60 g/day.

9. The method of any of claims 1 to 5, wherein the method comprises administering 0.5 g/day, 1 g/day, 1.5 g/day, 2 g/day, 3 g/day, 4 g/day, 5 g/day, 6 g/day, 7.5 g/day, 10 g/day, 12 g/day, 12.5 g/day, 15 g/day, 20 g/day, 25 g/day, 30 g/day, 35 g/day, 40 g/day, 45 g/day, 50 g/day, 55 g/day, or 60 g/day.

10. The method of claim 8, wherein the method comprises administering 0.5 g/day.

11. The method of claim 8, wherein the method comprises administering 1.5 g/day.

12. The method of claim 8, wherein the method comprises administering 3 g/day.

13. The method of claim 8, wherein the method comprises administering 6 g/day.

14. The method of claim 8, wherein the method comprises administering 10 g/day.

15. The method of claim 8, wherein the method comprises administering 12 g/day.

16. The method of claim 8, wherein the method comprises administering 15 g/day.

17. The method of claim 8, wherein the method comprises administering 20 g/day.

18. The method of claim 8, wherein the method comprises administering 25 g/day.

19. The method of claim 8, wherein the method comprises administering 30 g/day.

20. The method of claim 8, wherein the method comprises administering 35 g/day.

21. The method of claim 8, wherein the method comprises administering 40 g/day.

22. The method of claim 8, wherein the method comprises administering 45 g/day.

23. The method of claim 8, wherein the method comprises administering 50 g/day.

24. The method of claim 8, wherein the method comprises administering 55 g/day.

25. The method of claim 8, wherein the method comprises administering 60 g/day.

26. The method of any one of claims 1 to 25, comprising administering the dose of ribitol for at least one week, two weeks, or four weeks.

27. The method of any one of claims 1 to 25, comprising administering the dose of ribitol for at least one month, two months, or four months.

28. The method of any one of claims 1 to 25, comprising administering the dose of ribitol chronically.

29. The method of any one of claims 1 to 28, wherein the disease or disorder is associated with a defect in Fukutin-related protein (FKRP).

30. The method of any one of claims 1 to 29, wherein a mammal has a mutation in the gene encoding Fukutin-related protein (FKRP) that causes a partial or complete loss-of-function in FKRP.

31. The method of any one of claims 1 to 30, wherein the disease or disorder is a muscular dystrophy.

32. The method of claim 31, wherein the muscular dystrophy is FKRP-related alpha-dystroglycanopathy.

33. The method of claim 32, wherein the disease or disorder is Limb-Girdle Muscular Dystrophy type 2i (LGMD2i).

34. The method of claim 31, wherein the muscular dystrophy is a fukutin (FKTN)-related alpha-dystroglycanopathy.

35. The method of claim 34, wherein the FKTN-related alpha-dystroglycanopathy is Fukuyama Syndrome.

36. The method of any one of claims 1 to 35, wherein the maximum observed concentration (Cₘₐₓ) of ribitol is between 50 and 2500 μg/mL.

37. The method of any one of claims 1 to 36, wherein the area under the concentration-time curve (AUC₀₋₂₄) for ribitol is between 100 (μg·h)/mL and 8000 (μg·h)/mL or between 350 (μg·h)/mL and 8000 (μg·h)/mL.

38. The method of any one of claims 1 to 36, wherein the AUC₀₋₂₄ for ribitol is at least about 100 (μg·h)/mL or about 100 (μg·h)/mL to about 700 (μg·h)/mL.

39. The method of any one of claims 1 to 36, wherein the AUC₀₋₂₄ for ribitol is at least about 182 (μg·h)/mL or about 182 (μg·h)/mL to about 700 (μg·h)/mL.

40. The method of any one of claims 1 to 36, wherein the AUC₀₋₂₄ for ribitol is at least about 200 (μg·h)/mL or about 200 (μg·h)/mL to about 700 (μg·h)/mL.

41. The method of any one of claims 1 to 36, wherein the AUC₀₋₂₄ for ribitol is at least about 700 (μg·h)/mL or about 500 (μg·h)/mL to about 700 (μg·h)/mL.

42. The method of any one of claims 1 to 41, wherein the subject is a mammal.

43. The method of any one of claims 1 to 41, wherein the subject is a human.

44. The method of any one of claims 1 to 43, wherein the subject is a human child.

45. The method of any one of claims 1 to 44, wherein the method restores and/or enhances functional glycosylation of α-DG.

46. The method of any one of claims 1 to 44, wherein the method treats the disease or disorder.

47. A method of treating a disease or disorder in a subject in need thereof, comprising administering ribitol at dose effective to achieve steady-state AUC(0-24) level.

48. The method of claim 47, wherein the dose is administered at most four times daily.

49. The method of claim 48, wherein the dose is administered at most twice daily.

50. The method of claim 47, wherein the dose is administered three times daily.

51. The method of claim 49, wherein the dose is administered twice daily.

52. The method of claim 49, wherein the dose is administered once daily.

53. The method of any of claims 47 to 52, wherein the method comprises administering at least 0.5 grams per day (g/day), at least 1 g/day, at least 2 g/day, at least 3 g/day, at least 4 g/day, at least 5 g/day, at least 7.5 g/day, at least 10 g/day, at least 12.5 g/day, at least 15 g/day, at least 20 g/day, at least 25 g/day, at least 30 g/day, at least 35 g/day, at least 40 g/day, at least 45 g/day, at least 50 g/day, at least 55 g/day, or at least 60 g/day.

54. The method of any of claims 47 to 52, wherein the method comprises administering at most 0.5 g/day, at most 1 g/day, at most 2 g/day, at most 3 g/day, at most 4 g/day, at most 5 g/day, at most 7.5 g/day, at most 10 g/day, at most 12.5 g/day, or at most 15 g/day, at most 20 g/day, at most 25 g/day, at most 30 g/day, at most 35 g/day, at most 40 g/day, at most 45 g/day, at most 50 g/day, at most 55 g/day, or at most 60 g/day.

55. The method of any of claims 47 to 52, wherein the method comprises administering 0.5 g/day, 1 g/day, 1.5 g/day, 2 g/day, 3 g/day, 4 g/day, 5 g/day, 6 g/day, 7.5 g/day, 10 g/day, 12 g/day, 12.5 g/day, 15 g/day, 20 g/day, 25 g/day, 30 g/day, 35 g/day, 40 g/day, 45 g/day, 50 g/day, 55 g/day, or 60 g/day.

56. The method of claim 54, wherein the method comprises administering 0.5 g/day.

57. The method of claim 54, wherein the method comprises administering 1.5 g/day.

58. The method of claim 54, wherein the method comprises administering 3 g/day.

59. The method of claim 54, wherein the method comprises administering 6 g/day.

60. The method of claim 54, wherein the method comprises administering 10 g/day.

61. The method of claim 54, wherein the method comprises administering 12 g/day.

62. The method of claim 54, wherein the method comprises administering 15 g/day.

63. The method of claim 54, wherein the method comprises administering 20 g/day.

64. The method of claim 54, wherein the method comprises administering 25 g/day.

65. The method of claim 54, wherein the method comprises administering 30 g/day.

66. The method of claim 54, wherein the method comprises administering 35 g/day.

67. The method of claim 54, wherein the method comprises administering 40 g/day.

68. The method of claim 54, wherein the method comprises administering 45 g/day.

69. The method of claim 54, wherein the method comprises administering 50 g/day.

70. The method of claim 54, wherein the method comprises administering 55 g/day.

71. The method of claim 54, wherein the method comprises administering 60 g/day.

72. The method of any one of claims 1 to 71, comprising administering the dose of ribitol for at least one week, two weeks, or four weeks.

73. The method of any one of claims 1 to 71, comprising administering the dose of ribitol for at least one month, two months, or four months.

74. The method of any one of claims 1 to 71, comprising administering the dose of ribitol chronically.

75. The method of any one of claims 1 to 71, wherein the disease or disorder is associated with a defect in Fukutin-related protein (FKRP).

76. The method of any one of claims 47 to 75, wherein a mammal has a mutation in the gene encoding Fukutin-related protein (FKRP) that causes a partial or complete loss-of-function in FKRP.

77. The method of any one of claims 47 to 76, wherein the disease or disorder is a muscular dystrophy.

78. The method of claim 77, wherein the muscular dystrophy is FKRP-related alpha-dystroglycanopathy.

79. The method of claim 78, wherein the disease or disorder is Limb-Girdle Muscular Dystrophy type 2i (LGMD2i).

80. The method of claim 77, wherein the muscular dystrophy is a fukutin (FKTN)-related alpha-dystroglycanopathy.

81. The method of claim 80, wherein the FKTN-related alpha-dystroglycanopathy is Fukuyama Syndrome.

82. The method of any one of claims 47 to 81, wherein the maximum observed concentration (Cₘₐₓ) of ribitol is between 50 and 2500 μg/mL.

83. The method of any one of claims 47 to 82, wherein the area under the serum concentration-time curve (AUC₀₋₂₄) for ribitol is between 350 (μg·h)/mL and 8000 (μg·h)/mL.

84. The method of any one of claims 47 to 82, wherein the AUC₀₋₂₄ for ribitol is at least about 100 (μg·h)/mL.

85. The method of any one of claims 47 to 82, wherein the AUC₀₋₂₄ for ribitol is at least about 182 (μg·h)/mL.

86. The method of any one of claims 47 to 82, wherein the AUC₀₋₂₄ for ribitol is at least about 200 (μg·h)/mL.

87. The method of any one of claims 47 to 82, wherein the AUC₀₋₂₄ for ribitol is at least about 700 (μg·h)/mL.

88. The method of any one of claims 47 to 87, wherein the subject is a mammal.

89. The method of any one of claims 47 to 87, wherein the subject is a human.

90. The method of any one of claims 1 to 89, wherein the subject is a human child.

91. The method of any one of claims 1 to 90, wherein the method restores and/or enhances functional glycosylation of α-DG.

92. The method of any one of claims 1 to 89, wherein the method treats the disease or disorder.

93. A pharmaceutical composition, comprising ribitol and a pharmaceutically acceptable carrier or excipient.

94. The pharmaceutical compositions of claim 93, wherein the pharmaceutical composition is a solid, optionally a tablet or capsule.

95. The pharmaceutical compositions of claim 93, wherein the pharmaceutical composition is a solution.

96. The pharmaceutical composition of claim 95, wherein the carrier is water.

97. The pharmaceutical composition of claim 96, wherein the carrier is substantially pure water.

98. The pharmaceutical composition of claim 97, wherein the carrier is saline.

99. The pharmaceutical composition of any one of claims 93 to 98, wherein the pharmaceutical composition comprises ribitol at between 0.2 g/mL and 10 g/mL.

100. A kit comprising the pharmaceutical composition of any one of claims 93 to 99 and instructions for use in treating a disease or disorder.

101. A unit dose, comprising between 0.5 g and 60 g of ribitol.

102. The unit dose of claim 101, wherein the unit dose comprising 0.5 g of ribitol.

103. The unit dose of claim 101, wherein the unit dose comprising 1.5 g of ribitol.

104. The unit dose of claim 101, wherein the unit dose comprising 3 g of ribitol.

105. The unit dose of claim 101, wherein the unit dose comprising 6 g of ribitol.

106. The unit dose of claim 101, wherein the unit dose comprising 9 g of ribitol.

107. The unit dose of claim 101, wherein the unit dose comprising 12 g of ribitol.

108. The unit dose of claim 101, wherein the unit dose comprising 15 g of ribitol.

109. The unit dose of any one of claims 101-108, wherein the ribitol is dissolved in water.

110. A unit dose, comprising an amount of ribitol that is effective to achieve a steady-state AUC(0-24) level for ribitol of between 100 (μg·h)/mL and 8000 (μg·h)/mL.

111. The unit dose of claim 110, wherein the AUC₀₋₂₄ for ribitol is at least about 100 (μg·h)/mL or about 100 (μg·h)/mL to about 700 (μg·h)/mL.

112. The unit dose of claim 110, wherein the AUC₀₋₂₄ for ribitol is at least about 182 (μg·h)/mL or about 182 (μg·h)/mL to about 700 (μg·h)/mL.

113. The unit dose of claim 110, wherein the AUC₀₋₂₄ for ribitol is at least about 200 (μg·h)/mL or about 200 (μg·h)/mL to about 700 (μg·h)/mL.

114. The unit dose of claim 110, wherein the AUC₀₋₂₄ for ribitol is at least about 700 (μg·h)/mL or about 500 (μg·h)/mL to about 700 (μg·h)/mL.

115. The unit dose of any one of claims 101-114, wherein the unit dose is formulated as a solid, optionally a tablet or capsule.

116. The unit dose of any one of claims 101-114, wherein the unit dose is formulated as a liquid, optionally wherein the ribitol is dissolved in water.

117. The unit dose of any one of claims 101-116, wherein the unit dose is formulated for oral administration.

118. The pharmaceutical composition of any one of claims 93-99 or unit dose of any one of claims 101-106 for use in a method of treatment according to any one of claims 1-92.
